(19) United States
(12) Patent Application Publication
(10) Pub. No.: US 2016/0058878 A1
(43) Pub. Date: Mar. 3, 2016

(54) Process for preparing a composition of pegylated proteins

(71) Applicants: Simona JEVSEVAR, Ljubljana (SI); Menci KUNSTELJ, Ljubljana (SI); Barbara PODOBNIK, Ljubljana (SI)

(72) Inventors: Simona JEVSEVAR, Ljubljana (SI); Menci KUNSTELJ, Ljubljana (SI); Barbara PODOBNIK, Ljubljana (SI)

(73) Assignee: NOVARTIS AG, Basel (CH)

(21) Appl. No.: 14/567,430

(22) Filed: Dec. 11, 2014

Related U.S. Application Data
(60) Provisional application No. 61/915,090, filed on Dec. 12, 2013.

(51) Int. Cl. A61K 47/48; A61K 38/18

(52) U.S. Cl. CPC A61K 47/48215; A61K 38/18

(57) ABSTRACT

This invention is in the field of protein pegylation. In particular, it relates to a method for pegylating therapeutic proteins. The invention also relates to the use of such pegylated therapeutic polypeptides for treating muscle diseases and disorders.

### FIELD OF THE INVENTION

This invention is in the field of protein pegylation. In particular, it relates to pegylated therapeutic proteins, like pegylated IGF-1 polypeptides and variants thereof.

### BACKGROUND OF THE INVENTION

Covalent modification of proteins with poly(ethylene glycol) (PEG) has proven to be a useful method to extend the circulating half-lives of proteins in the body (Hershfield, M. S., et al., N. Engl. J. Med. 316 (1987) 589-596; Meyers, F. J., et al., Clin. Pharmacol. Ther. 49 (1991) 307-313; Delgado, C, et al., Crit. Rev. Ther. Drug). This process is known as PEGylation and it is the most established and successfully used technique to improve therapeutic values of pharmaceutically active proteins (Roberts et al, Chemistry for peptide and protein PEGylation. 2002, Advanced Drug Delivery Reviews 54 459-476; Caliceti P. et al, 2003). Attachment of PEG chains to the protein increases its molecular weight and hydrodynamic radius resulting in significant in vivo half-life extension. The PEG chains wrapped around the protein have a shielding effect on the protein and thus proteolytic degradation and immunogenicity of the conjugate are lowered. PEGylation also changes biodistribution properties and significantly increases solubility of hydrophobic proteins (Caliceti, P. and Veronese, F. M. 2003. Pharmacokinetic and biodistribution properties of poly(ethylene glycol)-protein conjugates. Adv. Drug Deliv. Rev., 55, 1261, Roberts et al, Chemistry for peptide and protein PEGylation. 2002, Advanced Drug Delivery Reviews 54 459-476)

Coupling chemistry achieved by using N-hydroxysuccinimmidyl ester of methoxypolyethyleneglycol (PEG-NHS) can lead to conjugation on different lysine residues resulting in complex mixture of positional isoforms and multiPEGylated forms. Several conjugates which have already been used in therapy for several years (PEG-Intron®, Pegasys®, MIRCERA®, Somavert®) are results of random PEGylation chemistry.

The major drawback of random PEGylation is lack of homogeneity of the final product. Consequently difficulties with reproducibility of the reaction and challenges with analytical characterization of the final product are expected.

Insulin-like growth factors (IGFs) are part of a complex system that cells use to communicate with their physiologic environment. This complex system (often referred to as the insulin-like growth factor axis) consists of two cell-surface receptors (IGF-1R and IGF-2R), two ligands (IGF-1 and IGF-2), a family of six high-affinity IGF-binding proteins (IGFBP 1-6), and associated IGFBP degrading enzymes (proteases). This system is important not only for the regulation of normal physiology but also for a number of pathological states (Glass, Nat Cell Biol 5:87-90, 2003).

The IGF axis has been shown to play roles in the promotion of cell proliferation and the inhibition of cell death (apoptosis). IGF-1 is mainly secreted by the liver as a result of stimulation by human growth hormone (hGH). Almost every cell in the human body is affected by IGF-1, especially cells in muscles, cartilage, bones, liver, kidney, nerves, skin and lungs. In addition to the insulin-like effects, IGF-1 can also regulate cell growth. IGF-1 and IGF-2 are regulated by a family of gene products known as the IGF-binding proteins. These proteins help to modulate IGF action in complex ways that involve both inhibiting IGF action by preventing binding to the IGF receptors as well as promoting IGF action through aiding delivery to the receptors and increasing IGF half-life in the blood stream. There are at least six characterized binding proteins (IGFBP1-6).

In its mature form, human IGF-1, also called somatomedin, is a small protein of 70 amino acids that has been shown to stimulate growth of a wide range of cells in culture. The IGF-1 protein is initially encoded by three known splice variant mRNAs. The open reading frame of each mRNA encodes a precursor protein containing the 70 amino acid IGF-1 (SEQ ID NO: 1) and a particular E-peptide at the C-terminus, depending on the particular IGF-1 mRNA. These E-peptides have been termed the Ea (rsvraqrhtdmpktqkevhlknasrgsagnknyrm; SEQ ID NO: 2), Eb (rsvraqrhtdmpktqkyqppstnkntksqrrkgwpkthpggeqkegteaslqirgkkkeqrreigsrnaecrgkkgk; SEQ ID NO: 3) and Ec (rsvraqrhtdmpktqkyqppstnkntksqrrkgstfeerk; SEQ ID NO: 4) peptides and range from 35 to 87 amino acids in length and encompass a common sequence region at the N-terminus and a variable sequence region at the C-terminus. For example, the wild-type open reading frame for the IGF-1-Ea encodes a polypeptide of 135 amino acids including the leader sequence and a polypeptide of 105 amino acids without the leader sequence (gpetlcgaelvdalqfvcgdrgfyfnkptgygsssrrapqtgivdeccfrscdIrrlemycaplkpaksarsvraqrhtdmpktqkevhlknasrgsagnknyrm; SEQ ID NO: 5). In physiological expression, the E-peptides are cleaved off of the precursor by endogenous proteases to yield the mature 70 amino acid IGF-1. The availability and half-life of IGF-1 in human serum is mainly influenced and modulated by proteases and IGF-1 binding proteins (IGFBP's). IGFBP's can either inhibit or potentiate IGF-1 activities (Oh Y, et al., Characterization of the affinities of insulin-like growth factor (IGF)-binding proteins 1-4 for IGF-I, IGF-II, IGF-I/insulin hybrid, and IGF-I analogs. Endocrinology. 1993 March; 132(3):1337-44). Strategies to increase the half-life of IGF-1 have been described in the prior art. Strategies that have been contemplated are

(i) the production of IGF-1 variants comprising specific mutations aiming to prevent the cleavage of IGF-1 in human serum by serine proteases, or to alleviate the negative impact of IGF-1 binding proteins on the availability or serum half-life of IGF-1 (WO200040613, WO05033134, WO2006074390, WO2007146689);
(ii) the production of IGF-1 fusion proteins, wherein the mature IGF-1 protein is fused to a human immunoglobulin Fc region (WO2005033134, WO200040613);
(iii) the use of IGF-1 precursor proteins wherein cleavage of the E-peptide from IGF-1 by a protease is reduced by modification of the precursor protein (WO2007146689);
(iv) combinations of the above described strategies ((i)/(ii) WO05033134, (i)/(ii) WO200040613, (i)/(iii) WO2007146689), and
(v) production of pegylated IGF-1 variants (WO2009121759, WO2008025528 and WO2006066891).

As IGF-1 suffers from poor pharmacokinetic properties (short elimination half-life), PEGylated versions of IGF variants were prepared. The preparation of PEGylated version of IGF-1 variant for the treatment of neuromuscular disorders was described in WO2008025528, WO2009121759 A2 and WO2006066891. Usually PEG is attached to amino groups of the protein. However, a major limitation of this amino pegylation approach is that proteins typically contain a considerable amount of lysine residues and therefore the poly(ethylene glycol) groups are attached to the protein in a non-specific manner. Pegylation of amino residue required for biological activity (e.g. residues near or at the active site of the protein) can result in low specific activity or inactivation of the protein.

To avoid some of the above described drawbacks WO2006066891 describes the use of conjugates consisting of IGF-1 variants and one or two poly(ethylene glycol) group(s), characterized in that said IGF-1 variant has been mutated at up to three amino acid at positions 27, 37, 65, 68 of the wild-type IGF-I amino acid sequence. However, each mutation introduced into a protein in order to minimize random pegylation, at the same time increases the risk of immunogenicity. Hence, as less as possible mutations are preferred when developing a therapeutic protein.

WO 2008025528 discloses the preparation of recombinant human IGF-I fusion proteins, wherein said fusion proteins comprise amino acid substitutions at positions lysine 27, 65 and/or 68. The process described in WO2008025528 allows the preparation of recombinant human IGF-I muteins which do not bear N-terminal PEGylation. The PEGylation reagent used in WO2006066891 and WO2008025528 was the N-hydroxysuccinimmidyl ester of methoxypolyethyleneglycol (PEG-NHS), which leads to randomly pegylated proteins. To avoid N-terminal PEGylation and the creation of positional isomers all lysine residues except one were replaced by polar amino acids and a propeptide was attached to the N-terminus. In the first step the IGF-1 mutein was PEGylated and afterwards the propeptide was cleaved from the IGF-1 with IgA protease leaving the IGF-1 mutein PEGylated only at a single lysine residue.

Reductive alkylation using methoxypolyethyleneglycol propionaldehyde (PEG-CHO) reagent is generally recognized as a site specific PEGylation method (Roberts et al, Chemistry for peptide and protein PEGylation. 2002, Advanced Drug Delivery Reviews 54 459-476). The N-terminal PEGylation was described in the Amgene related patent family (U.S. Pat. No. 7,090,835 B2, U.S. Pat. No. 6,956,027 B2, EP 0 822199B1). The reaction of reductive alkylation was performed under acidic conditions, at pH of 5.0 (EP 0822199 B1). Generally, it is believed, that a key property of PEG-CHO reagent is that under acidic conditions (approximately pH 5), aldehyde group is largely selective for the N-terminal α-amine because of the lower pKa of the α-amine compared to other nucleophiles (Kinstler et al., 2002, Molineux, 2004).

Selectivity of N-terminal PEGylation is usually significantly reduced for the proteins with highly exposed and highly reactive lysine's bringing a higher degree of complexity in the engineering process which is accompanied with longer development times and higher costs.

The various mutations in the IGF-1 precursor variants consisting of the IGF-1 mature protein (somatomedin) and an E-peptide as described in WO2007146689 allowed the tailoring of IGF-1 variants to achieve improved therapeutic effects. The mutations resulted in stabilized molecules that are metabolized more slowly by the body and therefore have a longer half-life than mature IGF-1 peptide. The slower clearance of the IGF-1 variants described in WO2007146689 from the body resulted in an improved efficacy compared to wild type IGF-1. However, the IGF-1 precursor variants disclosed in WO2007146689 possesses a high number of surface exposed lysine residues and PEGylation of said IGF-1 precursor variants results in formation of a mixture of monopegylated positional isoforms and pegylated proteins of different molecular weight. Although chromatographic purification methods could be applied to obtain a homogenous product, the separation of PEGylated mixtures is technically challenging since physicochemical characteristics of the forms are too similar. This results in low final process yields. Consequently, there was a need to develop an improved process which allows more selective PEGylation of IGF-1 precursor proteins.

### SUMMARY OF THE INVENTION

A first subject matter of the disclosure relates to a process for preparing a composition of a pegylated therapeutic protein, wherein in said composition at least 61 percent of the mono pegylated therapeutic protein fraction comprised in said composition is N-terminally mono-pegylated therapeutic protein, comprising the steps of reacting in an aqueous medium a therapeutic protein with a water-soluble polyethylene glycol under conditions of a reductive alkylation, characterized in that the pegylation reaction is performed within a pH range of about 6.5 to 7.5, to provide the composition of the pegylated therapeutic protein.

An additional embodiment of the disclosure relates to a process for preparing a composition of a mono pegylated therapeutic protein, wherein in said composition at least 65 percent of the mono pegylated protein fraction comprised in said composition is N-terminally mono-pegylated therapeutic protein, comprising the steps of

(a) reacting in an aqueous medium the therapeutic proteins with a water-soluble polyethylene glycol under conditions of a reductive alkylation, characterized in that the coupling reaction is performed within a pH range of about 6.5 to 7.5,
(b) performing a cation exchange chromatography step with the compositions obtained in step (a),
(c) obtaining elution fractions of the exchange chromatography step (b), and
(d) pool those fractions containing the mono-pegylated therapeutic proteins.

An additional subject matter of the disclosure relates to a process for preparing a composition of pegylated human IGF-1 precursor proteins, wherein in said composition at least 61% of the mono-pegylated human IGF1-precursor proteins comprised in said composition are N-terminally mono-pegylated IGF-1 precursor proteins, comprising the steps of reacting in an aqueous medium an IGF-1 precursor protein with a water-soluble polyethylene glycol under conditions of a reductive alkylation, characterized in that the coupling reaction is performed within a pH range of about 6.5 to 7.5.

In a particular embodiment the disclosure relates to a process for preparing a composition of mono pegylated human IGF-1 precursor proteins, wherein said composition comprises at least 65 percent of N-terminally mono-pegylated IGF-1 precursor protein, comprising the steps of
(a) reacting in an aqueous medium an IGF-1 precursor protein with a water-soluble polyethylene glycol under conditions of a reductive alkylation, characterized in that the coupling reaction is performed within a pH range of about 6.5 to 7.5,
(b) performing a cation exchange chromatography step with the compositions obtained in step (a),
(c) obtaining elution fractions of the exchange chromatography step (b), and
(d) pool those fractions containing the mono-pegylated IGF-1 precursor protein isoforms.

In a certain embodiment the disclosure relates to the above described processes characterized in that the pegylation (coupling reaction) is performed in the presence of α-cyclodextrine (α-CD)

In a particular embodiment of the disclosure, the above described pegylation reaction is performed at a pH of about 6.5. In a particular embodiment of the disclosure the PEG used in the above described processes is linear PEG having an overall molecular weight of from 20 to 100 kDa (kilo dalton). Consequently, in one embodiment of the disclosure the linear PEG used in the above described processes has an overall molecular weight of about 30 kDa. Alternatively, the PEG used in the above described method can be branched. In a particular embodiment of the disclosure the exchange chromatography step applied in the above described processes is a cation exchange chromatography (CEX) step. Likewise, the disclosure relates to the above described processes, further comprising the additional steps of

b) Ultrafiltration (UF)/diafiltration (DF) concentration and buffer exchange,
c) final filtration and filling.

In a particular embodiment of the disclosure the IGF1 precursor protein being pegylated according to above described processes is a human IGF-1Ea peptide precursor protein wherein amino acid E3 is deleted, amino acid R37 is substituted by alanine and the amino acids R71 and S72 are deleted, and wherein the numbering of the amino acids corresponds to SEQ ID NO: 5.

In one embodiment of the disclosure the IGF1 precursor protein being pegylated according to the above described processes is a human IGF-1Ea peptide precursor protein comprising the amino acid sequence of SEQ ID NO: 55.

Consequently, in an additional embodiment of the disclosure the IGF1 precursor protein being pegylated according to the above described processes is a human IGF-1Ea peptide precursor protein consisting of the amino acid sequence of SEQ ID NO: 55.

In yet another embodiment the disclosure relates to a composition of a pegylated therapeutic protein produced according to the above disclosed processes, wherein in said composition at least 61 percent of the mono pegylated therapeutic protein fraction comprised in said composition is N-terminally mono-pegylated therapeutic protein.

Additionally, the disclosure relates to a composition of a mono-pegylated therapeutic protein produced according to the above described processes, wherein in said composition at least 65% of the therapeutic protein fraction comprised in said composition is N-terminally mono-pegylated therapeutic protein.

Additionally, one embodiment of the disclosure relates to a composition of a pegylated therapeutic protein obtained by the above described processes, wherein in said composition at least 61 percent of the mono pegylated therapeutic protein fraction comprised in said composition is N-terminally mono-pegylated therapeutic protein.

Furthermore, in one embodiment of the disclosure relates to a composition of a pegylated therapeutic protein obtainable by the above described processes, wherein in said composition at least 61 percent of the mono pegylated therapeutic protein fraction comprised in said composition is N-terminally mono-pegylated therapeutic protein.

In a particular embodiment, the disclosure relates to a composition of a mono-pegylated therapeutic protein obtainable by the above described processes, wherein in said composition at least 65% of the pegylated therapeutic protein fraction comprised in said composition is N-terminally mono-pegylated therapeutic protein.

Additionally, one embodiment of the disclosure relates to a composition of a mono-pegylated therapeutic protein obtained by the above described processes, wherein in said composition at least 65% of the mono pegylated therapeutic protein fraction comprised in said composition is N-terminally mono-pegylated therapeutic protein.

In yet another embodiment the disclosure relates to a mono pegylated IGF-1 composition produced according to the above disclosed processes, wherein in said composition at least 61 percent of the mono pegylated IGF-1 protein fraction comprised in said composition is N-terminally mono-pegylated IGF-1 precursor protein.

Additionally, the disclosure relates to a pegylated IGF-1 composition produced according to the above described processes, wherein in said composition at least 70% of the pegylated IGF-1 precursor protein fraction comprised in said composition is a mixture of N-terminally and lysine residue mono-pegylated IGF-1 precursor protein.

Furthermore, in one embodiment the disclosure relates to a mono pegylated IGF-1 composition obtainable by the above described processes, wherein in said composition at least 61 percent of the mono pegylated IGF-1 protein fraction comprised in said composition is N-terminally mono-pegylated IGF-1 precursor protein.

In a particular embodiment, the disclosure relates to a pegylated IGF-1 composition obtainable by the above described processes, wherein in said composition at least 70% of the pegylated IGF-1 precursor protein fraction comprised in said composition is a mixture of N-terminally and lysine residue mono-pegylated IGF-1 precursor protein.

Additionally, one embodiment of the disclosure relates to a mono pegylated IGF-1 composition obtained by the above described processes, wherein in said composition at least 61 percent of the mono pegylated IGF-1 protein fraction comprised in said composition is N-terminally mono-pegylated IGF-1 precursor protein.

In another embodiment the disclosure relates to a pegylated IGF-1 composition obtained by the above described processes, wherein in said composition at least 70% of the pegylated IGF-1 precursor protein fraction comprised in said composition is a mixture of N-terminally and lysine residue mono-pegylated IGF-1 precursor protein.

In a particular embodiment of the disclosure, the IGF1 precursor protein comprised in the above disclosed compositions is a human IGF-1Ea peptide precursor protein wherein amino acid E3 is deleted, amino acid R37 is substituted by alanine and the amino acids R71 and S72 are deleted, and wherein the numbering of the amino acids corresponds to SEQ ID NO: 5.

Furthermore, the disclosure relates to the above described compositions, wherein the IGF1 precursor protein is a human IGF-1Ea peptide precursor protein comprising the amino acid sequence of SEQ ID NO: 55.

Consequently, the disclosure relates to the above described compositions, wherein the IGF1 precursor protein is a human IGF-1Ea peptide precursor protein consisting of the amino acid sequence of SEQ ID NO: 55.

In yet another embodiment, the disclosure related to the above described compositions in a pharmaceutically acceptable form for use as a medicament.

The disclosure furthermore provides the above described pharmaceutical compositions for use in therapy.

In another embodiment of the disclosure, the above mentioned therapeutic use is the treatment of a muscle disorder in a patient in need thereof. In a particular embodiment of the disclosure, the therapeutic use is the treatment of burn patients suffering from loss of lean body mass and/or muscle wasting or the treatment of COPD patients, or the treatment of Spinal and Bulbar Muscular Atrophy (SBMA or Kennedy disease) patients, or the treatment of chronic kidney disease patients. Accordingly, the disclosure provides a pharmaceutical composition as described above for use in the treatment of a disease or condition selected from the group consisting of burns in combination with loss of lean body mass and/or muscle wasting, chronic obstructive pulmonary disease (COPD), Spinal and Bulbar Muscular Atrophy (SBMA, Kennedy disease) and chronic kidney disease.

In an additional embodiment of the disclosure, the muscle disorder described above is muscle atrophy. In some aspects of the disclosure, the therapeutic use is the treatment of obesity-associated sarcopenia, sarcopenia, and diabetes-associated muscle atrophy. Accordingly, the disclosure provides a pharmaceutical composition as described above for use in the treatment of a disease or condition selected from the group consisting of muscle atrophy, obesity-associated sarcopenia, sarcopenia, and diabetes-associated muscle atrophy

The disclosure furthermore provides for a method of treating a muscle disorder in a patient in need thereof, the method comprising administering a therapeutically effective amount of the above described compositions.

Accordingly, in one particular embodiment, the disclosure relates to a method of treating burn patients suffering from loss of lean body mass and/or muscle wasting, or a method of treating chronic obstructive pulmonary disease (COPD) patients, or a method of treating Kennedy disease patients, or a method of treating chronic kidney disease patients, comprising administering a therapeutically effective amount of the above described compositions.

Furthermore, the disclosure provides a method of treating a muscle disorder in a patient in need thereof, wherein the muscle disorder is a muscle atrophy selected from the group consisting of obesity-associated sarcopenia, sarcopenia, and diabetes-associated muscle atrophy, comprising administering a therapeutically effective amount of the above described compositions.

Certain embodiments of the disclosure are described in the following aspects:
1. A process for preparing a composition of a pegylated therapeutic protein, wherein in said composition at least 61 percent of the mono pegylated therapeutic protein fraction comprised in said composition is N-terminally mono-pegylated therapeutic protein, comprising the steps of reacting in an aqueous medium a therapeutic protein with a water-soluble polyethylene glycol under conditions of a reductive alkylation, characterized in that the reaction is performed within a pH range of about 6.5 to 7.5, to provide the composition of the pegylated therapeutic protein.
2. A process for preparing a composition of a mono pegylated therapeutic protein, wherein in said composition at least 65 percent of the mono pegylated protein fraction comprised in said composition is N-terminally mono-pegylated therapeutic protein, comprising the steps of
   (a) reacting in an aqueous medium the therapeutic proteins with a water-soluble polyethylene glycol under conditions of a reductive alkylation, characterized in that the coupling reaction is performed within a pH range of about 6.5 to 7.5,
   (b) performing a chromatography step with the compositions obtained in step (a), to provide the composition of the mono pegylated therapeutic protein.
3. A process according to embodiment 2, wherein the chromatography step is a cation exchange chromatography step comprising pooling those fractions containing the N-terminally mono-pegylated therapeutic protein, to provide the composition of the mono pegylated therapeutic protein.
4. A process for preparing a composition of according to any of the embodiments 1-3, wherein the therapeutic protein is a human IGF-1 precursor protein or a variant thereof.
5. A process according to any of the preceding embodiments, characterized in that the pegylation reaction is performed in the presence of α-cyclodextrine (α-CD).
6. A process according to any of the preceding embodiments, characterized in that the PEG has an overall molecular weight of from 20 to 100 kDa.
7. A process according to embodiment 6, characterized in that the PEG has an overall molecular weight of about 30 kDa.
8. The process according to embodiment 2 and 4-7, wherein the exchange chromatography step is a cation exchange chromatography (CEX).
9. A process according to embodiment 2 and 4-8, further comprising the additional steps of
   b) Ultrafiltration (UF)/diafiltration (DF) concentration and buffer exchange,
   c) final filtration.
10. The process according to any of the embodiments 3-9, wherein the IGF1 precursor protein is a human IGF-1Ea peptide precursor protein wherein amino acid E3 is deleted, amino acid R37 is substituted by alanine and the amino acids R71 and S72 are deleted, and wherein the numbering of the amino acids corresponds to SEQ ID NO: 5.
11. The process according to embodiment 10, wherein the IGF1 precursor protein is a human IGF-1Ea peptide precursor protein consisting of SEQ ID NO: 55.
12. A composition produced according to the processes of embodiments 1, 5, 6 and 7, wherein in said composition at least 61 percent of the mono pegylated therapeutic protein fraction comprised in said composition is N-terminally mono-pegylated therapeutic protein.
13. A composition of a mono-pegylated therapeutic protein produced according to the processes of embodiments 2 and 5-9, wherein in said composition at least 65% of the therapeutic protein fraction comprised in said composition is N-terminally mono-pegylated therapeutic protein.
14. A composition of a pegylated therapeutic protein obtainable by the processes of embodiments 1, 5, 6 and 7, wherein in said composition at least 61 percent of the mono pegylated therapeutic protein fraction comprised in said composition is N-terminally mono-pegylated therapeutic protein.
15. A composition of a mono-pegylated therapeutic protein obtainable by the processes of embodiments 2 and 5-9, wherein in said composition at least 65% of the pegylated therapeutic protein fraction comprised in said composition is N-terminally mono-pegylated therapeutic protein.
16. A composition of a pegylated therapeutic protein obtained by the processes of embodiments 1, 5, 6 and 7, wherein in said composition at least 61 percent of the mono pegylated therapeutic protein fraction comprised in said composition is N-terminally mono-pegylated therapeutic protein.
17. A composition of a mono-pegylated therapeutic protein obtained by the processes of embodiments 2 and 5-9, wherein in said composition at least 65% of the mono pegylated therapeutic protein fraction comprised in said composition is N-terminally mono-pegylated therapeutic protein.
18. A composition produced according to the processes of embodiments 10 and 11, wherein in said composition at least 70% of the pegylated IGF-1 precursor protein fraction comprised in said composition is a mixture of N-terminally and lysine residue mono-pegylated IGF-1 precursor protein.
19. A composition obtainable by the processes of embodiments 10 and 11, wherein in said composition at least 61 percent of the mono pegylated IGF-1 protein fraction comprised in said composition is N-terminally mono-pegylated IGF-1 precursor protein.
20. A composition obtainable by the processes of embodiments 10 and 11, wherein in said composition at least 70% of the pegylated IGF-1 precursor protein fraction comprised in said composition is a mixture of N-terminally and lysine residue mono-pegylated IGF-1 precursor protein.
21. A composition obtained by the processes of claims 10 and 11, wherein in said composition at least 61 percent of the mono pegylated IGF-1 protein fraction comprised in said composition is N-terminally mono-pegylated IGF-1 precursor protein.
22. A composition obtained by the processes of embodiments 10 and 11, wherein in said composition at least 70% of the pegylated IGF-1 precursor protein fraction comprised in said composition is a mixture of N-terminally and lysine residue mono-pegylated IGF-1 precursor protein.
23. A composition according to any of the embodiments 18 to 22, wherein the IGF1 precursor protein is a human IGF-1Ea peptide precursor protein wherein amino acid E3 is deleted, amino acid R37 is substituted by alanine and the amino acids R71 and S72 are deleted, and wherein the numbering of the amino acids corresponds to SEQ ID NO: 5.
24. A composition according to embodiment 23, wherein the IGF1 precursor protein is a human IGF-1Ea peptide precursor protein comprising the amino acid sequence of SEQ ID NO: 55.
25. Composition according to any of the embodiments 12-24 in a pharmaceutically acceptable form for use as a medicament.
26. A pharmaceutical composition comprising a pegylated therapeutic protein obtained by the processes of embodiments 1-11 for use in therapy.
27. The pharmaceutical composition of embodiment 25 or 26 for use in the treatment of a muscle disorder in a patient in need thereof.
28. The pharmaceutical composition of embodiment 25 or 26 for use in the treatment of burn patients suffering from loss of lean body mass and/or muscle wasting.
29. The pharmaceutical composition of embodiment 25 or 26 for use in the treatment of chronic obstructive pulmonary disease (COPD) patients.
30. The pharmaceutical composition of embodiment 25 or 26 for use in the treatment of Spinal and Bulbar Muscular Atrophy (SBMA or Kennedy disease) patients.
31. The pharmaceutical composition of embodiment 25 or 26 for use in the treatment of chronic kidney disease patients.
32. The pharmaceutical composition of embodiment 27, wherein the muscle disorder is muscle atrophy.
33. The pharmaceutical composition of embodiment 32, wherein the muscle atrophy is selected from the group consisting of obesity-associated sarcopenia, sarcopenia, and diabetes-associated muscle atrophy.
34. A method of treating a muscle disorder in a patient in need thereof, the method comprising administering a therapeutically effective amount of the composition of embodiment 25 or 26 to a subject.
35. A method of treating burn patients suffering from loss of lean body mass and/or muscle wasting, the method comprising administering a therapeutically effective amount of the composition of embodiment 25 or 26 to a subject.
36. A method of treating chronic obstructive pulmonary disease (COPD) patients, the method comprising administering a therapeutically effective amount of the composition of embodiment 25 or 26 to a subject.
37. A method of treating Spinal and Bulbar Muscular Atrophy (SBMA or Kennedy disease) patients, the method comprising administering a therapeutically effective amount of the composition of embodiment 25 or 26 to a subject.
38. A method of treating chronic kidney disease patients, the method comprising administering a therapeutically effective amount of the composition of embodiment 25 or 26 to a subject.
39. A method according to embodiment 32, wherein the muscle disorder is a muscle atrophy selected from the group consisting of obesity-associated sarcopenia, sarcopenia, and diabetes-associated muscle atrophy.

### BRIEF DESCRIPTION OF THE DRAWINGS

FIG. 1: Process flow diagram describing the process for preparation of pegylated IGF-1Ea. The process is composed of pegylation reaction, cation-exchange purification step (CEX), buffer exchange and concentrating step (UF/DF) and final filtration and filling step.

FIG. 2: Preparative CEX chromatogram for partial separation of monoPEG-IGF1 isoforms. 1: N-terminally pegylated IGF-1Ea form; 2, 3 and 4: Lys-pegylated IGF-1Ea forms. Numbered arrows indicate the position of the pooled fractions on the chromatogram. Pools designated as 1, 2, 3, and 4 were prepared with pooling of fractions eluted at time marked on chromatogram with arrows.

FIG. 3: CE-HPLC analysis of pooled material from preparative CEX isolation of monoPEG-IGF1 isoforms. N: N-terminally pegylated IGF-1Ea form; lysine 1, lysine 2, lysine 2a and lysine 3: Lys-pegylated IGF-1Ea forms.

FIG. 4: Preparative CEX chromatogram of separation on Toyopearl SP650S.

FIG. 5: CEX chromatogram of separation on SP Sepharose HP.

### GENERAL DEFINITIONS

In order that the present invention may be more readily understood, certain terms are first defined. Additional definitions are set forth throughout the detailed description.

About: the term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 5 percent up or down (higher or lower). For example the term at a pH of about 6.5 would include a pH range of 6.3 to 6.8. As used herein, and if not otherwise specified, the word “or” means any one member of a particular list. The phrase within a pH range of about 6.5 to 7.5 would include a pH range of 6.3 to 7.9

Comprising: the term “comprising” means “including” e.g. a composition “comprising” X may consist exclusively of X or may include something additional e.g. X+Y.

An IGF-1 protein variant: is a protein that differs by at least one amino acid from the IGF-1 wild-type sequence, wherein the term “wild-type sequence” refers to a polypeptide or gene sequence available in at least one naturally occurring organism or a polypeptide or gene sequence that has not been changed, mutated, or otherwise manipulated by man (the human IGF-1 wildtype sequence if specified by the protein sequence of SEQ ID NO: 1). An IGF-1 variant is also the IGF-1 precursor protein or the pro-IGF-1 protein comprising a peptide leader sequence. An IGF-1 variant as described above retains its biological activity in the sense that such a protein can be considered as a functional equivalent of the wildtype IGF-1. A functional equivalent of the IGF-1 wildtype protein has specific affinities to the IGF-1 receptor protein.

Functional equivalents with regard to the IGF-1 protein have to be understood as IGF-1 proteins comprising natural or artificial mutation. Mutations can be insertions, deletions or substitutions of one or more nucleic acids that do not diminish the biological activity of the IGF-1 protein. Functional equivalents having an identity of at least 80%, preferably 85%, more preferably 90%, most preferably more than 95%, very especially preferably at least 98% identity—but less than 100% identity to the IGF-1 wildtype protein, e.g. the human IGF-1 protein SEQ ID NO: 1. In case of fusion proteins, the 100% identity shall be defined only on the basis of the IGF-1 part of such a fusion protein.

Insulin like growth factors (IGFs) are part of a complex system that cells use to communicate with their physiologic environment. This complex system (often referred to as the insulin-like growth factor axis) consists of two cell-surface receptors (IGF-1R and IGF-2R), two ligands (IGF-1 and IGF-2), a family of six high-affinity IGF-binding proteins (IGFBP 1-6), and associated IGFBP degrading enzymes (proteases). This system is important not only for the regulation of normal physiology but also for a number of pathological states (Glass, Nat Cell Biol 5:87-90, 2003). The IGF axis has been shown to play roles in the promotion of cell proliferation and the inhibition of cell death (apoptosis). IGF-1 is mainly secreted by the liver as a result of stimulation by human growth hormone (hGH). Almost every cell in the human body is affected by IGF-1, especially cells in muscles, cartilage, bones, liver, kidney, nerves, skin and lungs. In addition to the insulin-like effects, IGF-1 can also regulate cell growth. IGF-1 and IGF-2 are regulated by a family of gene products known as the IGF-binding proteins. These proteins help to modulate IGF action in complex ways that involve both inhibiting IGF action by preventing binding to the IGF receptors as well as promoting IGF action through aiding delivery to the receptors and increasing IGF half-life in the blood stream. There are at least six characterized binding proteins (IGFBP1-6). IGF-1 is used in a wide range of therapeutic applications. Mecasermin (brand name Increlex™) is a synthetic analog of IGF-1 which is approved for the treatment of growth failure. Several companies have evaluated IGF-1 in clinical trials for a variety of additional indications, including type 1 diabetes, type 2-diabetes, amyotrophic lateral sclerosis, severe burn injury and myotonic muscular dystrophy.

For the sake of clarity and consistency, the numbering of amino acid residues in IGF-1 precursor or mature proteins throughout this application and in the claims is based on the wild-type precursor protein sequence numbering of the human insulin-like growth factor 1 (somatomedin C), isoform CRA_c (accession no. EAW97697) without signal peptide (i.e. SEQ ID NO: 5).

PEG: when used in the context of this disclosure refers to polyethylene glycol.

PEG-CHO refers to methoxypolyethyleneglycol propionaldehyde reagent (e.g. SUNBRIGHT ME-300AL purchased from NOF Corporation, Japan or 30 kDa mPEG propionaldehyde purchased from Dr Reddy's (EU) Limited).

The term “higher PEG forms”, “higher PEGylated forms”, “higher pegylated variants” or “di-, tri- or higher PEGylated forms” is used to described proteins to which more than one PEG molecule has been attached (e.g. two, three or more PEG molecules) di-, tri- or higher PEGylated proteins. The term “mono-pegylated” is used to describe a situation in which to a given protein only a single PEG molecule has been attached during the pegylation process.

“PEGylation reaction”, PEGylation” or “pegylation process” refers to the process of covalent attachment of polyethylene glycol (PEG) polymer chains to another molecule, in the context of this invention, to a human IGF-1 precursor molecule.

Precursor: In the following, the term “precursor” when used in the context of the present invention shall refer to the precursor of the mature human IGF-1 protein without signal peptide, but including the Ea, Eb and Ec peptide, respectively.

Reductive alkylation: the term “a reductive alkylation” refers to a reaction where in the presence of a reducing agent a carbonyl group and an amino group are converted to an amine. This reaction has been known for many years and it is considered the most important way to make amines.

In the case of PEGylation PEG-CHO reagent under mild reducing conditions reacts with amino groups of proteins. The reaction proceeds in two steps, condensation and reduction. In the first step an unstable Schiff's base is formed, which is subsequently reduced to stable secondary amine bond between the reagent and protein:

### Step I: Condensation

CH₃O(CH₂CH₂O)ₙ—CH₂CH₂CHO+NH₂(N-term.)-Prot [symbol] CH₃O(CH₂CH₂O)ₙ—CH₂CH₂CH═N(Nterm.)-Prot

### Step II: Reduction

CH₃O(CH₂CH₂O)ₙ—CH₂CH₂CH═N(Nterm.)-Prot→CH₃O(CH₂CH₂O)ₙ—CH₂CH₂CH₂NH-Prot

For protein modification, a mild reducing agent, such as sodium cyanoborohydride is usually chosen to avoid simultaneous reduction of the native disulfide bonds in the protein.

Therapeutic protein: the term “therapeutic protein(s)” refers to protein(s) for use in human or veterinary therapy and may be intended for acute or chronic administration. In particular, a “therapeutic protein” is a protein used in the treatment of a mammal having a disease or pathological condition.

### DETAILED DESCRIPTION OF THE INVENTION

This invention describes the preparation and purification of PEGylated therapeutic proteins in a highly reproducible manner. The product is a mono-PEGylated protein with polyethylenglcol (PEG) attached to N-terminal or lysine amino groups.

The present invention provides a procedure for the PEGylation of therapeutic proteins with improved selectivity. The conditions used during PEGylation offer the ability to alter or adjust the profile of PEGylated therapeutic protein variants and also to reduce the amount of higher PEGylated protein variants. The parameter which directs the reaction toward desired conjugates is the pH of the PEGylation mixture. Surprisingly, addition of α-cyclodextrine (α-CD) in the PEGylation mixture significantly decreases reaction rate and consequently significantly reduces the formation of higher PEGylated forms. Furthermore α-CD enables better control of PEGylation reaction by slowing it down. The PEGylation conditions ensure high yields and high reproducibility of the process.

The disclosure relates to a purification/isolation process which employs chromatography steps (e.g. cation exchange chromatography (CEX)) in a way where a partial separation between lysine (Lys) and N-terminally PEGylated isoforms is achieved and enables control over the composition of PEGylated isoforms in final mono-PEGylated product.

In one embodiment the disclosure relates to a process for preparing a composition of a pegylated therapeutic protein, wherein in said composition at least 61 percent of the mono pegylated therapeutic protein fraction comprised in said composition is N-terminally mono-pegylated therapeutic protein, comprising the steps of reacting in an aqueous medium said therapeutic protein with a water-soluble polyethylene glycol under conditions of a reductive alkylation, characterized in that the coupling reaction is performed within a pH range of about 6.5 to 7.5.

An additional embodiment the disclosure relates to a process for preparing a composition of a mono pegylated therapeutic protein, wherein in said composition at least 65 percent of the mono pegylated protein fraction comprised in said composition is N-terminally mono-pegylated therapeutic protein, comprising the steps of

(a) reacting in an aqueous medium the therapeutic proteins with a water-soluble polyethylene glycol under conditions of a reductive alkylation, characterized in that the coupling reaction is performed within a pH range of about 6.5 to 7.5,
(b) performing an exchange chromatography step with the compositions obtained in step (a),
(c) obtaining elution fractions of the exchange chromatography step (b), and
(d) pool those fractions containing the mono-pegylated therapeutic proteins.

The disclosure also relates to a composition of a pegylated therapeutic protein produced according to the above described method, wherein in said composition at least 61 percent of the mono pegylated therapeutic protein fraction comprised in said composition is N-terminally mono-pegylated therapeutic protein.

Additionally, the disclosure relates to a composition of a mono-pegylated therapeutic protein produced according to the above described processes, wherein in said composition at least 65% of the therapeutic protein fraction comprised in said composition is N-terminally mono-pegylated therapeutic protein.

Additionally, one embodiment of the disclosure relates to a composition of a pegylated therapeutic protein obtained by the above described processes, wherein in said composition at least 61 percent of the mono pegylated therapeutic protein fraction comprised in said composition is N-terminally mono-pegylated therapeutic protein.

Furthermore, in one embodiment of the disclosure relates to a composition of a pegylated therapeutic protein obtainable by the above described processes, wherein in said composition at least 61 percent of the mono pegylated therapeutic protein fraction comprised in said composition is N-terminally mono-pegylated therapeutic protein.

In a particular embodiment, the disclosure relates to a composition of a mono-pegylated therapeutic protein obtainable by the above described processes, wherein in said composition at least 65% of the pegylated therapeutic protein fraction comprised in said composition is N-terminally mono-pegylated therapeutic protein.

Additionally, one embodiment of the disclosure relates to a composition of a mono-pegylated therapeutic protein obtained by the above described processes, wherein in said composition at least 65% of the mono pegylated therapeutic protein fraction comprised in said composition is N-terminally mono-pegylated therapeutic protein.

The therapeutic protein mentioned above can be a growth factor. Growth factors are known in the art and include, without being limited to, Adreno-medullin (AM), Angiopoietin (Ang), Autocrine motility factor, Bone morphogenetic proteins (BMPs), Brain-derived neurotrophic factor (BDNF), Epidermal growth factor (EGF), Erythropoietin (EPO), Fibroblast growth factor (FGF), Glial cell line-derived neurotrophic factor (GDNF), Granulocyte colony-stimulating factor (G-CSF), Granulocyte macrophage colony-stimulating factor (GM-CSF), Growth differentiation factor-9 (GDF9)

Hepatocyte growth factor (HGF), Hepatoma-derived growth factor (HDGF), Migration-stimulating factor, Myostatin (GDF-8), Nerve growth factor (NGF) and other neurotrophins, Platelet-derived growth factor (PDGF), Thrombopoietin (TPO), Transforming growth factor alpha (TGF-α), Transforming growth factor beta (TGF-β), Tumor_necrosis_factor-alpha (TNF-α), Vascular endothelial growth factor (VEGF), Wnt Signaling Pathway placental growth factor (PlGF), Foetal Bovine Somatotrophin (FBS), Interleukin 2—(IL2), IL3-, IL4-, IL5-, IL6-, IL7-growth factor.

Therefore, this invention also relates to the preparation and purification of PEGylated Insulin-like growth factor-1 or variants thereof like the Insulin-like growth factor-1 Ea variant (IGF-1Ea) in a highly reproducible manner. The product is a mono-PEGylated IGF-1 precursor protein (e.g. IGF-1Ea) with polyethylenglcol (PEG) attached to N-terminal or lysine amino groups.

The present invention provides a procedure for PEGylation of IGF-1 or variants thereof like the IGF-1Ea with improved selectivity. The conditions used during PEGylation offer the ability to alter or adjust the profile of PEGylated IGF-1 or variants thereof like the IGF-1Ea precursor protein and also to reduce the amount of higher PEGylated IGF-1Ea precursor protein forms. The parameter which directs the reaction toward desired conjugates is the pH of the PEGylation mixture. Surprisingly, addition of α-cyclodextrine (α-CD) in the PEGylation mixture significantly decreases reaction rate and consequently significantly reduces the formation of higher PEGylated IGF-1 forms or variants thereof like the IGF-1Ea precursor protein. Furthermore α-CD enables better control of PEGylation reaction by slowing it down. The PEGylation conditions ensure high yields and high reproducibility of the process.

The present invention also describes the purification/isolation process which employs chromatography steps (e.g. cation exchange chromatography (CEX)) in a way where a partial separation between lysine (Lys) and N-terminally PEGylated isoforms is achieved and enables control over the composition of PEGylated isoforms in final mono-PEGylated product.

Therefore, in one aspect, the invention provides a composition of pegylated human IGF-1 proteins or variants thereof like the IGF-1Ea precursor protein directly obtained from a pegylation process, wherein in said composition at least 61 percent, or at least 62 percent, or at least 63 percent, or at least 64 percent, or at least 65, or at least 66, or at least 67 percent of the mono pegylated hIGF-1 precursor protein fraction comprised in said composition is N-terminally mono-pegylated hIGF-1 protein, e.g. the IGF-1Ea precursor protein.

Additionally, the invention provides a composition of pegylated human IGF-1 proteins or variants thereof like the IGF-1Ea precursor protein directly obtained from a pegylation process, wherein in said composition at least 61 percent, or at least 62 percent, or at least 63 percent, or at least 64 percent, or at least 65, or at least 66, or at least 67 percent of the mono pegylated fraction of said composition is N-terminally mono-pegylated IGF-1 precursor protein and wherein the amount of higher pegylated IGF-1 precursor proteins is less than 20%, or is less than 19%, or is less than 18%, or is less than 17% of all pegylated IGF-1 proteins, e.g. the IGF-1Ea precursor protein.

The amount of mono-pegylated and higher pegylated IGF-1 proteins, e.g. the IGF-1Ea precursor protein, present in the reaction mixture resulting from the pegylation process can be determined by Reverse Phase HPLC methods (Park et al, Journal of Chromatography A, 1216, (2009), 7793-7797, Seely el al, BioPharm Int, (2005) 1-7) well known to the person skilled in the art.

The amount of N-terminally mono-pegylated IGF-1 proteins, e.g. the IGF-1Ea precursor protein, present in the reaction mixture resulting from the pegylation process can be determined by cation exchange chromatography HPLC (CE-HPLC) methods well known to the person skilled in the art (Wang et al, Advanced Drug Delivery Reviews, 17 (2002), 547-570, Monkarsh et al, Anal Biochem, 247, (1997), 434-440).

Consequently, the invention also provides a composition of pegylated human IGF-1 proteins, e.g. the IGF-1Ea precursor protein, directly obtained from a pegylation process, wherein said mono pegylated IGF-1 precursor protein fraction of said composition contains at least 66 percent of N-terminally mono-pegylated human IGF-1 precursor protein.

Additionally, the invention also provides a composition of pegylated human IGF-1 proteins, e.g. the IGF-1Ea precursor protein, directly obtained from a pegylation process, wherein said mono pegylated IGF-1 precursor protein fraction of said composition contains at least 66 percent of N-terminally mono-pegylated human IGF-1 precursor protein and wherein the amount of higher pegylated IGF-1 precursor proteins is less than 20% of all pegylated IGF-1 precursor proteins.

In a particular embodiment of the disclosure the PEG attached to the human IGF-1 precursor proteins comprised in the compositions described above is linear or branched and has an overall molecular weight of from 20 to 100 kDa, or from 20 to 80 kDa, or from 20 to 70 kDa, or from 20 to 60 kDa, or from 20 to 50 kDa. Accordingly, in one embodiment of the invention, the composition comprises human IGF-1 proteins, e.g. the IGF-1Ea precursor protein, pegylated with linear PEG having a molecular weight of about 30 kDa.

The pegylated human IGF-1 precursor molecule comprised in the composition of the invention can be the wildtype human IGF-1 precursor protein comprising the Ea, Eb, Ec peptide. Additionally, the pegylated human IGF-1 precursor molecule comprised in the composition of the invention can be a mutated version of the human IGF-1 precursor protein comprising the Ea, Eb, Ec peptide. Accordingly, the invention provides the above described composition comprising a pegylated human IGF-1Ea peptide precursor protein, wherein at least 61 percent, or at least 62 percent, or at least 63 percent, or at least 64 percent, or at least 65, or at least 66, or at least 67, or at least 68, or at least 69, or at least 70 percent or more of the mono pegylated human IGF-1Ea peptide precursor protein fraction are mono-pegylated at the N-terminus, and wherein one or more amino acids at the positions selected from group consisting of G1, P2, E3, R36, R37, G42, K68, S69, A70, R71, S72, R74, R77, G96, S97, A98, G99, N100, K101, N102, Y103, Q104 and/or M105 have been mutated and or deleted, wherein the numbering of the amino acids corresponds to SEQ ID NO: 5.

Furthermore, the invention provides a composition of a mono-pegylated human IGF-1Ea peptide precursor protein, wherein said composition comprises at least 70 percent, or at least 80 percent, or at least 90 percent, or at least 91 percent, or at least 92 percent, or at least 93 percent, or at least 94 percent, or at least 95 percent, or at least 96 percent, or at least 97 percent, or more of mono-pegylated IGF-1 precursor protein, and wherein one or more amino acids at the positions selected from group consisting of G1, P2, E3, R36, R37, G42, K68, S69, A70, R71, S72, R74, R77, G96, S97, A98, G99, N100, K101, N102, Y103, Q104 and/or M105 have been mutated and or deleted, wherein the numbering of the amino acids corresponds to SEQ ID NO: 5.

Examples of such molecules include, but are not limited to, the following polypeptide variants:

A polypeptide comprising a human IGF-1Ea-peptide precursor protein wherein the amino acid(s)

(1) G1, P2, E3 are deleted, amino acid R36 is substituted or deleted and the amino acids R71 and S72 are deleted.
(2) G1, P2, E3 are deleted, amino acid R36 is substituted by glutamine (Q) and the amino acids R71 and S72 are deleted.
(3) G1, P2, E3 are deleted, amino acid R37 is substituted or deleted and the amino acids R71 and S72 are deleted.
(4) G1, P2, E3 are deleted, amino acid R37 is substituted by glutamic acid (E) and the amino acids R71 and S72 are deleted.
(5) G1, P2, E3 are deleted, amino acid R37 is substituted by alanine and the amino acids R71 and S72 are deleted.
(6) G1, P2, E3 are deleted, amino acid R37 is substituted by proline (P) and the amino acids R71 and S72 are deleted.
(7) G1, P2, E3 are deleted, amino acids R36 and R37 are substituted or deleted and the amino acids R71 and S72 are deleted.
(8) G1, P2, E3 are deleted, amino acid R36 and R37 are both substituted by glutamine (Q) and the amino acids R71 and S72 are deleted.
(9) G1, P2, E3 are deleted, amino acid R36 is substituted by glutamine (Q), R37 is substituted by alanine and the amino acids R71 and S72 are deleted.
(10) G1, P2, E3 are deleted, amino acid R36 is substituted or deleted and the amino acids R71 and S72 are deleted and amino acid R77 is mutated to glutamine (Q).
(11) G1, P2, E3 are deleted, amino acid R36 is substituted or deleted and the amino acids R71 and S72 are deleted and amino acids R74 and R77 are mutated to glutamine (Q).
(12) G1, P2, E3 are deleted, amino acid R36 is substituted or deleted and the amino acids R71 and S72 are deleted and amino acids R74, R77 and Q104 are mutated to glutamine (Q).
(13) G1, P2, E3 are deleted, amino acid R36 is substituted by glutamine (Q) and the amino acids R71 and S72 are deleted and amino acid R74 is mutated to glutamine (Q).
(14) G1, P2, E3 are deleted, amino acid R36 is substituted by glutamine (Q) and the amino acids R71 and S72 are deleted and amino acids R74 and R77 are mutated to glutamine (Q).
(15) G1, P2, E3 are deleted, amino acid R36 is substituted by glutamine (Q) and the amino acids R71 and S72 are deleted and amino acids R74, R77 and R104 are mutated to glutamine (Q).
(16) G1, P2, E3 are deleted, amino acid R37 is substituted by glutamic acid (E) and the amino acids R71 and S72 are deleted and amino acid R77 is mutated to glutamine (Q).
(17) G1, P2, E3 are deleted, amino acid R37 is substituted by glutamic acid (E) and the amino acids R71 and S72 are deleted and amino acids R74 and R77 are mutated to glutamine (Q).
(18) G1, P2, E3 are deleted, amino acid R37 is substituted by glutamic acid (E) and the amino acids R71 and S72 are deleted and amino acids R74, R77 and R104 are mutated to glutamine (Q).
(19) G1, P2, E3 are deleted, amino acid R37 is substituted by alanine (A) and the amino acids R71 and S72 are deleted and amino acid R74 is mutated to glutamine (Q).
(20) G1, P2, E3 are deleted, amino acid R37 is substituted by alanine (A) and the amino acids R71 and S72 are deleted and amino acids R74 and R77 are mutated to glutamine (Q).
(21) G1, P2, E3 are deleted, amino acid R37 is substituted by alanine (A) and the amino acids R71 and S72 are deleted and amino acids R74, R77 and R104 are mutated to glutamine
(22) G1, P2, E3 are deleted, amino acid R37 is substituted by proline (P) and the amino acids R71 and S72 are deleted and amino acid R74 is mutated to glutamine (Q).
(23) G1, P2, E3 are deleted, amino acid R37 is substituted by proline (P) and the amino acids R71 and S72 are deleted and amino acids R74 and R77 are mutated to glutamine (Q).
(24) G1, P2, E3 are deleted, amino acid R37 is substituted by proline (P) and the amino acids R71 and S72 are deleted and amino acids R74, R77 and R104 are mutated to glutamine
(25) G1, P2, E3 are deleted, amino acid R36 and R37 are both substituted by glutamine (Q) and the amino acids R71 and S72 are deleted and amino acid R74 is mutated to glutamine (Q).
(26) G1, P2, E3 are deleted, amino acid R36 and R37 are both substituted by glutamine (Q) and the amino acids R71 and S72 are deleted and amino acids R74 and R77 are mutated to glutamine (Q).
(27) G1, P2, E3 are deleted, amino acid R36 is substituted by glutamine (Q), R37 is substituted by alanine and the amino acids R71 and S72 are deleted and amino acids R74 and R77 are mutated to glutamine (Q).
(28) G1, P2, E3 are deleted, amino acid R36 and R37 are both substituted by glutamine (Q) and the amino acids R71 and S72 are deleted and amino acids R74, R77 and R104 are mutated to glutamine (Q).
(29) G1, P2, E3 are deleted, amino acid R36 is substituted by glutamine (Q), R37 is substituted by alanine and the amino acids R71 and S72 are deleted and amino acids R74, R77 and R104 are mutated to glutamine (Q).
(30) G1, P2, E3 are deleted, amino acid R36 is substituted or deleted and the amino acids K68, S69, A70, R71 and S72 are deleted.
(31) G1, P2, E3 are deleted, amino acid R36 is substituted by glutamine (Q) and the amino acids K68, S69, A70, R71 and S72 are deleted.
(32) G1, P2, E3 are deleted, amino acid R37 is substituted or deleted and the amino acids K68, S69, A70, R71 and S72 are deleted.
(33) G1, P2, E3 are deleted, amino acid R37 is substituted by glutamic acid (E) and the amino acids K68, S69, A70, R71 and S72 are deleted.
(34) G1, P2, E3 are deleted, amino acid R37 is substituted by alanine and the amino acids K68, S69, A70, R71 and S72 are deleted.
(35) G1, P2, E3 are deleted, amino acid R37 is substituted by proline (P) and the amino acids K68, S69, A70, R71 and S72 are deleted.
(36) G1, P2, E3 are deleted, amino acids R36 and R37 are substituted or deleted and the amino acids K68, S69, A70, R71 and S72 are deleted.
(37) G1, P2, E3 are deleted, amino acid R36 and R37 are both substituted by glutamine (Q) and the amino acids K68, S69, A70, R71 and S72 are deleted.
(38) G1, P2, E3 are deleted, amino acid R36 is substituted by glutamine (Q), R37 is substituted by alanine and the amino acids K68, S69, A70, R71 and S72 are deleted.
(39) G1, P2, E3 are deleted, amino acid R36 is substituted or deleted and the amino acids K68, S69, A70, R71 and S72 are deleted and amino acid R74 is mutated to glutamine (Q).
(40) G1, P2, E3 are deleted, amino acid R36 is substituted or deleted and the amino acids K68, S69, A70, R71 and S72 are deleted and amino acids R74 and R77 are mutated to glutamine (Q).
(41) G1, P2, E3 are deleted, amino acid R36 is substituted or deleted and the amino acids K68, S69, A70, R71 and S72 are deleted and amino acids R74, R77 and Q104 are mutated to glutamine (Q).
(42) G1, P2, E3 are deleted, amino acid R36 is substituted by glutamine (Q) and the amino acids K68, S69, A70, R71 and S72 are deleted and amino acid R74 is mutated to glutamine (Q).
(43) G1, P2, E3 are deleted, amino acid R36 is substituted by glutamine (Q) and the amino acids K68, S69, A70, R71 and S72 are deleted and amino acids R74 and R77 are mutated to glutamine (Q).
(44) G1, P2, E3 are deleted, amino acid R36 is substituted by glutamine (Q) and the amino acids K68, S69, A70, R71 and S72 are deleted and amino acids R74, R77 and R104 are mutated to glutamine (Q).
(45) G1, P2, E3 are deleted, amino acid R37 is substituted by glutamic acid (E) and the amino acids K68, S69, A70, R71 and S72 are deleted and amino acid R74 is mutated to glutamine (Q).
(46) G1, P2, E3 are deleted, amino acid R37 is substituted by glutamic acid (E) and the amino acids K68, S69, A70, R71 and S72 are deleted and amino acids R74 and R77 are mutated to glutamine (Q).
(47) G1, P2, E3 are deleted, amino acid R37 is substituted by glutamic acid (E) and the amino acids K68, S69, A70, R71 and S72 are deleted and amino acids R74, R77 and R104 are mutated to glutamine (Q).
(48) G1, P2, E3 are deleted, amino acid R37 is substituted by alanine (A) and the amino acids K68, S69, A70, R71 and S72 are deleted and amino acid R74 is mutated to glutamine (Q).
(49) G1, P2, E3 are deleted, amino acid R37 is substituted by alanine (A) and the amino acids K68, S69, A70, R71 and S72 are deleted and amino acids R74 and R77 are mutated to glutamine (Q).
(50) G1, P2, E3 are deleted, amino acid R37 is substituted by alanine (A) and the amino acids K68, S69, A70, R71 and S72 are deleted and amino acids R74, R77 and R104 are mutated to glutamine
(51) G1, P2, E3 are deleted, amino acid R37 is substituted by proline (P) and the amino acids K68, S69, A70, R71 and S72 are deleted and amino acid R74 is mutated to glutamine (Q).
(52) G1, P2, E3 are deleted, amino acid R37 is substituted by proline (P) and the amino acids K68, S69, A70, R71 and S72 72 are deleted and amino acids R74 and R77 are mutated to glutamine (Q).
(53) G1, P2, E3 are deleted, amino acid R37 is substituted by proline (P) and the amino acids K68, S69, A70, R71 and S72 are deleted and amino acids R74, R77 and R104 are mutated to glutamine
(54) G1, P2, E3 are deleted, amino acid R36 and R37 are both substituted by glutamine (Q) and the amino acids K68, S69, A70, R71 and S72 are deleted and amino acid R74 is mutated to glutamine (Q).
(55) G1, P2, E3 are deleted, amino acid R36 and R37 are both substituted by glutamine (Q) and the amino acids K68, S69, A70, R71 and S72 are deleted and amino acids R74 and R77 are mutated to glutamine (Q).
(56) G1, P2, E3 are deleted, amino acid R36 is substituted by glutamine (Q), R37 is substituted by alanine and the amino acids K68, S69, A70, R71 and S72 are deleted and amino acids R74 and R77 are mutated to glutamine (Q).
(57) G1, P2, E3 are deleted, amino acid R36 and R37 are both substituted by glutamine (Q) and the amino acids K68, S69, A70, R71 and S72 are deleted and amino acids R74, R77 and R104 are mutated to glutamine (Q).
(58) G1, P2, E3 are deleted, amino acid R36 is substituted by glutamine (Q), R37 is substituted by alanine and the amino acids K68, S69, A70, R71 and S72 are deleted and amino acids R74, R77 and R104 are mutated to glutamine (Q).

In another embodiment the disclosure relates to the above described proteins comprising the polypeptides (1)-(58), wherein in said molecules, instead of being mutated at the positions 1-3, only the amino acid E3 is deleted.

Examples of such molecules include, but are not limited to, the following polypeptides:

A polypeptide comprising a human IGF-1Ea-peptide precursor protein wherein the amino acid(s)

(59) E3 is deleted, amino acid R36 is substituted or deleted and the amino acids R71 and S72 are deleted.
(60) E3 is deleted, amino acid R36 is substituted by glutamine (Q) and the amino acids R71 and S72 are deleted.
(61) E3 is deleted, amino acid R37 is substituted or deleted and the amino acids R71 and S72 are deleted.
(62) E3 is deleted, amino acid R37 is substituted by glutamic acid (E) and the amino acids R71 and S72 are deleted.
(63) E3 is deleted, amino acid R37 is substituted by alanine and the amino acids R71 and S72 are deleted.
(64) E3 is deleted, amino acid R37 is substituted by proline (P) and the amino acids R71 and S72 are deleted.
(65) E3 is deleted, amino acids R36 and R37 are substituted or deleted and the amino acids R71 and S72 are deleted.
(66) E3 is deleted, amino acid R36 and R37 are both substituted by glutamine (Q) and the amino acids R71 and S72 are deleted.
(67) E3 is deleted, amino acid R36 is substituted by glutamine (Q), R37 is substituted by alanine and the amino acids R71 and S72 are deleted.
(68) E3 is deleted, amino acid R36 is substituted or deleted and the amino acids R71 and S72 are deleted and amino acid R77 is mutated to glutamine (Q).
(69) E3 is deleted, amino acid R36 is substituted or deleted and the amino acids R71 and S72 are deleted and amino acids R74 and R77 are mutated to glutamine (Q).
(70) E3 is deleted, amino acid R36 is substituted or deleted and the amino acids R71 and S72 are deleted and amino acids R74, R77 and Q104 are mutated to glutamine (Q).
(71) E3 is deleted, amino acid R36 is substituted by glutamine (Q) and the amino acids R71 and S72 are deleted and amino acid R74 is mutated to glutamine (Q).
(72) E3 is deleted, amino acid R36 is substituted by glutamine (Q) and the amino acids R71 and S72 are deleted and amino acids R74 and R77 are mutated to glutamine (Q).
(73) E3 is deleted, amino acid R36 is substituted by glutamine (Q) and the amino acids R71 and S72 are deleted and amino acids R74, R77 and R104 are mutated to glutamine (Q).
(74) E3 is deleted, amino acid R37 is substituted by glutamic acid (E) and the amino acids R71 and S72 are deleted and amino acid R77 is mutated to glutamine (Q).
(75) E3 is deleted, amino acid R37 is substituted by glutamic acid (E) and the amino acids R71 and S72 are deleted and amino acids R74 and R77 are mutated to glutamine (Q).
(76) E3 is deleted, amino acid R37 is substituted by glutamic acid (E) and the amino acids R71 and S72 are deleted and amino acids R74, R77 and R104 are mutated to glutamine (Q).
(77) E3 is deleted, amino acid R37 is substituted by alanine (A) and the amino acids R71 and S72 are deleted and amino acid R74 is mutated to glutamine (Q).
(78) E3 is deleted, amino acid R37 is substituted by alanine (A) and the amino acids R71 and S72 are deleted and amino acids R74 and R77 are mutated to glutamine (Q).
(79) E3 is deleted, amino acid R37 is substituted by alanine (A) and the amino acids R71 and S72 are deleted and amino acids R74, R77 and R104 are mutated to glutamine
(80) E3 is deleted, amino acid R37 is substituted by proline (P) and the amino acids R71 and S72 are deleted and amino acid R74 is mutated to glutamine (Q).
(81) E3 is deleted, amino acid R37 is substituted by proline (P) and the amino acids R71 and S72 are deleted and amino acids R74 and R77 are mutated to glutamine (Q).
(82) E3 is deleted, amino acid R37 is substituted by proline (P) and the amino acids R71 and S72 are deleted and amino acids R74, R77 and R104 are mutated to glutamine
(83) E3 is deleted, amino acid R36 and R37 are both substituted by glutamine (Q) and the amino acids R71 and S72 are deleted and amino acid R74 is mutated to glutamine (Q).
(84) E3 is deleted, amino acid R36 and R37 are both substituted by glutamine (Q) and the amino acids R71 and S72 are deleted and amino acids R74 and R77 are mutated to glutamine (Q).
(85) E3 is deleted, amino acid R36 is substituted by glutamine (Q), R37 is substituted by alanine and the amino acids R71 and S72 are deleted and amino acids R74 and R77 are mutated to glutamine (Q).
(86) E3 is deleted, amino acid R36 and R37 are both substituted by glutamine (Q) and the amino acids R71 and S72 are deleted and amino acids R74, R77 and R104 are mutated to glutamine (Q).
(87) E3 is deleted, amino acid R36 is substituted by glutamine (Q), R37 is substituted by alanine and the amino acids R71 and S72 are deleted and amino acids R74, R77 and R104 are mutated to glutamine (Q).
(88) E3 is deleted, amino acid R36 is substituted or deleted and the amino acids K68, S69, A70, R71 and S72 are deleted.
(89) E3 is deleted, amino acid R36 is substituted by glutamine (Q) and the amino acids K68, S69, A70, R71 and S72 are deleted.
(90) E3 is deleted, amino acid R37 is substituted or deleted and the amino acids K68, S69, A70, R71 and S72 are deleted.
(91) E3 is deleted, amino acid R37 is substituted by glutamic acid (E) and the amino acids K68, S69, A70, R71 and S72 are deleted.
(92) E3 is deleted, amino acid R37 is substituted by alanine and the amino acids K68, S69, A70, R71 and S72 are deleted.
(93) E3 is deleted, amino acid R37 is substituted by proline (P) and the amino acids K68, S69, A70, R71 and S72 are deleted.
(94) E3 is deleted, amino acids R36 and R37 are substituted or deleted and the amino acids K68, S69, A70, R71 and S72 are deleted.
(95) E3 is deleted, amino acid R36 and R37 are both substituted by glutamine (Q) and the amino acids K68, S69, A70, R71 and S72 are deleted.
(96) E3 is deleted, amino acid R36 is substituted by glutamine (Q), R37 is substituted by alanine and the amino acids K68, S69, A70, R71 and S72 are deleted.
(97) E3 is deleted, amino acid R36 is substituted or deleted and the amino acids K68, S69, A70, R71 and S72 are deleted and amino acid R74 is mutated to glutamine (Q).
(98) E3 is deleted, amino acid R36 is substituted or deleted and the amino acids K68, S69, A70, R71 and S72 are deleted and amino acids R74 and R77 are mutated to glutamine (Q).
(99) E3 is deleted, amino acid R36 is substituted or deleted and the amino acids K68, S69, A70, R71 and S72 are deleted and amino acids R74, R77 and Q104 are mutated to glutamine (Q).
(100) E3 is deleted, amino acid R36 is substituted by glutamine (Q) and the amino acids K68, S69, A70, R71 and S72 are deleted and amino acid R74 is mutated to glutamine (Q).
(101) E3 is deleted, amino acid R36 is substituted by glutamine (Q) and the amino acids K68, S69, A70, R71 and S72 are deleted and amino acids R74 and R77 are mutated to glutamine (Q).
(102) E3 is deleted, amino acid R36 is substituted by glutamine (Q) and the amino acids K68, S69, A70, R71 and S72 are deleted and amino acids R74, R77 and R104 are mutated to glutamine (Q).
(103) E3 is deleted, amino acid R37 is substituted by glutamic acid (E) and the amino acids K68, S69, A70, R71 and S72 are deleted and amino acid R74 is mutated to glutamine (Q).
(104) E3 is deleted, amino acid R37 is substituted by glutamic acid (E) and the amino acids K68, S69, A70, R71 and S72 are deleted and amino acids R74 and R77 are mutated to glutamine (Q).
(105) E3 is deleted, amino acid R37 is substituted by glutamic acid (E) and the amino acids K68, S69, A70, R71 and S72 are deleted and amino acids R74, R77 and R104 are mutated to glutamine (Q).
(106) E3 is deleted, amino acid R37 is substituted by alanine (A) and the amino acids K68, S69, A70, R71 and S72 are deleted and amino acid R74 is mutated to glutamine (Q).
(107) E3 is deleted, amino acid R37 is substituted by alanine (A) and the amino acids K68, S69, A70, R71 and S72 are deleted and amino acids R74 and R77 are mutated to glutamine (Q).
(108) E3 is deleted, amino acid R37 is substituted by alanine (A) and the amino acids K68, S69, A70, R71 and S72 are deleted and amino acids R74, R77 and R104 are mutated to glutamine
(109) E3 is deleted, amino acid R37 is substituted by proline (P) and the amino acids K68, S69, A70, R71 and S72 are deleted and amino acid R74 is mutated to glutamine (Q).
(110) E3 is deleted, amino acid R37 is substituted by proline (P) and the amino acids K68, S69, A70, R71 and S72 are deleted and amino acids R74 and R77 are mutated to glutamine (Q).
(111) E3 is deleted, amino acid R37 is substituted by proline (P) and the amino acids K68, S69, A70, R71 and S72 are deleted and amino acids R74, R77 and R104 are mutated to glutamine
(112) E3 is deleted, amino acid R36 and R37 are both substituted by glutamine (Q) and the amino acids K68, S69, A70, R71 and S72 are deleted and amino acid R74 is mutated to glutamine (Q).
(113) E3 is deleted, amino acid R36 and R37 are both substituted by glutamine (Q) and the amino acids K68, S69, A70, R71 and S72 are deleted and amino acids R74 and R77 are mutated to glutamine (Q).
(114) E3 is deleted, amino acid R36 is substituted by glutamine (Q), R37 is substituted by alanine and the amino acids K68, S69, A70, R71 and S72 are deleted and amino acids R74 and R77 are mutated to glutamine (Q).
(115) E3 is deleted, amino acid R36 and R37 are both substituted by glutamine (Q) and the amino acids K68, S69, A70, R71 and S72 are deleted and amino acids R74, R77 and R104 are mutated to glutamine (Q).
(116) E3 is deleted, amino acid R36 is substituted by glutamine (Q), R37 is substituted by alanine and the amino acids K68, S69, A70, R71 and S72 are deleted and amino acids R74, R77 and R104 are mutated to glutamine (Q).

In another embodiment the disclosure relates to the above described compositions comprising the human IGF-1 precursor variants (e.g. the polypeptide variants 1-116) pegylated as described above, comprising a mutated E-peptide consisting of the amino acids

(SEQ ID. NO.: 99)
a) VQAQQHTDMPKTQKEVHLKNASG,
or
(SEQ ID. NO.: 100)
b) VQAQQHTDMPKTQKYQPPATNKNTKSQRRKGS
(SEQ ID.NO.: 101)
c) VQAQQHTDMPKTQKEVHLKNASRGSAGNKNYQM.

In one embodiment, the invention provides the above described compositions comprising the mono pegylated human IGF-1 precursor proteins, wherein the human IGF-1Ea precursor polypeptide comprises the mutations described in human IGF-1Ea precursor polypeptide variant 63 above.

In one embodiment, the present disclosure provides the above described compositions comprising the mono pegylated human IGF-1 precursor proteins, wherein the pegylated human IGF-1 precursor protein is at least 95%, at least 96%, at least 96%, at least 97%, at least 98%, at least 99% identical to SEQ ID NO: 55.

In one embodiment, the invention provides the above described composition comprising the mono pegylated human IGF-1 precursor proteins, wherein the human IGF-1Ea precursor polypeptide comprises the amino acid sequence of SEQ ID NO: 55.

Consequently, one embodiment of the invention also relates to the above described compositions of pegylated human IGF-1 precursor proteins, directly obtained from a pegylation process, wherein in said composition at least 64 percent of the mono pegylated fraction of said composition is N-terminally mono-pegylated IGF-1 precursor protein and wherein the human IGF-1Ea precursor polypeptide consists of the amino acid sequence of SEQ ID NO: 55.

The above described composition of pegylated human IGF-1 precursor proteins, wherein in said composition at least 61 percent, or at least 62 percent, or at least 63 percent, or at least 64 percent, or at least 65, or at least 66, or at least 67 percent of the mono pegylated IGF-1 precursor protein fraction of said composition is N-terminally mono-pegylated IGF-1 precursor protein can be produced by a process comprising the steps of reacting in an aqueous medium said IGF-1 precursor proteins with a water-soluble polyethylene glycol, e.g. a linear PEG, under conditions of a reductive alkylation, characterized in that the coupling reaction is performed within a pH range of about 6.5 to 7.5.

In an additional embodiment the disclosure relates to a composition of pegylated human IGF-1 precursor proteins, directly resulting from the pegylation process described herein, wherein in said composition at least 61 percent, or at least 62 percent, or at least 63 percent, or at least 64 percent, or at least 65, or at least 66, or at least 67 percent of the mono pegylated IGf-1 precursor protein fraction of said composition is N-terminally mono-pegylated IGF-1 precursor protein, obtained by a process comprising the step of reacting human IGF-1Ea peptide precursor proteins in an aqueous medium with a water-soluble polyethylene glycol under conditions of a reductive alkylation, characterized in that the coupling reaction is performed within a pH range of about 6.5 to 7.5.

In a particular embodiment, the above described composition is obtained by a process of reacting human IGF-1Ea peptide precursor proteins in an aqueous medium with a water-soluble polyethylene glycol under conditions of a reductive alkylation, characterized in that the coupling reaction is performed within a pH range of about 6.5 to 7.5 in the presence of α-cyclodextrine (α-CD).

Hence, in another particular embodiment the disclosure relates to above described composition of pegylated human IGF-1 precursor proteins directly resulting from the pegylation process described herein, wherein in said composition at least 61 percent, or at least 62 percent, or at least 63 percent, or at least 64 percent, or at least 65, or at least 66, or at least 67 percent of the mono-pegylated IGF-1 precursor protein fraction of said composition is N-terminally mono-pegylated IGF-1 precursor protein obtainable from reacting human IGF-1Ea peptide precursor proteins in an aqueous medium with a water-soluble polyethylene glycol under conditions of a reductive alkylation, characterized in that the coupling reaction is performed within a pH range of about 6.5 to 7.5. In an additional embodiment of the disclosure the above described reductive alkylation reactions are performed at a pH of about 6.5

In a particular embodiment, the above described composition is obtainable from reacting human IGF-1Ea peptide precursor proteins in an aqueous medium with a water-soluble polyethylene glycol under conditions of a reductive alkylation, characterized in that the coupling reaction is performed at a pH of about 6.5 in the presence of α-cyclodextrine (α-CD).

Another embodiment of the disclosure relates to a process for preparing a composition of pegylated human IGF-1 precursor proteins directly resulting from a pegylation process, wherein in said composition at least 61 percent, or at least 62 percent, or at least 63 percent, or at least 64 percent, or at least 65, or at least 66, or at least 67 percent of the mono pegylated fraction of said composition is N-terminally mono-pegylated IGF-1 precursor protein, comprising the steps of reacting in an aqueous medium an IGF-1 precursor protein with a water-soluble polyethylene glycol under conditions of a reductive alkylation, characterized in that the coupling reaction is performed at a pH of about 6.5.

In a particular embodiment the disclosure relates to a process for preparing a composition of pegylated human IGF-1 precursor proteins, directly resulting from a pegylation process, wherein in said composition at least 61 percent, or at least 62 percent, or at least 63 percent, or at least 64 percent, or at least 65, or at least 66, or at least 67 percent of the mono pegylated fraction of said composition is N-terminally mono-pegylated IGF-1 precursor protein, comprising the steps of reacting in an aqueous medium an IGF-1 precursor protein with a water-soluble polyethylene glycol under conditions of a reductive alkylation, characterized in that the coupling reaction is performed within a pH range of about 6.5 to 7.5 in the presence of α-cyclodextrine (α-CD).

The α-cyclodextrine (α-CD) can be used in a final concentration ranging from 1 to 5% (1 to 5 g/100 ml). In a particular embodiment, solid α-CD was added to the reaction mixture up to 3% final concentration

In a particular embodiment of the disclosure, the PEG used in the above described processes is linear and has an overall molecular weight of from 20 to 100 kDa, or from 20 to 80 kDa, or from 20 to 70 kDa, or from 20 to 60 kDa, or from 20 to 50 kDa. Accordingly, in one embodiment of the invention, the PEG used in the above described process is linear PEG and has an overall molecular weight of about 30 kDa. The PEG used as described above can be linear or branched.

The above described process can comprise additional steps allowing for further purification, separation, enrichment of pegylated proteins or desired protein fractions of those proteins directly resulting from the above described pegylation step. The skilled person is aware of feasible technologies (Fee et al, Chemical Engineering Science Chemical Engineering Science, 61 (2006) 924-939), Pabst et al, Journal of Chromatography A, 1147 (2007) 172-182, Lee et al, Pharmaceutical Research, 20 2003 818-85). In a particular embodiment these additional step can be a cation exchange chromatography (CEX) (Yun et al, Journal of Biotechnology, 118 (2005) 67-74, Jevsevar et al, Biotechnol. J., 5 (2010) 113-128, Molineux, Current Pharmaceutical Design, 10 (2004) 1235-1244), Baker et al, Bioconjugate Chem. 17 (2006) 179-188). The present invention also describes the purification/isolation process which employs cation exchange chromatography (CEX) in a way where a partial separation between lysine (Lys) and N-terminally PEGylated isoforms is achieved and enables control over the composition of PEGylated isoforms in final mono-PEGylated product.

Surprisingly, under the above described inventive process conditions, the addition of α-CD to the PEGylation mixture reduces formation of higher PEGylated forms and also slows down the PEGylation reaction. Addition of α-CD to the PEGylation mixture reduces the amount of higher PEGylated forms, simplifies chromatographic purification and thereby assures higher yield because of simplified chromatographic purification. α-CD added to the pegylation process improves lot to lot consistency and ensure high yields and high reproducibility of the process. Lot to lot consistency is also controlled by a cation exchange chromatography purification step which is performed in a way where partial separation of lysine-PEGylated and N-terminally PEGylated isoforms is achieved.

Another embodiment of the disclosure relates to a process for preparing a composition of mono pegylated human IGF-1 precursor proteins, wherein said composition comprises at least 70 percent, or at least 80 percent, or at least 90 percent, or at least 91 percent, or at least 92 percent, or at least 93 percent, or at least 94 percent, or at least 95 percent, or at least 96 percent, or at least 97 percent, or more of mono-pegylated IGF-1 precursor protein, comprising the steps of

(a) reacting in an aqueous medium an IGF-1 precursor protein with a water-soluble polyethylene glycol under conditions of a reductive alkylation, characterized in that the coupling reaction is performed at a pH of about 6.5 in the presence of α-cyclodextrine (α-CD),
(b) performing an exchange chromatography step with the compositions obtained in step (a),
(c) obtaining elution fractions of the exchange chromatography step (b), and
(d) pool those fractions containing the mono-pegylated IGF-1Ea precursor protein isoforms.

In a particular embodiment of the disclosure, the above described step (b) is a cation exchange chromatography (CEX).

Likewise, in addition to the above described exchange chromatography step (b), e.g. CEX, the following concentration/purification steps can be added to the above disclosed process:

(e) UF/DF concentration and buffer exchange

The ultrafiltration and diafiltration step is performed with aim to concentrate and to exchange the CEX chromatography buffer into the final buffer (Example 4; UF/DF step, sterile filtration and filling).

(f) final filtration and filling (Example 4)

Consequently, another embodiment of the invention relates to compositions comprising more than 90 percent of mono-pegylated IGF-1 precursor proteins obtained by performing the above described process steps (a) to (d) and (a) to (f), respectively.

In a particular embodiment of the disclosure, the above disclosed process comprising the steps (a) to (d) and (a) to (f), respectively, is used for preparing a composition of variant 63 mono pegylated human IGF-1Ea precursor proteins (as described above)

Consequently, one embodiment of the disclosure refers to the above disclosed process comprising the steps (a) to (d) and (a) to (f), respectively, wherein said process is used for preparing a composition of mono pegylated human IGF-1Ea precursor proteins consisting of protein sequence of SEQ ID NO: 55.

The above described compositions comprising N-terminally and lysine residue mono-pegylated IGF-1 precursor proteins produced according to the above disclosed pegylation method/process can be further processed into pharmaceutically acceptable forms. The skilled person is aware of relevant formulation techniques available in the prior art.

Hence, the disclosure relates to the production of a pharmaceutical composition comprising an N-terminally or lysine residue mono-pegylated human IGF-1Ea precursor polypeptide of SEQ ID NO: 55, comprising the steps of

(a) reacting in an aqueous medium human IGF-1Ea precursor polypeptide of SEQ ID NO: 55 with a water-soluble polyethylene glycol under conditions of a reductive alkylation, characterized in that the coupling reaction is performed within a pH range of about 6.5 to 7.5 in the presence of α-cyclodextrine (α-CD), and
(b) performing an exchange chromatography step with the compositions obtained in step (a),
(c) obtaining elution fractions of the exchange chromatography step (b), and
(d) pooling those fractions containing the mono-pegylated IGF-1Ea precursor protein isoforms, and
(e) using the fraction obtained in step (d) for the preparation of a pharmaceutical composition.

### Chromatographic Purification

Mono-PEGylated IGF-1Ea precursor proteins are purified from excess reagents, reaction by-products and non-PEGylated IGF-1Ea precursor proteins by chromatography on strong cation exchange resin. The purification is performed at a pH which still enables retention of mono-PEGylated IGF-1Ea on the column and is at the same time high enough to achieve at least partial separation between lysine and N-terminally mono-PEGylated IGF-1Ea precursor proteins. A pH range between 6.5 and 7.5 was determined to be the most appropriate for the separation. Consequently, the disclosure relates to the above described processes, in which the chromatographic purification step (e.g. step (b) above) is performed at a pH of about 6.5. Alternatively, additional steps like UF/DF concentration, buffer exchange, and filtration can be applied in the above described process prior to the preparation of the pharmaceutical composition of step (e).

The resulting pharmaceutical composition comprising the mono-pegylated human IGF-1 precursor proteins produced according to the disclosed processes can be used in therapy. Hence, in one embodiment the disclosure relates to above mentioned specific and novel compositions comprising mono-pegylated human IGF-1 precursor proteins in a pharmaceutical acceptable form for use in therapy, particularly for therapeutic use in the treatment of a muscle disorder in a patient in need thereof. In a particular embodiment of the disclosure, the therapeutic use is the treatment of burn patients suffering from loss of lean body mass and/or muscle wasting or the treatment of chronic obstructive pulmonary disease (COPD) patients, or the treatment of Spinal and Bulbar Muscular Atrophy (SBMA or Kennedy disease) patients, or the treatment of chronic kidney disease patients.

In a particularly preferred embodiment the disclosure relates to the above specified pharmaceutical uses in therapy of those pharmaceutical compositions, which have been prepared by using compositions comprising at least 95 percent, or more of a mixture of N-terminally and lysine residue mono pegylated human IGF-1Ea peptide precursor protein consisting of SEQ ID NO: 55 prepared according to the above described processes.

The disclosure furthermore provides for a method of treating a muscle disorder in a patient in need thereof, the method comprising administering a therapeutically effective amount of N-terminally residue pegylated human IGF-1Ea peptide precursor protein comprising compositions produced according to the herein disclosed process.

Accordingly, in one particular embodiment, the disclosure relates to a method of treating burn patients suffering from loss of lean body mass and/or muscle wasting, or a method of treating chronic obstructive pulmonary disease (COPD) patients, or a method of treating Kennedy disease patients, or a method of treating chronic kidney disease patients, comprising administering a therapeutically effective amount of N-terminally residue pegylated human IGF-1Ea peptide precursor protein comprising compositions produced according to the herein disclosed process.

### Amino Acid Mutations

As referred to above, various amino acids may be mutated to another amino acid. However, other amino acids may be used, such as non-natural amino acids or natural amino acids from another group (i.e. polar, acidic, basic or non-polar). Methods of introducing a mutation into amino acids of a protein are well known to those skilled in the art. See, e.g., Ausubel (ed.), Current Protocols in Molecular Biology, John Wiley and Sons, Inc. (1994); T. Maniatis, E. F. Fritsch and J. Sambrook, Molecular Cloning: A Laboratory Manual, Cold Spring Harbor laboratory, Cold Spring Harbor, N.Y. (1989)). Methods comprise, but are not limited to, amplification of the DNA encoding the polypeptide functionally active variant or fragments thereof by polymerase chain reaction (PCR) conducted with mutagenic primers and assembling the fragments by assembly PCR if needed or introduction of mutations using commercially available kits such as “QuikChange™ Site-Directed Mutagenesis Kit” (Stratagene). See, e.g., Ausubel (ed.), Current Protocols in Molecular Biology, John Wiley and Sons, Inc. (1994); T. Maniatis, E. F. Fritsch and J. Sambrook, Molecular Cloning: A Laboratory Manual, Cold Spring Harbor laboratory, Cold Spring Harbor, N.Y. (1989)). Further, mutated sequences can be obtained by synthetic gene synthesis a service provided by commercial companies (e.g. Geneart, Life Technology). The generation of a polypeptide functionally active variant or derivative to a polypeptide by replacing an amino acid that does not influence the function of a polypeptide can be accomplished by one skilled in the art.

### Production of Therapeutic Proteins

Currently used state of the art heterologous protein production systems include prokaryotic and eukaryotic cell systems like E. coli, yeast, viruses, fungi and insect cells. In order to produce recombinant proteins which require post- or peri-translational modifications such as glycosylation (and where industrial scale production is needed) very often mammalian cell systems including cells from Cricetulus griseus, Cercopithecus aethiops, Homo sapiens, Mesocricetus auratus, Mus musculus and Chlorocebus species are used. Mammalian cell systems have become a routine production system for therapeutic proteins and antibodies. Said cells have been characterized extensively in the recent history, they can reach extremely high production levels, can be free of infectious or virus like particles, can grow to very high density in bioreactors and they can be genetically manipulated and transformed. For example, Chinese Hamster Ovary (CHO) cells can be engineered to resemble the human glycan profile by transfection of the appropriate glycosyl transferases. Recombinantly produced growth factors are already widely used in therapeutic applications or are promising candidates for the development of new therapies.

The skilled person knows how to transform, select and cultivate genetically modified mammalian cells, e.g. CHO cells, like CHO-K1 derivative, CHO-DUXB11 derivative or CHO-DG44 cells. Selection protocols are routinely used to facilitate selection of cells that are likely to have integrated the recombinant DNA encoding the desired therapeutic protein, like growth factors, e.g. IGF-1. Antibiotic resistance or the ability to grow in a nutritionally selective medium conferred by a gene co-integrated on the transformation vector is routinely used. (see Weber, W. and Fussenegger, M. (2003) Inducible gene expression in mammalian cells, in Gene transfer and expression in mammalian cells, (Makrides, S. C., Ed.), Elsevier: Amsterdam, pp. 589-604.) (Efficient selection for high-expression transfectants with a novel eukaryotic vector: Niwa Hitoshi, Yamamura Ken-ichi, Miyazaki Jun-ichi). The two most common CHO expression systems for recombinant protein production utilize dihydrofolate reductase (DHFR)-based methotrexate (MTX) selection or glutamine synthetase (GS)-based methionine sulfoximine (MSX) selection (Rita Costa A, Elisa Rodrigues M, Henriques M, Azeredo J, Oliveira R. Eur J Pharm Biopharm. 2010 February; 74(2):127-38. Epub 2009 Oct. 22. Guidelines to cell engineering for monoclonal antibody production).

Large scale production of polypeptides with transfected mammalian cells, e.g. CHO cells can be done for example in wave, glass or stainless steel bioreactors. For that purpose the cells are expanded, usually starting from a single frozen vial, for example a vial from a Master Cell Bank. The cells are thawed and expanded through several steps. Bioreactors of different scale are inoculated with appropriate amounts of cells. The cell density can be increased by adding feed solutions and additives to the bioreactor. Cells are kept at a high viability for a prolonged time. Product concentrations in the reactor ranging from a few hundred milligrams per liter up to several grams per liter are achieved in the large scale. Purification can be done by standard chromatography methodology, which can include affinity, ion exchange, hydrophobic interaction or size exclusion chromatography steps. The size of the bioreactor can be up to several thousand liters volume in the final scale (see also e.g. F. Wurm, Nature Biotechnology Vol. 22, 11, 2004, 1393-1398).

Therapeutic proteins can be produced very efficiently in E. coli using the Npro Autoprotease Fusion Technology (NAFT). The skilled person is aware of this technology which, inter alia, has been disclosed in great detail in patent applications/patents WO200111056, WO200111057, WO2006113957, EP1200604B1 and U.S. Pat. No. 6,936,455B1.

### Pharmaceutical Compositions

In another aspect, the present invention provides a composition, e.g. a pharmaceutical composition, containing one or a combination of the above described pegylated human IGF-1Ea peptide precursor protein compositions produced according to the method or process of the invention, formulated together with a pharmaceutically acceptable carrier. Pharmaceutical compositions of the invention also can be administered in combination therapy, i.e. combined with other agents. For example, pegylated human IGF-1Ea peptide precursor protein compositions produced according to the method or process of the invention can be combined with at least one muscle mass/strength increasing agent, for example, anti-ActRIIB antibody, anti-ActRIIA/B pan antibody IGF-2 or variants IGF-2, an anti-myostatin antibody, a myostatin propeptide, a myostatin decoy protein that binds ActRIIB but does not activate it, a beta 2 agonist, a Ghrelin agonist, a SARM, GH agonists/mimetics or follistatin. Examples of therapeutic agents that can be used in combination therapy are described in greater detail below in the section on uses of the IGF-1 variants of the invention.

The term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin.

In a preferred embodiment, the composition is formulated in accordance with routine procedures as a pharmaceutical composition adapted for intravenous administration to human beings. Where necessary, the composition may also include a solubilizing agent and a local anesthetic such as lidocaine to ease pain at the site of the injection. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

Pharmaceutically acceptable carrier include any and all solvents, dispersion media, coatings, antibacterial and antifungal agents, isotonic and absorption delaying agents, and the like that are physiologically compatible. The carrier should be suitable for intravenous, intramuscular, subcutaneous, parenteral, spinal or epidermal administration (e.g. by injection or infusion). Depending on the route of administration, the active compound, i.e. antibody, growth factors, immunoconjuage, or a bispecific molecule, may be coated in a material to protect the compound from the action of acids and other natural conditions that may inactivate the compound.

A pharmaceutical composition of the invention also may include a pharmaceutically acceptable anti-oxidant. Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol, and the like; and metal chelating agents, such as citric acid, ethylenediamine tetraacetic acid (EDTA), sorbitol, tartaric acid, phosphoric acid, and the like. Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as, aluminum monostearate and gelatin.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the invention is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, one can include isotonic agents, for example, sugars, polyalcohols such as mannitol, sorbitol, or sodium chloride in the composition. Prolonged absorption of the injectable compositions can be brought about by including in the composition an agent that delays absorption for example, monostearate salts and gelatin.

Sterile injectable solutions can be prepared by incorporating the active compound in the required amount in an appropriate solvent with one or a combination of agents enumerated above, as required, followed by sterilization microfiltration. Generally, dispersions are prepared by incorporating the active compound into a sterile vehicle that contains a basic dispersion medium and the required other agents from those enumerated above. In the case of sterile powders for the preparation of sterile injectable solutions, the methods of preparation are vacuum drying and freeze-drying (lyophilization) that yield a powder of the active agent plus any additional desired agent from a previously sterile-filtered solution thereof.

The amount of active agent which can be combined with a carrier material to produce a single dosage form will vary depending upon the subject being treated, and the particular mode of administration. The amount of active agent which can be combined with a carrier material to produce a single dosage form will generally be that amount of the composition which produces a therapeutic effect. Generally, out of one hundred percent, this amount will range from about 0.01 percent to about ninety-nine percent of active agent, from about 0.1 percent to about 70 percent, or from about 1 percent to about 30 percent of active agent in combination with a pharmaceutically acceptable carrier.

Dosage regimens are adjusted to provide the optimum desired response (e.g. a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of sensitivity in individuals.

A therapeutically effective amount of a polypeptide in the context of administrating the pegylated human IGF-1Ea peptide precursor protein compositions produced according to the method or process of the invention, ranges from about 0.001 to 10 mg/kg and more usually 0.1 to 10 mg/kg of the host body weight. For example dosages can be about 0.1 mg/kg body weight, can be about 0.2 mg/kg body weight, can be about 0.3 mg/kg body weight, can be about 1 mg/kg body weight, can be about 3 mg/kg body weight, can be about 5 mg/kg body weight or about 10 mg/kg body weight. The skilled person knows to identify a suitable effective dose, which will vary depending on the rout of administration (e.g. intravenously or subcutaneously). An exemplary treatment regime entails administration once per day, once every week, once every two weeks, once every three weeks, once every four weeks or once a month. Such administration may be carried out intravenously or subcutaneously. Dosage regimens for pegylated human IGF-1Ea peptide precursor protein compositions produced according to the method or process of the invention include 0.1 mg/kg body weight or 0.2 mg/kg body weight or 0.3 mg/kg body weight or 0.5 mg/kg body weight or 1 mg/kg body weight or 3 mg/kg body weight or 10 mg/kg body weight by intravenous administration. Alternatively, the composition can be a sustained release formulation, in which case less frequent administration is required. Dosage and frequency vary depending on the half-life of the antibody in the patient. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated or until the patient shows partial or complete amelioration of symptoms of disease. Thereafter, the patient can be administered a prophylactic regime.

Actual dosage levels of the active agents in the pharmaceutical compositions of the present invention may be varied so as to obtain an amount of the active agent which is effective to achieve the desired therapeutic response for a particular patient, composition, and mode of administration, without being toxic to the patient. The selected dosage level will depend upon a variety of pharmacokinetic factors including the activity of the particular compositions of the present invention employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

Administration of a therapeutically effective dose of an pegylated human IGF-1Ea peptide precursor protein compositions produced according to the method or process of the invention can result in a decrease in severity of disease symptoms, an increase in frequency and duration of disease symptom-free periods, or a prevention of impairment or disability due to the disease affliction i.e. an increase in muscle mass and/or function or decrease/reduction of wound area in burn patients.

Patients will receive an effective amount of the polypeptide active ingredient i.e. an amount that is sufficient to detect, treat, ameliorate, or prevent the disease or disorder in question. Therapeutic effects may also include reduction in physical symptoms. The optimum effective amount and concentration of a therapeutic protein for any particular subject will depend upon various factors, including the patient's age size health and/or gender, the nature and extent of the condition, the activity of the particular therapeutic protein, the rate of its clearance by the body, and also on any possible further therapeutic(s) administered in combination with the therapeutic protein. The effective amount delivered for a given situation can be determined by routine experimentation and is within the judgment of a clinician. Dosage can be by a single dose schedule or a multiple dose schedule.

A composition of the present invention can be administered by one or more routes of administration using one or more of a variety of methods known in the art. As will be appreciated by the skilled artisan, the route and/or mode of administration will vary depending upon the desired results. Routes of administration for the therapeutic proteins of the invention include intravenous, intramuscular, intradermal, intraperitoneal, subcutaneous, spinal or other parenteral routes of administration, for example by injection or infusion. The phrase “parenteral administration” as used herein means modes of administration other than enteral and topical administration, usually by injection, and includes, without limitation, intravenous, intramuscular, intra-arterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrastemal injection and infusion. In one embodiment the antibody comprising composition is administered intravenously. In another embodiment the antibody is administered subcutaneously.

Alternatively, a pegylated human IGF-1Ea peptide precursor protein composition produced according to the method or process of the invention can be administered by a nonparenteral route, such as a topical, epidermal or mucosal route of administration, for example, intranasally, orally, vaginally, rectally, sublingually or topically.

The active compounds can be prepared with carriers that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g. Sustained and Controlled Release Drug Delivery Systems, J. R. Robinson, ed., Marcel Dekker, Inc., New York, 1978.

Therapeutic compositions can be administered with medical devices known in the art. For example, in one embodiment, a therapeutic composition of the invention can be administered with a needleless hypodermic injection device, such as the devices shown in U.S. Pat. No. 5,399,163; 5,383,851; 5,312,335; 5,064,413; 4,941,880; 4,790,824 or 4,596,556. Examples of well-known implants and modules useful in the present invention include: U.S. Pat. No. 4,487,603, which shows an implantable micro-infusion pump for dispensing medication at a controlled rate; U.S. Pat. No. 4,486,194, which shows a therapeutic device for administering medicants through the skin; U.S. Pat. No. 4,447,233, which shows a medication infusion pump for delivering medication at a precise infusion rate; U.S. Pat. No. 4,447,224, which shows a variable flow implantable infusion apparatus for continuous drug delivery; U.S. Pat. No. 4,439,196, which shows an osmotic drug delivery system having multi-chamber compartments; and U.S. Pat. No. 4,475,196, which shows an osmotic drug delivery system. Many other such implants, delivery systems, and modules are known to those skilled in the art and include those made by MicroCHIPS™ (Bedford, Mass.).

In certain embodiments, the pegylated human IGF-1Ea peptide precursor protein compositions produced according to the method or process of the invention can be formulated to ensure proper distribution in vivo. For example, the blood-brain barrier (BBB) excludes many highly hydrophilic compounds. To ensure that the therapeutic compounds of the invention cross the BBB (if desired); they can be formulated, for example, in liposomes. For methods of manufacturing liposomes, see, e.g. U.S. Pat. Nos. 4,522,811; 5,374,548; and 5,399,331. The liposomes may comprise one or more moieties which are selectively transported into specific cells or organs, thus enhance targeted drug delivery (see, e.g. V. V. Ranade, 1989 J. Clin Pharmacol. 29:685). Exemplary targeting moieties include folate or biotin (see, e.g. U.S. Pat. No. 5,416,016); mannosides (Umezawa et al., 1988 Biochem. Biophys. Res. Commun. 153:1038); antibodies (P. G. Bloeman et al., 1995 FEBS Lett. 357:140; M. Owais et al., 1995 Antimicrob. Agents Chernother. 39:180); surfactant protein A receptor (Briscoe et al., 1995 Am. J. Physiol. 1233:134); p 120 (Schreier et al., 1994 J. Biol. Chem. 269:9090); see also K. Keinanen; M. L. Laukkanen, 1994 FEBS Lett. 346:123; J. J. Killion; I. J. Fidler, 1994 Immunomethods 4:273.

### Target Diseases and Disorders

The invention provides a composition of pegylated human IGF-1Ea peptide precursor proteins produced according to the method or process of the invention or useful pharmaceutical composition thereof for use in therapy. The invention further provides a pharmaceutical composition of pegylated human IGF-1Ea peptide precursor protein compositions produced according to the method or process of the invention for use in the treatment of a pathological disorder. The invention further provides use of a composition of pegylated human IGF-1Ea peptide precursor proteins produced according to the method or process of the invention in the manufacture of a medicament for the treatment of a pathological disorder. The invention further provides a method of treating a patient suffering from a pathological disorder comprising administering a therapeutically effective amount of pegylated human IGF-1Ea peptide precursor proteins produced according to the method or process of the invention to said patient.

The pathological disorder may be a musculoskeletal disease or disorder, such as muscle atrophy. There are many causes of muscle atrophy, including as a result of treatment with a glucocorticoid such as cortisol, dexamethasone, betamethasone, prednisone, methylprednisolone, or prednisolone. The muscle atrophy can also be a result of denervation due to nerve trauma or a result of degenerative, metabolic, or inflammatory neuropathy (e.g., Guillian-Barré syndrome, peripheral neuropathy, or exposure to environmental toxins or drugs).

In addition, the muscle atrophy can be a result of myopathy, such as myotonia; a congenital myopathy, including nemalene myopathy, multi/minicore myopathy and myotubular (centronuclear) myopathy; mitochondrial myopathy; familial periodic paralysis; inflammatory myopathy; metabolic myopathy, such as caused by a glycogen or lipid storage disease; dermatomyositisis; polymyositis; inclusion body myositis; myositis ossificans; rhabdomyolysis and myoglobinurias.

In another embodiment of the disclosure, the pharmaceutical composition of the invention can be used for the treatment of Kennedy Disease or chronic kidney disease

The myopathy may be caused by a muscular dystrophy syndrome, such as Duchenne, Becker, myotonic, fascioscapulohumeral, Emery-Dreifuss, oculopharyngeal, scapulohumeral, limb girdle, Fukuyama, a congenital muscular dystrophy, or hereditary distal myopathy. The musculoskeletal disease can also be osteoporosis, a bone fracture, short stature, or dwarfism.

In addition, the muscle atrophy can be a result of an adult motor neuron disease such as amyotrophic lateral sclerosis; infantile spinal muscular atrophy, juvenile spinal muscular atrophy, autoimmune motor neuropathy with multifocal conductor block, paralysis due to stroke or spinal cord injury, skeletal immobilization due to trauma, prolonged bed rest, voluntary inactivity, involuntary inactivity, metabolic stress or nutritional insufficiency, cancer, AIDS, fasting, a thyroid gland or adrenal gland or pituitary gland disorder, diabetes, benign congenital hypotonia, central core disease, liver diseases (examples such as fibrosis, cirrhosis), sepsis, renal failure, congestive heart failure, ageing, space travel or time spent in a zero gravity environment.

In a particular embodiment, the pharmaceutical composition of the invention can be used for the treatment of burn patients including adult and pediatric burn injury, suffering from loss of lean body mass and/or muscle wasting.

Examples of age-related conditions that may be treated include sarcopenia, skin atrophy, muscle wasting, brain atrophy, atherosclerosis, arteriosclerosis, pulmonary emphysema, osteoporosis, osteoarthritis, immunologic incompetence, high blood pressure, dementia, Huntington's disease, Alzheimer's disease, cataracts, age-related macular degeneration, prostate cancer, stroke, diminished life expectancy, frailty, memory loss, wrinkles, impaired kidney function, and age-related hearing loss; metabolic disorders, including Type II Diabetes, Metabolic Syndrome, hyperglycemia, and obesity.

In a particular embodiment, the pharmaceutical composition of the invention can be used for the treatment of chronic obstructive pulmonary disease (COPD) patients

In another embodiment of the disclosure the pharmaceutical composition of the invention can be used for the treatment of muscle atrophy. In a particular embodiment the disclosure relates to the use of the pharmaceutical composition of the invention for the treatment of muscle atrophy, wherein the atrophy group is selected from the group consisting of obesity-associated sarcopenia, sarcopenia, and diabetes-associated muscle atrophy.

Other conditions that may be treated include acute and/or chronic renal disease or failure, liver fibrosis or cirrhosis, cancer such as pancreatic, gastrointestinal (including esophageal, gastric, and colon), lung, prostate, lymphoma, or breast cancer; Parkinson's Disease; conditions associated with neuronal death, such as ALS (amyotrophic lateral sclerosis), brain atrophy, or dementia and anemia; chronic infections such as tuberculosis, whether caused by Mycobacterium tuberculosis or by atypical mycobacteria; chronic fungal infections; and opportunistic infections in the setting of immune suppression, whether iatrogenic or due to AIDS.

Further conditions include cachexia, cachexia associated with a rheumatoid arthritis and cachexia associated with cancer.

In another embodiment the disclosure relates to method of treating a muscle disorder, the method comprising administering a therapeutically effective amount of pegylated human IGF-1Ea peptide precursor protein compositions produced according to the method or process of the invention, as described above. The need of treatment with the disclosed polypeptides or compositions to increase muscle mass can result from one of the above mentioned conditions, particularly as a consequence of a musculoskeletal disease or disorder, such as muscle atrophy, wherein the muscle disorder is a muscle atrophy selected from the group consisting of obesity-associated sarcopenia, sarcopenia, and diabetes-associated muscle atrophy.

Additionally, the disclosure relates to method of treating a burn injury, a chronic obstructive pulmonary disease (COPD), an age related condition like sarcopenia, the Kennedy disease, or a chronic kidney disease, comprising administering a therapeutically effective amount to a patient, as described above, of pegylated human IGF-1Ea peptide precursor protein compositions produced according to the method or process of the invention.

In another embodiment, the disclosure relates to a method for increasing muscle mass. In a particular embodiment, the disclosure relates to a method for increasing muscle mass in a patient in need thereof. The need to increase muscle mass can result from one of the above mentioned conditions, particularly as a consequence of a musculoskeletal disease or disorder, such as muscle atrophy. The need to increase muscle mass can also result from a burn injury, a chronic obstructive pulmonary disease (COPD), an age related condition like sarcopenia, the Kennedy disease, or a chronic kidney disease.

### Patient Administration

A pharmaceutical composition of the invention can be administered to a patient. Administration will typically be via a syringe. Thus the invention provides a delivery device (e.g. a syringe) including a pharmaceutical composition of the invention.

Various delivery systems are known and can be used to administer the polypeptide of the invention, e.g., encapsulation in liposomes, microparticles, microcapsules, recombinant cells capable of expressing the protein, receptor-mediated endocytosis (see, e.g., Wu and Wu, J Biol Chem 262:4429-4432, 1987), construction of a nucleic acid as part of a retroviral, adeno-associated viral, adenoviral, poxviral (e.g., avipoxviral, particularly fowlpoxviral) or other vector, etc. Methods of introduction can be enteral or parenteral and include but are not limited to intradermal, intramuscular, intraperitoneal, intravenous, subcutaneous, pulmonary, intranasal, intraocular, epidural, and oral routes. The polypeptides can be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compositions of the invention into the central nervous system by any suitable route, including intraventricular and intrathecal injection; intraventricular injection may be facilitated by an intraventricular catheter, for example, attached to a reservoir, such as an Ommaya reservoir. In a specific embodiment, it may be desirable to administer the pharmaceutical compositions of the invention locally to the area in need of treatment; this may be achieved, for example, and not by way of limitation, by local infusion during surgery, topical application, e.g., by injection, by means of a catheter, or by means of an implant, the implant being of a porous, non-porous, or gelatinous material, including membranes, such as sialastic membranes, fibers, or commercial skin substitutes.

In another embodiment, the pharmaceutical composition can be delivered in a vesicle, in particular a liposome (see Langer, Science 249:1527-1533, 1990). In yet another embodiment, the active agent can be delivered in a controlled release system. In one embodiment, a pump may be used.

### Patient Groups

Patients who can benefit from the proposed treatment include patients recovering from acute or critical illness requiring intensive care or prolonged hospitalization (more than 1 week); frail elderly patients with sarcopenia; young adults recovering from severe trauma, such as motor vehicle accidents, severe burns, combat injuries, and other traumatic injuries; patients with chronic diseases known to cause cachexia, as listed above; and patients with muscle diseases, as listed above. Since loss of muscle is a common complication of most illnesses that are either severe or prolonged, it is anticipated that reversal of muscle wasting will speed the recovery and return to function of patients who experience muscle loss regardless of the root cause of this loss.

### Combination Therapy

This treatment may be combined with any treatment aimed at the primary cause of the muscle wasting process. Such combinations may include corticosteroids, immune suppressive agents, anti-cytokine agents, anti-cancer drugs; growth factors such as erythropoeitin, G-CSF, GM-CSF, or others; drugs used for the treatment of diabetes (including insulin and oral hypoglycemic agents), anti-tuberculosis drugs, and antibiotics. Combinations may include both small molecule and biomolecule agents.

The pharmaceutical compositions of the invention may be administered as the sole active agent or in conjunction with, e.g. as an adjuvant to or in combination to, other drugs e.g. an ActRIIB antibody, an ActRIIA antibody, a soluble ActRIIB decoy mimetic, an ActRIIA/B pan specific antibody an anti-myostatin antibody, a myostatin propeptide, a myostatin decoy protein that binds ActRIIB but does not activate it, a beta 2 agonist, a Ghrelin agonist, a SARM, GH agonists/mimetics or follistatin. For example, the drug of the invention may be used in combination with an ActRIIB antibody as disclosed in WO2010125003.

SEQUENCES

| SEQ | | | |
|---|---|---|---|
| ID | DNA/ | | |
| NO. | PROT. | Description | Sequence |
| 1 | PROT. | hIGF-1 | GPETLCGAELVDALQFVCGDRGFYFNKPTGYGSSSRRAPQTGIVDEC |
| | | without the | CFRSCDLRRLEMYCAPLKPAKSA |
| | | leader | |
| | | sequence | |
| 2 | PROT. | Ea peptide | RSVRAQRHTDMPKTQKEVHLKNASRGSAGNKNYRM |
| 3 | PROT. | Eb peptide | RSVRAQRHTDMPKTQKYQPPSTNKNTKSQRRKGWPKTHPGGEQKE |
| | | | GTEASLQIRGKKKEQRREIGSRNAECRGKKGK |
| 4 | PROT. | Ec peptide | RSVRAQRHTDMPKTQKYQPPSTNKNTKSQRRKGSTFEERK |
| 5 | PROT. | Wild type | GPETLCGAELVDALQFVCGDRGFYFNKPTGYGSSSRRAPQTGIVDEC |
| | | IGF-1-Ea | CFRSCDLRRLEMYCAPLKPAKSARSVRAQRHTDMPKTQKEVHLKNAS |
| | | without the | RGSAGNKNYRM |
| | | leader | |
| | | sequence | |
| 6 | PROT. | Example 6 | TLCGAELVDALQFVCGDRGFYFNKPTGYGSSSQRAPQTGIVDECCFR |
| | | | SCDLRRLEMYCAPLKPAKSAVRAQRHTDMPKTQKEVHLKNASRGSAG |
| | | | NKNYRM |
| 7 | PROT. | Example 7 | TLCGAELVDALQFVCGDRGFYFNKPTGYGSSSREAPQTGIVDECCFR |
| | | | SCDLRRLEMYCAPLKPAKSAVRAQRHTDMPKTQKEVHLKNASRGSAG |
| | | | NKNYRM |
| 8 | PROT. | Example 8 | TLCGAELVDALQFVCGDRGFYFNKPTGYGSSSRAAPQTGIVDECCFR |
| | | | SCDLRRLEMYCAPLKPAKSAVRAQRHTDMPKTQKEVHLKNASRGSAG |
| | | | NKNYRM |
| 9 | PROT. | Example 9 | TLCGAELVDALQFVCGDRGFYFNKPTGYGSSSRPAPQTGIVDECCFR |
| | | | SCDLRRLEMYCAPLKPAKSAVRAQRHTDMPKTQKEVHLKNASRGSAG |
| | | | NKNYRM |
| 10 | PROT. | Example 10 | TLCGAELVDALQFVCGDRGFYFNKPTGYGSSSQQAPQTGIVDECCFR |
| | | | SCDLRRLEMYCAPLKPAKSAVRAQRHTDMPKTQKEVHLKNASRGSAG |
| | | | NKNYRM |
| 11 | PROT. | Example 11 | TLCGAELVDALQFVCGDRGFYFNKPTGYGSSSQAAPQTSIVDECCFR |
| | | | SCDLRRLEMYCAPLKPAKSAVRAQRHTDMPKTQKEVHLKNASRGSAG |
| | | | NKNYRM |
| 12 | PROT. | Example 12 | TLCGAELVDALQFVCGDRGFYFNKPTGYGSSSQRAPQTSIVDECCFR |
| | | | SCDLRRLEMYCAPLKPAKSAVQAQRHTDMPKTQKEVHLKNASRGSAG |
| | | | NKNYRM |
| 13 | PROT. | Example 13 | TLCGAELVDALQFVCGDRGFYFNKPTGYGSSSQRAPQTSIVDECCFR |
| | | | SCDLRRLEMYCAPLKPAKSAVQAQQHTDMPKTQKEVHLKNASRGSA |
| | | | GNKNYRM |
| 14 | PROT. | Example 14 | TLCGAELVDALQFVCGDRGFYFNKPTGYGSSSQRAPQTSIVDECCFR |
| | | | SCDLRRLEMYCAPLKPAKSAVQAQQHTDMPKTQKEVHLKNASRGSA |
| | | | GNKNYQM |
| 15 | PROT. | Example 15 | TLCGAELVDALQFVCGDRGFYFNKPTGYGSSSREAPQTSIVDECCFRS |
| | | | CDLRRLEMYCAPLKPAKSAVRAQQHTDMPKTQKEVHLKNASRGSAG |
| | | | NKNYRM |
| 16 | PROT. | Example 16 | TLCGAELVDALQFVCGDRGFYFNKPTGYGSSSREAPQTSIVDECCFRS |
| | | | CDLRRLEMYCAPLKPAKSAVQAQQHTDMPKTQKEVHLKNASRGSAG |
| | | | NKNYRM |
| 17 | PROT. | Example 17 | TLCGAELVDALQFVCGDRGFYFNKPTGYGSSSREAPQTSIVDECCFRS |
| | | | CDLRRLEMYCAPLKPAKSAVQAQQHTDMPKTQKEVHLKNASRGSAG |
| | | | NKNYQM |
| 18 | PROT. | Example 18 | TLCGAELVDALQFVCGDRGFYFNKPTGYGSSSRAAPQTGIVDECCFR |
| | | | SCDLRRLEMYCAPLKPAKSAVRAQRHTDMPKTQKEVHLKNASRGSAG |
| | | | NKNYRM |
| 19 | PROT. | Example 19 | TLCGAELVDALQFVCGDRGFYFNKPTGYGSSSRAAPQTGIVDECCFR |
| | | | SCDLRRLEMYCAPLKPAKSAVQAQRHTDMPKTQKEVHLKNASRGSAG |
| | | | NKNYRM |
| 20 | PROT. | Example 20 | TLCGAELVDALQFVCGDRGFYFNKPTGYGSSSRAAPQTGIVDECCFR |
| | | | SCDLRRLEMYCAPLKPAKSAVQAQQHTDMPKTQKEVHLKNASRGSA |
| | | | GNKNYRM |
| 21 | PROT. | Example 21 | TLCGAELVDALQFVCGDRGFYFNKPTGYGSSSRAAPQTGIVDECCFR |
| | | | SCDLRRLEMYCAPLKPAKSAVQAQQHTDMPKTQKEVHLKNASRGSA |
| | | | GNKNYQM |
| 22 | PROT. | Example 22 | TLCGAELVDALQFVCGDRGFYFNKPTGYGSSSRPAPQTGIVDECCFR |
| | | | SCDLRRLEMYCAPLKPAKSAVQAQRHTDMPKTQKEVHLKNASRGSAG |
| | | | NKNYRM |
| 23 | PROT. | Example 23 | TLCGAELVDALQFVCGDRGFYFNKPTGYGSSSRPAPQTGIVDECCFR |
| | | | SCDLRRLEMYCAPLKPAKSAVQAQQHTDMPKTQKEVHLKNASRGSA |
| | | | GNKNYRM |
| 24 | PROT. | Example 24 | TLCGAELVDALQFVCGDRGFYFNKPTGYGSSSRPAPQTGIVDECCFR |
| | | | SCDLRRLEMYCAPLKPAKSAVQAQQHTDMPKTQKEVHLKNASRGSA |
| | | | GNKNYQM |
| 25 | PROT. | Example 25 | TLCGAELVDALQFVCGDRGFYFNKPTGYGSSSQQAPQTGIVDECCFR |
| | | | SCDLRRLEMYCAPLKPAKSAVQAQRHTDMPKTQKEVHLKNASRGSAG |
| | | | NKNYRM |
| 26 | PROT. | Example 26 | TLCGAELVDALQFVCGDRGFYFNKPTGYGSSSQQAPQTGIVDECCFR |
| | | | SCDLRRLEMYCAPLKPAKSAVQAQQHTDMPKTQKEVHLKNASRGSA |
| | | | GNKNYRM |
| 27 | PROT. | Example 27 | TLCGAELVDALQFVCGDRGFYFNKPTGYGSSSQAAPQTGIVDECCFR |
| | | | SCDLRRLEMYCAPLKPAKSAVQAQQHTDMPKTQKEVHLKNASRGSA |
| | | | GNKNYRM |
| 28 | PROT. | Example 28 | TLCGAELVDALQFVCGDRGFYFNKPTGYGSSSQQAPQTGIVDECCFR |
| | | | SCDLRRLEMYCAPLKPAKSAVQAQQHTDMPKTQKEVHLKNASRGSA |
| | | | GNKNYQM |
| 29 | PROT. | Example 29 | TLCGAELVDALQFVCGDRGFYFNKPTGYGSSSQAAPQTGIVDECCFR |
| | | | SCDLRRLEMYCAPLKPAKSAVQAQQHTDMPKTQKEVHLKNASRGSA |
| | | | GNKNYQM |
| 30 | PROT. | Example 30 | TLCGAELVDALQFVCGDRGFYFNKPTGYGSSSQRAPQTGIVDECCFR |
| | | | SCDLRRLEMYCAPLKPAVRAQRHTDMPKTQKEVHLKNASRGSAGNK |
| | | | NYRM |
| 31 | PROT. | Example 31 | TLCGAELVDALQFVCGDRGFYFNKPTGYGSSSREAPQTGIVDECCFR |
| | | | SCDLRRLEMYCAPLKPAVRAQRHTDMPKTQKEVHLKNASRGSAGNK |
| | | | NYRM |
| 32 | PROT. | Example 32 | TLCGAELVDALQFVCGDRGFYFNKPTGYGSSSRAAPQTGIVDECCFR |
| | | | SCDLRRLEMYCAPLKPAVRAQRHTDMPKTQKEVHLKNASRGSAGNK |
| | | | NYRM |
| 33 | PROT. | Example 33 | TLCGAELVDALQFVCGDRGFYFNKPTGYGSSSRPAPQTGIVDECCFR |
| | | | SCDLRRLEMYCAPLKPAVRAQRHTDMPKTQKEVHLKNASRGSAGNK |
| | | | NYRM |
| 34 | PROT. | Example 34 | TLCGAELVDALQFVCGDRGFYFNKPTGYGSSSQQAPQTGIVDECCFR |
| | | | SCDLRRLEMYCAPLKPAQVRAQRHTDMPKTQKEVHLKNASRGSAGN |
| | | | KNYRM |
| 35 | PROT. | Example 35 | TLCGAELVDALQFVCGDRGFYFNKPTGYGSSSQAAPQTGIVDECCFR |
| | | | SCDLRRLEMYCAPLKPAVRAQRHTDMPKTQKEVHLKNASRGSAGNK |
| | | | NYRM |
| 36 | PROT. | Example 36 | TLCGAELVDALQFVCGDRGFYFNKPTGYGSSSQRAPQTGIVDECCFR |
| | | | SCDLRRLEMYCAPLKPAVQAQRHTDMPKTQKEVHLKNASRGSAGNK |
| | | | NYRM |
| 37 | PROT. | Example 37 | TLCGAELVDALQFVCGDRGFYFNKPTGYGSSSQRAPQTGIVDECCFR |
| | | | SCDLRRLEMYCAPLKPAVQAQQHTDMPKTQKEVHLKNASRGSAGNK |
| | | | NYRM |
| 38 | PROT. | Example 38 | TLCGAELVDALQFVCGDRGFYFNKPTGYGSSSQRAPQTGIVDECCFR |
| | | | SCDLRRLEMYCAPLKPAVQAQQHTDMPKTQKEVHLKNASRGSAGNK |
| | | | NYQM |
| 39 | PROT. | Example 39 | TLCGAELVDALQFVCGDRGFYFNKPTGYGSSSREAPQTGIVDECCFR |
| | | | SCDLRRLEMYCAPLKPAVQAQRHTDMPKTQKEVHLKNASRGSAGNK |
| | | | NYRM |
| 40 | PROT. | Example 40 | TLCGAELVDALQFVCGDRGFYFNKPTGYGSSSREAPQTGIVDECCFR |
| | | | SCDLRRLEMYCAPLKPAVQAQQHTDMPKTQKEVHLKNASRGSAGNK |
| | | | NYRM |
| 41 | PROT. | Example 41 | TLCGAELVDALQFVCGDRGFYFNKPTGYGSSSREAPQTGIVDECCFR |
| | | | SCDLRRLEMYCAPLKPAVQAQQHTDMPKTQKEVHLKNASRGSAGNK |
| | | | NYQM |
| 42 | PROT. | Example 42 | TLCGAELVDALQFVCGDRGFYFNKPTGYGSSSRAAPQTGIVDECCFR |
| | | | SCDLRRLEMYCAPLKPAVQAQRHTDMPKTQKEVHLKNASRGSAGNK |
| | | | NYRM |
| 43 | PROT. | Example 43 | TLCGAELVDALQFVCGDRGFYFNKPTGYGSSSRAAPQTGIVDECCFR |
| | | | SCDLRRLEMYCAPLKPAVQAQQHTDMPKTQKEVHLKNASRGSAGNK |
| | | | NYRM |
| 44 | PROT. | Example 44 | TLCGAELVDALQFVCGDRGFYFNKPTGYGSSSRAAPQTGIVDECCFR |
| | | | SCDLRRLEMYCAPLKPAVQAQQHTDMPKTQKEVHLKNASRGSAGNK |
| | | | NYQM |
| 45 | PROT. | Example 45 | TLCGAELVDALQFVCGDRGFYFNKPTGYGSSSRPAPQTGIVDECCFR |
| | | | SCDLRRLEMYCAPLKPAVQAQRHTDMPKTQKEVHLKNASRGSAGNK |
| | | | NYRM |
| 46 | PROT. | Example 46 | TLCGAELVDALQFVCGDRGFYFNKPTGYGSSSRPAPQTGIVDECCFR |
| | | | SCDLRRLEMYCAPLKPAVQAQQHTDMPKTQKEVHLKNASRGSAGNK |
| | | | NYRM |
| 47 | PROT. | Example 47 | TLCGAELVDALQFVCGDRGFYFNKPTGYGSSSRPAPQTGIVDECCFR |
| | | | SCDLRRLEMYCAPLKPAVQAQQHTDMPKTQKEVHLKNASRGSAGNK |
| | | | NYQM |
| 48 | PROT. | Example 48 | TLCGAELVDALQFVCGDRGFYFNKPTGYGSSSQQAPQTGIVDECCFR |
| | | | SCDLRRLEMYCAPLKPAVQAQRHTDMPKTQKEVHLKNASRGSAGNK |
| | | | NYRM |
| 49 | PROT. | Example 49 | TLCGAELVDALQFVCGDRGFYFNKPTGYGSSSQQAPQTGIVDECCFR |
| | | | SCDLRRLEMYCAPLKPAVQAQQHTDMPKTQKEVHLKNASRGSAGNK |
| | | | NYRM |
| 50 | PROT. | Example 50 | TLCGAELVDALQFVCGDRGFYFNKPTGYGSSSQAAPQTGIVDECCFR |
| | | | SCDLRRLEMYCAPLKPAVQAQQHTDMPKTQKEVHLKNASRGSAGNK |
| | | | NYRM |
| 51 | PROT. | Example 51 | TLCGAELVDALQFVCGDRGFYFNKPTGYGSSSQQAPQTGIVDECCFR |
| | | | SCDLRRLEMYCAPLKPAVQAQQHTDMPKTQKEVHLKNASRGSAGNK |
| | | | NYQM |
| 52 | PROT. | Example 52 | TLCGAELVDALQFVCGDRGFYFNKPTGYGSSSQAAPQTGIVDECCFR |
| | | | SCDLRRLEMYCAPLKPAVQAQQHTDMPKTQKEVHLKNASRGSAGNK |
| | | | NYQM |
| 53 | PROT. | Example 53 | GPTLCGAELVDALQFVCGDRGFYFNKPTGYGSSSQRAPQTGIVDECC |
| | | | FRSCDLRRLEMYCAPLKPAKSAVRAQRHTDMPKTQKEVHLKNASRGS |
| | | | AGNKNYRM |
| 54 | PROT. | Example 54 | GPTLCGAELVDALQFVCGDRGFYFNKPTGYGSSSREAPQTGIVDECC |
| | | | FRSCDLRRLEMYCAPLKPAKSAVRAQRHTDMPKTQKEVHLKNASRGS |
| | | | AGNKNYRM |
| 55 | PROT. | Example 55 | GPTLCGAELVDALQFVCGDRGFYFNKPTGYGSSSRAAPQTGIVDECC |
| | | | FRSCDLRRLEMYCAPLKPAKSAVRAQRHTDMPKTQKEVHLKNASRGS |
| | | | AGNKNYRM |
| 56 | PROT. | Example 56 | GPTLCGAELVDALQFVCGDRGFYFNKPTGYGSSSRPAPQTGIVDECC |
| | | | FRSCDLRRLEMYCAPLKPAKSAVRAQRHTDMPKTQKEVHLKNASRGS |
| | | | AGNKNYRM |
| 57 | PROT. | Example 57 | GPTLCGAELVDALQFVCGDRGFYFNKPTGYGSSSQQAPQTGIVDECC |
| | | | FRSCDLRRLEMYCAPLKPAKSAVRAQRHTDMPKTQKEVHLKNASRGS |
| | | | AGNKNYRM |
| 58 | PROT. | Example 58 | GPTLCGAELVDALQFVCGDRGFYFNKPTGYGSSSQAAPQTGIVDECC |
| | | | FRSCDLRRLEMYCAPLKPAKSAVRAQRHTDMPKTQKEVHLKNASRGS |
| | | | AGNKNYRM |
| 59 | PROT. | Example 59 | GPTLCGAELVDALQFVCGDRGFYFNKPTGYGSSSQRAPQTGIVDECC |
| | | | FRSCDLRRLEMYCAPLKPAKSAVQAQRHTDMPKTQKEVHLKNASRGS |
| | | | AGNKNYRM |
| 60 | PROT. | Example 60 | GPTLCGAELVDALQFVCGDRGFYFNKPTGYGSSSQRAPQTGIVDECC |
| | | | FRSCDLRRLEMYCAPLKPAKSAVQAQQHTDMPKTQKEVHLKNASRGS |
| | | | AGNKNYRM |
| 61 | PROT. | Example 61 | GPTLCGAELVDALQFVCGDRGFYFNKPTGYGSSSQRAPQTGIVDECC |
| | | | FRSCDLRRLEMYCAPLKPAKSAVQAQQHTDMPKTQKEVHLKNASRGS |
| | | | AGNKNYQM |
| 62 | PROT. | Example 62 | GPTLCGAELVDALQFVCGDRGFYFNKPTGYGSSSREAPQTGIVDECC |
| | | | FRSCDLRRLEMYCAPLKPAKSAVRAQQHTDMPKTQKEVHLKNASRGS |
| | | | AGNKNYRM |
| 63 | PROT. | Example 63 | GPTLCGAELVDALQFVCGDRGFYFNKPTGYGSSSREAPQTGIVDECC |
| | | | FRSCDLRRLEMYCAPLKPAKSAVQAQQHTDMPKTQKEVHLKNASRGS |
| | | | AGNKNYRM |
| 64 | PROT. | Example 64 | GPTLCGAELVDALQFVCGDRGFYFNKPTGYGSSSREAPQTGIVDECC |
| | | | FRSCDLRRLEMYCAPLKPAKSAVQAQQHTDMPKTQKEVHLKNASRGS |
| | | | AGNKNYQM |
| 65 | PROT. | Example 65 | GPTLCGAELVDALQFVCGDRGFYFNKPTGYGSSSRAAPQTGIVDECC |
| | | | FRSCDLRRLEMYCAPLKPAKSAVQAQRHTDMPKTQKEVHLKNASRGS |
| | | | AGNKNYRM |
| 66 | PROT. | Example 66 | GPTLCGAELVDALQFVCGDRGFYFNKPTGYGSSSRAAPQTGIVDECC |
| | | | FRSCDLRRLEMYCAPLKPAKSAVQAQQHTDMPKTQKEVHLKNASRGS |
| | | | AGNKNYRM |
| 67 | PROT. | Example 67 | GPTLCGAELVDALQFVCGDRGFYFNKPTGYGSSSRAAPQTGIVDECC |
| | | | FRSCDLRRLEMYCAPLKPAKSAVQAQQHTDMPKTQKEVHLKNASRGS |
| | | | AGNKNYQM |
| 68 | PROT. | Example 68 | GPTLCGAELVDALQFVCGDRGFYFNKPTGYGSSSRPAPQTGIVDECC |
| | | | FRSCDLRRLEMYCAPLKPAKSAVQAQRHTDMPKTQKEVHLKNASRGS |
| | | | AGNKNYRM |
| 69 | PROT. | Example 69 | GPTLCGAELVDALQFVCGDRGFYFNKPTGYGSSSRPAPQTGIVDECC |
| | | | FRSCDLRRLEMYCAPLKPAKSAVQAQQHTDMPKTQKEVHLKNASRGS |
| | | | AGNKNYRM |
| 70 | PROT. | Example 70 | GPTLCGAELVDALQFVCGDRGFYFNKPTGYGSSSRPAPQTGIVDECC |
| | | | FRSCDLRRLEMYCAPLKPAKSAVQAQQHTDMPKTQKEVHLKNASRGS |
| | | | AGNKNYQM |
| 71 | PROT. | Example 71 | GPTLCGAELVDALQFVCGDRGFYFNKPTGYGSSSQQAPQTGIVDECC |
| | | | FRSCDLRRLEMYCAPLKPAKSAVQAQRHTDMPKTQKEVHLKNASRGS |
| | | | AGNKNYRM |
| 72 | PROT. | Example 72 | GPTLCGAELVDALQFVCGDRGFYFNKPTGYGSSSQQAPQTGIVDECC |
| | | | FRSCDLRRLEMYCAPLKPAKSAVQAQQHTDMPKTQKEVHLKNASRGS |
| | | | AGNKNYRM |
| 73 | PROT. | Example 73 | GPTLCGAELVDALQFVCGDRGFYFNKPTGYGSSSQAAPQTGIVDECC |
| | | | FRSCDLRRLEMYCAPLKPAKSAVQAQQHTDMPKTQKEVHLKNASRGS |
| | | | AGNKNYRM |
| 74 | PROT. | Example 74 | GPTLCGAELVDALQFVCGDRGFYFNKPTGYGSSSQQAPQTGIVDECC |
| | | | FRSCDLRRLEMYCAPLKPAKSAVQAQQHTDMPKTQKEVHLKNASRGS |
| | | | AGNKNYQM |
| 75 | PROT. | Example 75 | GPTLCGAELVDALQFVCGDRGFYFNKPTGYGSSSQAAPQTGIVDECC |
| | | | FRSCDLRRLEMYCAPLKPAKSAVQAQQHTDMPKTQKEVHLKNASRGS |
| | | | AGNKNYQM |
| 76 | PROT. | Example 76 | GPTLCGAELVDALQFVCGDRGFYFNKPTGYGSSSQRAPQTGIVDECC |
| | | | FRSCDLRRLEMYCAPLKPAVRAQRHTDMPKTQKEVHLKNASRGSAGN |
| | | | KNYRM |
| 77 | PROT. | Example 77 | GPTLCGAELVDALQFVCGDRGFYFNKPTGYGSSSREAPQTGIVDECC |
| | | | FRSCDLRRLEMYCAPLKPAVRAQRHTDMPKTQKEVHLKNASRGSAGN |
| | | | KNYRM |
| 78 | PROT. | Example 78 | GPTLCGAELVDALQFVCGDRGFYFNKPTGYGSSSRAAPQTGIVDECC |
| | | | FRSCDLRRLEMYCAPLKPAVRAQRHTDMPKTQKEVHLKNASRGSAGN |
| | | | KNYRM |
| 79 | PROT. | Example 79 | GPTLCGAELVDALQFVCGDRGFYFNKPTGYGSSSRPAPQTGIVDECC |
| | | | FRSCDLRRLEMYCAPLKPAVRAQRHTDMPKTQKEVHLKNASRGSAGN |
| | | | KNYRM |
| 80 | PROT. | Example 80 | GPTLCGAELVDALQFVCGDRGFYFNKPTGYGSSSQQAPQTGIVDECC |
| | | | FRSCDLRRLEMYCAPLKPAQVRAQRHTDMPKTQKEVHLKNASRGSA |
| | | | GNKNYRM |
| 81 | PROT. | Example 81 | GPTLCGAELVDALQFVCGDRGFYFNKPTGYGSSSQAAPQTGIVDECC |
| | | | FRSCDLRRLEMYCAPLKPAVRAQRHTDMPKTQKEVHLKNASRGSAGN |
| | | | KNYRM |
| 82 | PROT. | Example 82 | GPTLCGAELVDALQFVCGDRGFYFNKPTGYGSSSQRAPQTGIVDECC |
| | | | FRSCDLRRLEMYCAPLKPAVQAQRHTDMPKTQKEVHLKNASRGSAG |
| | | | NKNYRM |
| 83 | PROT. | Example 83 | GPTLCGAELVDALQFVCGDRGFYFNKPTGYGSSSQRAPQTGIVDECC |
| | | | FRSCDLRRLEMYCAPLKPAVQAQQHTDMPKTQKEVHLKNASRGSAG |
| | | | NKNYRM |
| 84 | PROT. | Example 84 | GPTLCGAELVDALQFVCGDRGFYFNKPTGYGSSSQRAPQTGIVDECC |
| | | | FRSCDLRRLEMYCAPLKPAVQAQQHTDMPKTQKEVHLKNASRGSAG |
| | | | NKNYQM |
| 85 | PROT. | Example 85 | GPTLCGAELVDALQFVCGDRGFYFNKPTGYGSSSREAPQTGIVDECC |
| | | | FRSCDLRRLEMYCAPLKPAVQAQRHTDMPKTQKEVHLKNASRGSAG |
| | | | NKNYRM |
| 86 | PROT. | Example 86 | GPTLCGAELVDALQFVCGDRGFYFNKPTGYGSSSREAPQTGIVDECC |
| | | | FRSCDLRRLEMYCAPLKPAVQAQQHTDMPKTQKEVHLKNASRGSAG |
| | | | NKNYRM |
| 87 | PROT. | Example 87 | GPTLCGAELVDALQFVCGDRGFYFNKPTGYGSSSREAPQTGIVDECC |
| | | | FRSCDLRRLEMYCAPLKPAVQAQQHTDMPKTQKEVHLKNASRGSAG |
| | | | NKNYQM |
| 88 | PROT. | Example 88 | GPTLCGAELVDALQFVCGDRGFYFNKPTGYGSSSRAAPQTGIVDECC |
| | | | FRSCDLRRLEMYCAPLKPAVQAQRHTDMPKTQKEVHLKNASRGSAG |
| | | | NKNYRM |
| 89 | PROT. | Example 89 | GPTLCGAELVDALQFVCGDRGFYFNKPTGYGSSSRAAPQTGIVDECC |
| | | | FRSCDLRRLEMYCAPLKPAVQAQQHTDMPKTQKEVHLKNASRGSAG |
| | | | NKNYRM |
| 90 | PROT. | Example 90 | GPTLCGAELVDALQFVCGDRGFYFNKPTGYGSSSRAAPQTGIVDECC |
| | | | FRSCDLRRLEMYCAPLKPAVQAQQHTDMPKTQKEVHLKNASRGSAG |
| | | | NKNYQM |
| 91 | PROT. | Example 91 | GPTLCGAELVDALQFVCGDRGFYFNKPTGYGSSSRPAPQTGIVDECC |
| | | | FRSCDLRRLEMYCAPLKPAVQAQRHTDMPKTQKEVHLKNASRGSAG |
| | | | NKNYRM |
| 92 | PROT. | Example 92 | GPTLCGAELVDALQFVCGDRGFYFNKPTGYGSSSRPAPQTGIVDECC |
| | | | FRSCDLRRLEMYCAPLKPAVQAQQHTDMPKTQKEVHLKNASRGSAG |
| | | | NKNYRM |
| 93 | PROT. | Example 93 | GPTLCGAELVDALQFVCGDRGFYFNKPTGYGSSSRPAPQTGIVDECC |
| | | | FRSCDLRRLEMYCAPLKPAVQAQQHTDMPKTQKEVHLKNASRGSAG |
| | | | NKNYQM |
| 94 | PROT. | Example 94 | GPTLCGAELVDALQFVCGDRGFYFNKPTGYGSSSQQAPQTGIVDECC |
| | | | FRSCDLRRLEMYCAPLKPAVQAQRHTDMPKTQKEVHLKNASRGSAG |
| | | | NKNYRM |
| 95 | PROT. | Example 95 | GPTLCGAELVDALQFVCGDRGFYFNKPTGYGSSSQQAPQTGIVDECC |
| | | | FRSCDLRRLEMYCAPLKPAVQAQQHTDMPKTQKEVHLKNASRGSAG |
| | | | NKNYRM |
| 96 | PROT. | Example 96 | GPTLCGAELVDALQFVCGDRGFYFNKPTGYGSSSQAAPQTGIVDECC |
| | | | FRSCDLRRLEMYCAPLKPAVQAQQHTDMPKTQKEVHLKNASRGSAG |
| | | | NKNYRM |
| 97 | PROT. | Example 97 | GPTLCGAELVDALQFVCGDRGFYFNKPTGYGSSSQQAPQTGIVDECC |
| | | | FRSCDLRRLEMYCAPLKPAVQAQQHTDMPKTQKEVHLKNASRGSAG |
| | | | NKNYQM |
| 98 | PROT. | Example 98 | GPTLCGAELVDALQFVCGDRGFYFNKPTGYGSSSQAAPQTGIVDECC |
| | | | FRSCDLRRLEMYCAPLKPAVQAQQHTDMPKTQKEVHLKNASRGSAG |
| | | | NKNYQM |
| 99 | PROT. | | VQAQQHTDMPKTQKEVHLKNASG |
| 100 | PROT. | | VQAQQHTDMPKTQKYQPPATNKNTKSQRRKGS |
| 101 | PROT. | | VQAQQHTDMPKTQKEVHLKNASRGSAGNKNYQM |

### EXAMPLES

The IGF-1Ea precursor protein possesses high number of surface exposed lysine residues. Only optimization of PEGylation parameters does not assure sufficient yield and especially reproducibility of the final product composition is not high enough. The yield of PEGylation reaction was increased by performing PEGylation reaction at pH 6.5. At this pH the selectivity of the reductive alkylation coupling of PEG-CHO reagent with IGF-1Ea is higher and the formation of side products is reduced. It was very surprising that PEGylation reaction performed at higher pH is more selective, because in the prior-art it is generally believed that reductive alkylation is more selective at lower pH.

### Part A

### General Methodology

### Process Description

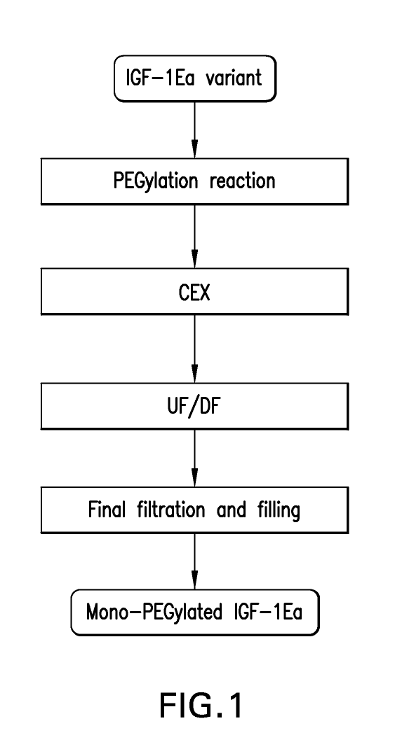

The process for preparation of mono-PEGylated IGF-1Ea precursor protein comprises the steps (i) PEGylation reaction, wherein the hIGF-1Ea precursor peptide is coupled with PEG reagent, (ii) chromatographic purification, wherein the desired product is purified from non-reacted PEG reagents, non-PEGylated IGF-1Ea precursor proteins and reaction by-products, and (iii) ultra/diafiltration step, wherein the buffer is exchanged and the pegylated protein of interest is concentrated. The process flow diagram is presented in FIG. 1.

Preparation of hIGF-1Ea Protein Using the N-Pro Technology.

A DNA expression vector encoding the hIGF-1-Ea precursor polypeptide containing the following modifications was constructed: deletion of E3; mutation of R37 to A; and deletion of R71 and deletion of S72 (SEQ ID NO: 55).

hIGF-1-Ea variant SEQ ID NO: 55 (ΔE3; R37A; ΔR71, ΔS72) gptlcgaelvdalgfvcgdrgfyfnkptgygsssraapgtgivdeccfrscdlrrlemycaplkpaksavragrhtdmpktqkevhlknasrgsagnknyrm (SEQ ID NO: 55)

The above described hIGF-1-Ea precursor polypeptide of SEQ ID NO: 55 was produced using the Npro Autoprotease Fusion Technology (NAFT). The skilled person is aware of this technology which, inter alia, has been disclosed in patent applications/patents WO200111056, WO200111057, WO2006113957, EP1200604B1 and U.S. Pat. No. 6,936,455B1.

### PEGylation Reaction

During PEGylation reaction the hIGF-1-Ea precursor polypeptide of SEQ ID: 55 is coupled with 30 kDa PEG aldehyde reagent (PEG-CHO) under reductive conditions achieved by NaCNBH₃. It is known from the literature that PEG-CHO reagent predominantly reacts with N-terminal amino group. High number of surface exposed lysine residues on the hIGF-1-Ea precursor polypeptide of the SEQ ID: 55 molecules reduce selectivity of N-terminal PEGylation by PEG-CHO. By performing PEGylation reaction at different pH values it was found that the reaction is more selective for N-terminal at pH 6.5 compared to pH 4.0 This is a surprising result since it is generally believed that PEGylation reaction using PEG-CHO is more selective at lower pH (Kinstler et al, Advanced Drug Delivery Reviews 54 (2002) 477-485, Molineux, Current Pharmaceutical Design, 10 (2004) 1235-1244).

Addition of α-CD to the PEGylation mixture provides conditions where the formation of higher PEGylated variants (namely di-, tri- or higher PEGylated) is suppressed.

### Chromatographic Purification

Mono-PEGylated hIGF-1-Ea precursor polypeptides of SEQ ID: 55 are purified from excess reagents, reaction by-products and non-PEGylated hIGF-1-Ea precursor polypeptide of SEQ ID: 55 by chromatography on strong cation exchange resin. The purification is performed at a pH which still enables retention of mono-PEGylated hIGF-1-Ea precursor polypeptide of SEQ ID: 55 on the column and is at the same time high enough to achieve at least partial separation between Lysine and N-terminally mono-PEGylated hIGF-1-Ea precursor polypeptide of SEQ ID: 55. pH 6.5 was determined to be the most appropriate for the separation as at higher pH the mono-PEGylated hIGF-1-Ea precursor polypeptides of SEQ ID: 55 does not bind to the column and at lower pH (eg. 5.0) there is no difference in retention between lysine and N-terminally PEGylated hIGF-1-Ea precursor polypeptide of SEQ ID:55.

### Concentration and Buffer Exchange

The UF/DF step is performed to exchange buffer into the final formulation buffer and to concentrate IGF-1Ea to the final concentration.

### Part B

### Working Examples

### Example 1

This example demonstrates the influence of PEGylation reaction pH on the selectivity of the PEGylation reaction by PEG-CHO. Surprisingly, at higher pH the reaction is more selective for N-terminal conjugation compared to the lower pH.

PEGylation of hIGF-1-Ea Precursor Polypeptide of SEQ ID: 55

PEGylation reactions were performed in two different buffers with pH 4.0 and 6.5. For lower pH, 20 mM sodium acetate, 50 mM Sodium chloride pH 4.0 was selected, while for higher pH, 50 mM Sodium Phosphate, 140 mM Sodium chloride pH 6.5 was used. In both cases solid PEG reagent (30 kDa polyethylene glycol propionaldehyde), solid α-CD and solid NaCNBH₃ were added to the IGF-1-Ea precursor polypeptide of SEQ ID: 55 solution. The PEGylation reactions were performed at room temperature, without steering, at protein concentration 4.8 mg/ml, at 3 molar excess of PEG regent, at 3% concentration of α-CD and at 40 mM concentration of NaCNBH₃. A sample for the analysis was withdrawn after 3.5 and 7 h of PEGylation. The samples were analyzed with reversed phase chromatography (RP-HPLC) and cation exchange chromatography (CE-HPLC).

### Characterization of the PEGylation Mixture

RP-HPLC was used for the determination of the amount of mono- and higher PEGylated forms while CE-HPLC was used for the determination of the % of N-terminally PEGylated forms in monopegylated fractions. With regard to composition of each sample, results demonstrate that the PEGylation reaction is more selective for N-terminal conjugation at pH 6.5 than at pH 4.0. At pH 6.5 the amount of N-terminally PEGylated forms is about 10% higher compared to the pH 4.0. Also the amount of higher PEGylated forms is significantly lower at higher pH. After seven hours of PEGylation the amounts of higher PEGylated forms at pH 6.5 is 16% and at pH 4.0 29%, while percentage of mono-PEGylated forms is the same after 7 hours independently on pH of PEGylation reaction. The results are presented in Table 1.

TABLE 1
Comparison of reaction mixture compositions at different pH values

| | pH 4.0 | | | pH 6.5 | | |
|---|---|---|---|---|---|---|
| | | | N-term in | | | N-term in |
| PEGylation | monoPEG* | higherPEG* | monoPEG** | monoPEG* | higherPEG* | monoPEG** |
| time | [Area %] | [Area %] | [Area %] | [Area %] | [Area %] | [Area %] |
| 3.5 h | 44 | 22 | 59 | 41 | 12 | 66 |
| 7 h | 44 | 29 | 60 | 44 | 16 | 67 |

*determined by RP-HPLC;
**determined by CE-HPLC

### Example 2

This example demonstrates the influence of α-CD on the amount of higher PEGylated forms. The amount of higher PEGylated forms is reduced in the presence of α-CD.

PEGylation of hIGF-1-Ea Precursor Polypeptide of SEQ ID: 55

Two PEGylation reactions were performed. In both cases solid PEG reagent (30 kDa polyethylene glycol propionaldehyde; NOF) and solid NaCNBH₃ were added to the IGF-1Ea solution. In one reaction solid α-CD was added up to 3% final concentration. The PEGylation reactions were performed at room temperature, without steering, at protein concentration of 4.4 mg/ml, at 4 molar excess of PEG regent and at 40 mM concentration of NaCNBH₃. A sample for the analysis was withdrawn after 2 h and 3.5 h of PEGylation. The samples were analyzed with RP-HPLC and CE-HPLC.

### Characterization of the PEGylation Mixture

The PEGylation mixtures were analyzed after 2 and 3.5 h with RP-HPLC. RP-HPLC was used for the determination of the amount of mono- and higher PEGylated forms.

The results demonstrate that the amount of higher PEGylated forms is lower in the presence of 3% α-CD. About 24% less higher PEGylated forms are obtained in the presence of 3% α-CD in the PEGylation mixture after 3.5 hours, while the percentage of mono-PEGylated forms remains the same. The results are presented in Table 2.

TABLE 2
Comparison of reaction mixture compositions in the
presence of α-CD and without α-CD addition

| | 0% α-CD | | 3% α-CD | |
|---|---|---|---|---|
| PEGylation | monoPEG | higherPEG | monoPEG | higherPEG |
| time | [%] | [%] | [%] | [%] |
| 2 h | 48 | 27 | 47 | 19 |
| 3.5 h | 47 | 33 | 47 | 25 |

### Example 3

### Partial Separation of Mono-PEGylated

### hIGF-1-Ea Precursor Polypeptide Isoforms of SEQ ID: 55

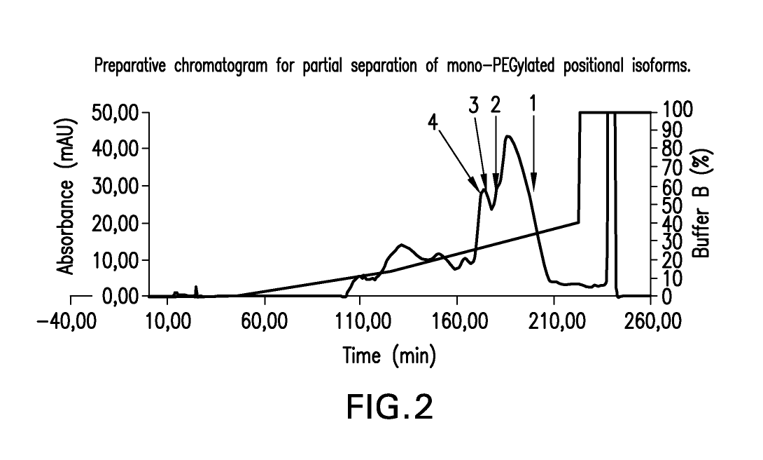

Solid PEG reagent (30 kDa polyethylene glycol propionaldehyde; NOF), neutralized solution of 5 M NaCNBH₃/1 M NaOH and 12% α-CD solution were added to the hIGF-1-Ea precursor polypeptide of SEQ ID NO: 55 (75 mg) solution. The PEGylation reaction was performed at pH 6.5, room temperature, protein concentration 6 mg/ml, 2.25 molar excess of PEG reagent, 50 mM concentration of NaCNBH₃ and at 3% concentration of α-CD. After 20 h the PEGylation mixture was diluted with water to lower sample conductivity and sterile filtrated. The purification and isolation of mono-PEGylated hIGF-1-Ea precursor polypeptide of SEQ ID NO: 55 isoforms was performed using cation exchange chromatography. The reaction mixture was loaded onto a 49.6 ml (XK 16/40, GE Healthcare) Toyopearl SP-650S (Tosoh bioscience) column, equilibrated in buffer (10 mM Na-phosphate, pH 6.5). The column was washed with 3 column volumes of equilibration buffer. The peptide forms were eluted using linear gradient from 0 to 100% of elution buffer (20 mM Na-phosphate, 500 mM naCl, pH 5.0) in 30 column volumes. The flow rate for the entire run was maintained at 2.5 ml/min. Preparative chromatogram is shown in FIG. 2.

During elution 2.5 ml fractions were collected. Fractions were pooled according to the analytical CE-HPLC. Four pools containing different composition of mono-PEGylated isoforms were prepared and designated as 1, 2, 3 and 4. The pools were concentrated and diafiltrated to the storage buffer, 20 mM acetate, pH 5.0. Concentration and diafiltration was performed on 15 ml Amicon centrifugal units with 3 kDa cut off.

The isoform composition (determined with analytical CE-HPLC) and the content of mono-PEGylated material (determined with RP-HPLC) in each pool is presented in Table 3.

TABLE 3
Comparison of sample compositions determined by RP-HPLC and CE-HPLC

| | RP-HPLC mono- | CE-HPLC | CE-HPLC | CE-HPLC | CE-HPLC | CE-HPLC |
|---|---|---|---|---|---|---|
| | PEG | lysine 3 | lysine 2a | lysine 2 | lysine 1 | N |
| CEX pools | IGF-1 Ea [Area %] | [Area %] | [Area %] | [Area %] | [Area %] | [Area %] |
| 1 | 97.2 | / | / | / | / | 100 |
| 2 | 93.6 | 10.7 | / | 48.7 | 34.5 | / |
| 3 | 94.7 | 23.8 | 36.0 | 32.5 | 4.6 | / |
| 4 | 95.6 | 16.1 | 79.8 | / | / | / |

Pool designated as 1 contains only N-terminally PEGylated hIGF-1-Ea precursor polypeptide of SEQ ID: 55. The amount of mono-PEGylated material is over 97%.

The pools designated as 2, 3 and 4 possesses lower RP-HPLC purity due to presence of some di-PEGylated material. The remaining mono-PEGylated material in pools 2, 3 and 4 is lysine-PEGylated. Since only partial separation of lysine-PEGylated isoforms was achieved on CEX chromatography the pools are mixtures of different lysine-PEGylated isoforms. On analytical CE-HPLC four peaks can be resolved. By taking into account the number of exposed lysine residues in hIGF-1-Ea precursor polypeptides of SEQ ID: 55 these four analytical CE-HPLC peaks are still mixtures of different lysine-PEGylated isoforms.

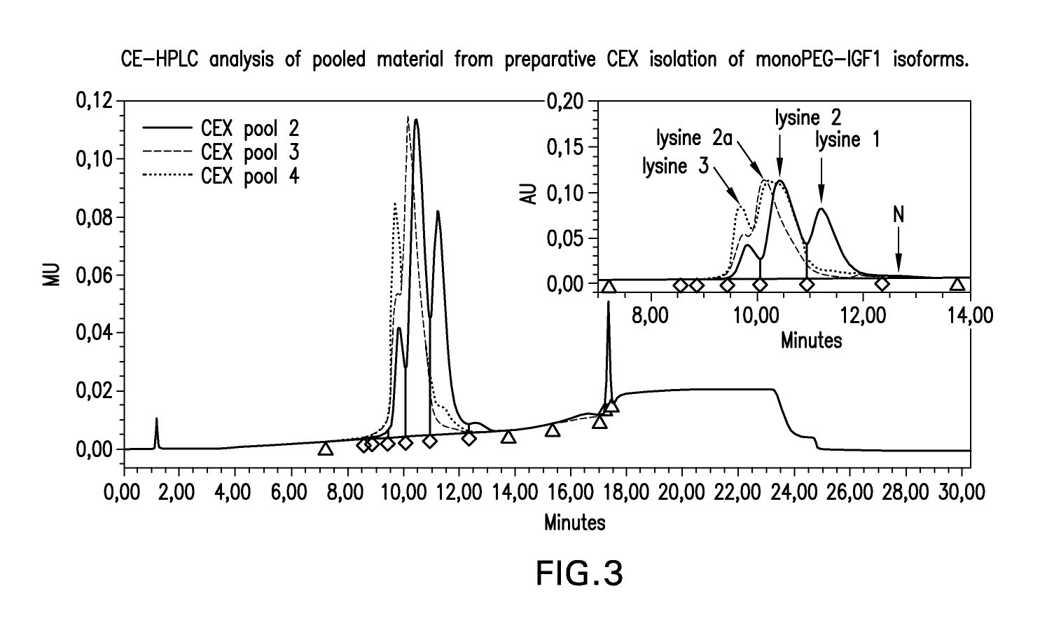

The CE-HPLC analysis of CEX pools designated as 2, 3 and 4 is shown in FIG. 3.

### Example 4

This example describes the process for the preparation of PEGylated hIGF-1-Ea precursor polypeptide of SEQ ID: 55. The whole process enables preparation of PEGylated hIGF-1-Ea precursor polypeptide of SEQ ID: 55 in a highly reproducible manner. The steps which were designed to increase process robustness were PEGylation reaction and CEX purification. α-CD was introduced in to the PEGylation reaction since it reduces formation of higher PEGylated forms and slows down the reaction so that the window for the PEGylation reaction termination (application on the CEX column) is wider and more appropriate for the large scale processes. The specificity of the reaction was improved by performing PEGylation reaction at pH 6.5. Additionally, the composition of the final product is controlled by purification by CEX. CEX was designed in a way where partial separation between lysine- and N-terminally PEGylated molecules is achieved. The consistency of the final product is ensured by appropriate pooling of CEX fraction. IPC control for pooling criteria to ensure consistent composition of isoforms is performed by analytical CE-HPLC.

### Preparation of PEG-Reagent and Reducing Agent

3.35 g hIGF-1-Ea precursor polypeptide of SEQ ID: 55 was thawed, thermostated to room temperature and homogenised by gentle mixing. PEG reagent (30 kDa polyethylene glycol propionaldehyde; NOF) was prepared as a 200 mg/ml stock solution in 20 mM Na-phosphate, 30 mM NaCl, pH 6.5. 12% solution of α-CD was prepared by dissolving α-CD in 140 mM NaH₂PO₄. 5 M NaCNBH₃ in 1 M NaOH was added to 12% α-CD solution to obtain 191 mM stock solution of NaCNBH3.

### PEGylation Reaction

Dissolved PEG-CHO reagent was added to the hIGF-1-Ea precursor polypeptide of SEQ ID: 55 solution. The PEGylation reaction was started by addition of α-CD and NaCNBH₃ solution. PEGylation reaction took place for 20.5 hours at 22° C., pH 6.4, protein concentration 6 mg/ml, 2.25 molar excess of PEG reagent, 50 mM concentration of NaCNBH₃ and at 3% concentration of α-CD.

### Chromatographic Purification

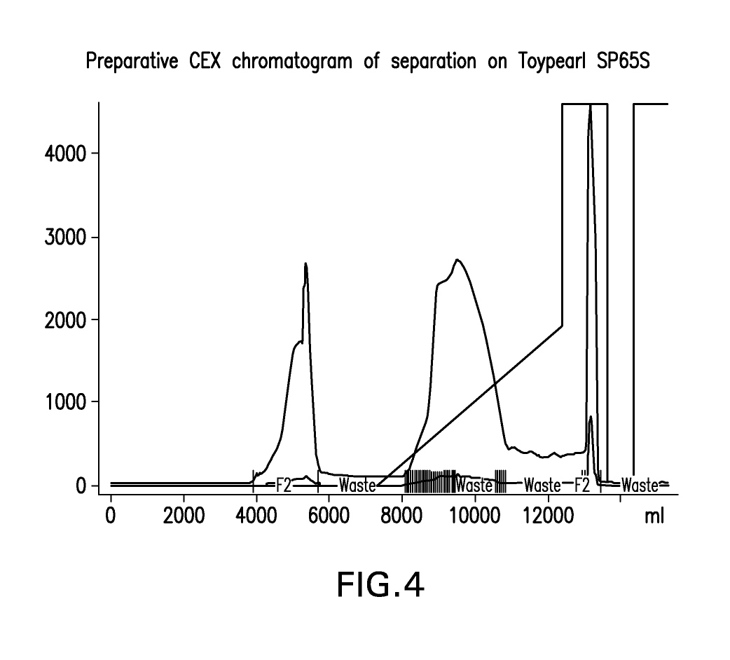

To achieve binding to CEX column, PEGylation mixture was diluted 2-fold with water. Diluted reaction mixture was loaded onto a XK 50/30 column (GE Healthcare, φ5 cm) Toyopearl SP-650S (Tosoh bioscience) column, equilibrated in buffer (20 mM Na-phosphate, pH 6.5). The column was washed with 3 column volumes of equilibration buffer. The peptide forms were eluted using linear gradient from 0 to 40% of elution buffer (20 mM Na-phosphate, 500 mM naCl, pH 5.0) in 5 column volumes. The flow rate for the entire run was maintained at 75 cm/h. Elution peak was fractionated and pooled according to the analytical CE-HPLC and RP-HPLC. Typical CEX chromatogram is shown in FIG. 4.

### Pooling

Pooling of fractions is performed according to the RP-HPLC and CE-HPLC analysis. The pool composition is shown in Table 4.

### UF/DF Step, Sterile Filtration and Filling

Ultrafiltration and diafiltration step was performed to concentrate PEGylated hIGF-1-Ea precursor polypeptide of SEQ ID: 55 and to exchange buffer. The buffer was exchanged into 7 mM Na-succinate, pH 5.0. For ultrafiltration and diafiltration three Pellicon Biomax 5 (Millipore) membranes were used. The diafiltration was performed at protein concentration between 6 and 7 mg/ml, with eight turn over volumes. Concentrated sample after UF/DF step was sterile filtrated using polyethersulfone membrane filter with 0.2 μm pore sizes and filled in to final storage containers (Nalgene 25 ml PETG bottles).

TABLE 4
Sample compositions determined by RP-HPLC and CE-HPLC

| | RP-HPLC | CE-HPLC |
|---|---|---|
| | mono-PEGylated | N-terminally PEGylated |
| | hIGF-1-Ea precursor | hIGF-1-Ea precursor |
| | polypeptide of SEQ | polypeptide of SEQ ID: 55 |
| | ID: 55 [Area %] | [Area %] |
| PEGylation mixture | 45.0 | 67.2 |
| CEX pool | 97.51 | 70.57 |
| Final product | 98.28 | 70.67 |

### Example 5

This example demonstrates purification of hIGF-1-Ea precursor polypeptide of SEQ ID NO: 55 PEGylation mixtures on a SP-Sepharose HP column.

### PEGylation Reaction

0.3 g hIGF-1-Ea precursor polypeptide of SEQ ID: 55 was thawed, thermostated to room temperature and homogenised by gentle mixing. Solid PEG reagent (polyethylene glycol propionaldehyde; NOF) and solid α-CD were added to the hIGF-1-Ea precursor polypeptide of SEQ ID: 55 solution. The PEGylation reaction was started by addition of NaCNBH₃ stock solution. PEGylation reaction took place for 3 hours at 22° C., pH 6.5, protein concentration 4.75 mg/ml, 3 molar excess of PEG reagent, 40 mM concentration of NaCNBH₃ and at 3% concentration of α-CD.

### Separation

For head to head comparison of separation on different CEX resins (Toyopearl SP 650S and SP Sepharose HP) larger amount of PEGylation mixture was prepared enabling several separation runs. PEGylation reaction was terminated by applying it to TSK gel SP-5PW column (removal of NaCNBH3 and removal of PEG) and brought back to original PEGylation buffer 10 mM Na-phosphate, pH 6.5 (using Amicon ultra centrifugal units with 5.000 kDa cut-off).

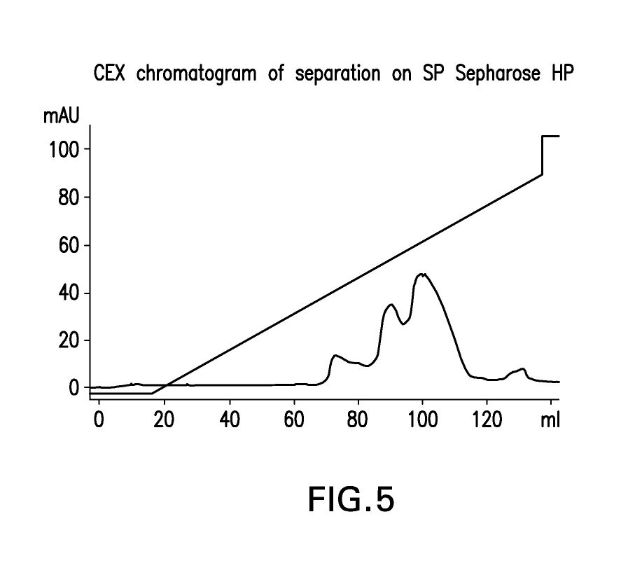

22.8 mg of hIGF-1-Ea precursor polypeptide of SEQ ID: 55 was loaded on to Tricorn 10/100 (GE Healthcare, φ1 cm) SP Sepharose HP (GE Healthcare) column, equilibrated in buffer (10 mM Na-phosphate, pH 6.5). The column was washed with 3 column volumes of equilibration buffer. The peptide forms were eluted using linear gradient from 0 to 85% of elution buffer (40 mM Na-phosphate, 200 mM naCl, pH 8.5) in 15 column volumes. The flow rate for the entire run was maintained at 0.9 ml/min. Elution peak was fractionated and pooled according to the analytical CE-HPLC and RP-HPLC. CEX chromatogram is shown in FIG. 5.

In addition to the above described hIGF-1-Ea precursor polypeptides variants, the following additional protein variants, without being limited to them, can be pegylated in accordance with the inventive methods described above:

### Example 6

G1, P2, E3 are deleted, amino acid R36 is substituted by glutamine (Q) and the amino acids R71 and S72 are deleted.

(SEQ ID NO: 6)
tlcgaelvdalqfvcgdrgfyfnkptgygsssqrapqtgivdeccfrscd
lrrlemycaplkpaksavraqrhtdmpktqkevhlknasrgsagnknyr
m.

### Example 7

G1, P2, E3 are deleted, amino acid R37 is substituted by glutamic acid (E) and the amino acids R71 and S72 are deleted.

(SEQ ID NO: 7)
tlcgaelvdalqfvcgdrgfyfnkptgygsssreapqtgivdeccfrscd
lrrlemycaplkpaksavraqrhtdmpktqkevhlknasrgsagnknyr
m.

### Example 8

G1, P2, E3 are deleted, amino acid R37 is substituted by alanine and the amino acids R71 and S72 are deleted.

(SEQ ID NO: 8)
tlcgaelvdalqfvcgdrgfyfnkptgygsssraapqtgivdeccfrscd
lrrlemycaplkpaksavraqrhtdmpktqkevhlknasrgsagnknyr
m.

### Example 9

G1, P2, E3 are deleted, amino acid R37 is substituted by proline (P) and the amino acids R71 and S72 are deleted.

(SEQ ID NO: 9)
tlcgaelvdalqfvcgdrgfyfnkptgygsssrpapqtgivdeccfrscd
lrrlemycaplkpaksavraqrhtdmpktqkevhlknasrgsagnknyr
m.

### Example 10

G1, P2, E3 are deleted, amino acid R36 and R37 are both substituted by glutamine (Q) and the amino acids R71 and S72 are deleted.

(SEQ ID NO: 10)
tlcgaelvdalqfvcgdrgfyfnkptgygsssqqapqtgivdeccfrscd
lrrlemycaplkpaksavraqrhtdmpktqkevhlknasrgsagnknyr
m.

### Example 11

G1, P2, E3 are deleted, amino acid R36 is substituted by glutamine (Q), R37 is substituted by alanine and the amino acids R71 and S72 are deleted.

(SEQ ID NO: 11)
tlcgaelvdalqfvcgdrgfyfnkptgygsssqaapqtgivdeccfrscd
lrrlemycaplkpaksavraqrhtdmpktqkevhlknasrgsagnknyr
m.

### Example 12

G1, P2, E3 are deleted, amino acid R36 is substituted by glutamine (Q) and the amino acids R71 and S72 are deleted and amino acid R74 is mutated to glutamine (Q).

(SEQ ID NO: 12)
tlcgaelvdalqfvcgdrgfyfnkptgygsssqrapqtgivdeccfrscd
lrrlemycaplkpaksavqaqrhtdmpktqkevhlknasrgsagnknyr
m.

### Example 13

G1, P2, E3 are deleted, amino acid R36 is substituted by glutamine (Q) and the amino acids R71 and S72 are deleted and amino acids R74 and R77 are mutated to glutamine (Q).

(SEQ ID NO: 13)
tlcgaelvdalqfvcgdrgfyfnkptgygsssqrapqtgivdeccfrscd
lrrlemycaplkpaksavqaqqhtdmpktqkevhlknasrgsagnknyr
m.

### Example 14

G1, P2, E3 are deleted, amino acid R36 is substituted by glutamine (Q) and the amino acids R71 and S72 are deleted and amino acids R74, R77 and R104 are mutated to glutamine (Q).

(SEQ ID NO: 14)
tlcgaelvdalqfvcgdrgfyfnkptgygsssqrapqtgivdeccfrscd
lrrlemycaplkpaksavqaqqhtdmpktqkevhlknasrgsagnknyq
m.

### Example 15

G1, P2, E3 are deleted, amino acid R37 is substituted by glutamic acid (E) and the amino acids R71 and S72 are deleted and amino acid R77 is mutated to glutamine (Q).

(SEQ ID NO: 15)
tlcgaelvdalqfvcgdrgfyfnkptgygsssreapqtgivdeccfrscd
lrrlemycaplkpaksavraqqhtdmpktqkevhlknasrgsagnknyr
m.

### Example 16

G1, P2, E3 are deleted, amino acid R37 is substituted by glutamic acid (E) and the amino acids R71 and S72 are deleted and amino acids R74 and R77 are mutated to glutamine (Q).

(SEQ ID NO: 16)
tlcgaelvdalqfvcgdrgfyfnkptgygsssreapqtgivdeccfrscd
lrrlemycaplkpaksavqaqqhtdmpktqkevhlknasrgsagnknyr
m.

### Example 17

G1, P2, E3 are deleted, amino acid R37 is substituted by glutamic acid (E) and the amino acids R71 and S72 are deleted and amino acids R74, R77 and R104 are mutated to glutamine (Q).

(SEQ ID NO: 17)
tlcgaelvdalqfvcgdrgfyfnkptgygsssreapqtgivdeccfrscd
lrrlemycaplkpaksavqaqqhtdmpktqkevhlknasrgsagnknyq
m.

### Example 18

G1, P2, E3 are deleted, amino acid R37 is substituted by alanine (A) and the amino acids R71 and S72 are deleted.

(SEQ ID NO: 18)
tlcgaelvdalqfvcgdrgfyfnkptgygsssraapqtgivdeccfrscd
lrrlemycaplkpaksavraqrhtdmpktqkevhlknasrgsagnknyr
m.

### Example 19

G1, P2, E3 are deleted, amino acid R37 is substituted by alanine (A) and the amino acids R71 and S72 are deleted and amino acid R74 is mutated to glutamine (Q).

(SEQ ID NO: 19)
tlcgaelvdalqfvcgdrgfyfnkptgygsssraapqtgivdeccfrscd
lrrlemycaplkpaksavqaqrhtdmpktqkevhlknasrgsagnknyr
m.

### Example 20

G1, P2, E3 are deleted, amino acid R37 is substituted by alanine (A) and the amino acids R71 and S72 are deleted and amino acids R74 and R77 are mutated to glutamine (Q).

(SEQ ID NO: 20)
tlcgaelvdalqfvcgdrgfyfnkptgygsssraapqtgivdeccfrscd
lrrlemycaplkpaksavqaqqhtdmpktqkevhlknasrgsagnknyr
m.

### Example 21

G1, P2, E3 are deleted, amino acid R37 is substituted by alanine (A) and the amino acids R71 and S72 are deleted and amino acids R74, R77 and R104 are mutated to glutamine (Q).

(SEQ ID NO: 21)
tlcgaelvdalqfvcgdrgfyfnkptgygsssraapqtgivdeccfrscd
lrrlemycaplkpaksavqaqqhtdmpktqkevhlknasrgsagnknyq
m.

### Example 22

G1, P2, E3 are deleted, amino acid R37 is substituted by proline (P) and the amino acids R71 and S72 are deleted and amino acid R74 is mutated to glutamine (Q).

(SEQ ID NO: 22)
tlcgaelvdalqfvcgdrgfyfnkptgygsssrpapqtgivdeccfrscd
lrrlemycaplkpaksavqaqrhtdmpktqkevhlknasrgsagnknyr
m.

### Example 23

G1, P2, E3 are deleted, amino acid R37 is substituted by proline (P) and the amino acids R71 and S72 are deleted and amino acids R74 and R77 are mutated to glutamine (Q).

(SEQ ID NO: 23)
tlcgaelvdalqfvcgdrgfyfnkptgygsssrpapqtgivdeccfrscd
lrrlemycaplkpaksavqaqqhtdmpktqkevhlknasrgsagnknyr
m.

### Example 24

G1, P2, E3 are deleted, amino acid R37 is substituted by proline (P) and the amino acids R71 and S72 are deleted and amino acids R74, R77 and R104 are mutated to glutamine (Q).

(SEQ ID NO: 24)
tlcgaelvdalqfvcgdrgfyfnkptgygsssrpapqtgivdeccfrscdl
rrlemycaplkpaksavqaqqhtdmpktqkevhlknasrgsagnknyqm.

### Example 25

G1, P2, E3 are deleted, amino acid R36 and R37 are both substituted by glutamine (Q) and the amino acids R71 and S72 are deleted and amino acid R74 is mutated to glutamine (Q).

(SEQ ID NO: 25)
tlcgaelvdalqfvcgdrgfyfnkptgygsssqqapqtgivdeccfrscdl
rrlemycaplkpaksavqaqrhtdmpktqkevhlknasrgsagnknyrm.

### Example 26

G1, P2, E3 are deleted, amino acid R36 and R37 are both substituted by glutamine (Q) and the amino acids R71 and S72 are deleted and amino acids R74 and R77 are mutated to glutamine (Q).

(SEQ ID NO: 26)
tlcgaelydalgfycgdrgfyfnkptgygsssqqapqtgivdeccfrscdl
rrlemycaplkpaksavqaqqhtdmpktqkevhlknasrgsagnknyrm.

### Example 27

G1, P2, E3 are deleted, amino acid R36 is substituted by glutamine (Q), R37 is substituted by alanine and the amino acids R71 and S72 are deleted and amino acids R74 and R77 are mutated to glutamine (Q).

(SEQ ID NO: 27)
tlcgaelvdalqfvcgdrgfyfnkptgygsssqaapqtgivdeccfrscdl
rrlemycaplkpaksavqaqqhtdmpktqkevhlknasrgsagnknyrm.

### Example 28

G1, P2, E3 are deleted, amino acid R36 and R37 are both substituted by glutamine (Q) and the amino acids R71 and S72 are deleted and amino acids R74, R77 and R104 are mutated to glutamine (Q).

(SEQ ID NO: 28)
tlcgaelvdalqfvcgdrgfyfnkptgygsssqqapqtgivdeccfrscdl
rrlemycaplkpaksavqaqqhtdmpktqkevhlknasrgsagnknyqm.

### Example 29

G1, P2, E3 are deleted, amino acid R36 is substituted by glutamine (Q), R37 is substituted by alanine and the amino acids R71 and S72 are deleted and amino acids R74, R77 and R104 are mutated to glutamine (Q).

(SEQ ID NO: 29)
tlcgaelvdalqfvcgdrgfyfnkptgygsssqaapqtgivdeccfrscdl
rrlemycaplkpaksavqaqqhtdmpktqkevhlknasrgsagnknyqm.

### Example 30

G1, P2, E3 are deleted, amino acid R36 is substituted by glutamine (Q) and the amino acids K68, S69, A70, R71 and S72 are deleted.

(SEQ ID NO: 30)
tlcgaelvdalqfvcgdrgfyfnkptgygsssqrapqtgivdeccfrscd
lrrlemycaplkpavraqrhtdmpktqkevhlknasrgsagnknyrm.

### Example 31

G1, P2, E3 are deleted, amino acid R37 is substituted by glutamic acid (E) and the amino acids K68, S69, A70, R71 and S72 are deleted.

(SEQ ID NO: 30)
tlcgaelvdalqfvcgdrgfyfnkptgygsssreapqtgivdeccfrscd
lrrlemycaplkpavraqrhtdmpktqkevhlknasrgsagnknyrm.

### Example 32

G1, P2, E3 are deleted, amino acid R37 is substituted by alanine and the amino acids K68, S69, A70, R71 and S72 are deleted.

(SEQ ID NO: 32)
tlcgaelvdalqfvcgdrgfyfnkptgygsssraapqtgivdeccfrscd
lrrlemycaplkpavraqrhtdmpktqkevhlknasrgsagnknyrm.

### Example 33

G1, P2, E3 are deleted, amino acid R37 is substituted by proline (P) and the amino acids K68, S69, A70, R71 and S72 are deleted.

(SEQ ID NO: 33)
tlcgaelvdalqfvcgdrgfyfnkptgygsssrpapqtgivdeccfrscd
lrrlemycaplkpavraqrhtdmpktqkevhlknasrgsagnknyrm.

### Example 34

G1, P2, E3 are deleted, amino acid R36 and R37 are both substituted by glutamine (Q) and the amino acids K68, S69, A70, R71 and S72 are deleted.

(SEQ ID NO: 34)
tlcgaelvdalqfvcgdrgfyfnkptgygsssqqapqtgivdeccfrscd
lrrlemycaplkpaqvraqrhtdmpktqkevhlknasrgsagnknyrm.

### Example 35

G1, P2, E3 are deleted, amino acid R36 is substituted by glutamine (Q), R37 is substituted by alanine and the amino acids K68, S69, A70, R71 and S72 are deleted.

(SEQ ID NO: 35)
tlcgaelvdalqfvcgdrgfyfnkptgygsssqaapqtgivdeccfrscd
lrrlemycaplkpavraqrhtdmpktqkevhlknasrgsagnknyrm.

### Example 36

G1, P2, E3 are deleted, amino acid R36 is substituted by glutamine (Q) and the amino acids K68, S69, A70, R71 and S72 are deleted and amino acid R74 is mutated to glutamine (Q).

(SEQ ID NO: 36)
tlcgaelvdalqfvcgdrgfyfnkptgygsssqrapqtgivdeccfrscd
lrrlemycaplkpavqaqrhtdmpktqkevhlknasrgsagnknyrm.

### Example 37

G1, P2, E3 are deleted, amino acid R36 is substituted by glutamine (Q) and the amino acids K68, S69, A70, R71 and S72 are deleted and amino acids R74 and R77 are mutated to glutamine (Q).

(SEQ ID NO: 37)
tlcgaelvdalqfvcgdrgfyfnkptgygsssqrapqtgivdeccfrscd
lrrlemycaplkpavqaqqhtdmpktqkevhlknasrgsagnknyrm.

### Example 38

G1, P2, E3 are deleted, amino acid R36 is substituted by glutamine (Q) and the amino acids K68, S69, A70, R71 and S72 are deleted and amino acids R74, R77 and R104 are mutated to glutamine (Q).

(SEQ ID NO: 38)
tlcgaelvdalqfvcgdrgfyfnkptgygsssqrapqtgivdeccfrscd
lrrlemycaplkpavqaqqhtdmpktqkevhlknasrgsagnknyqm.

### Example 39

G1, P2, E3 are deleted, amino acid R37 is substituted by glutamic acid (E) and the amino acids K68, S69, A70, R71 and S72 are deleted and amino acid R74 is mutated to glutamine (Q).

(SEQ ID NO: 39)
tlcgaelvdalqfvcgdrgfyfnkptgygsssreapqtgivdeccfrscd
lrrlemycaplkpavqaqrhtdmpktqkevhlknasrgsagnknyrm.

### Example 40

G1, P2, E3 are deleted, amino acid R37 is substituted by glutamic acid (E) and the amino acids K68, S69, A70, R71 and S72 are deleted and amino acids R74 and R77 are mutated to glutamine (Q).

(SEQ ID NO: 40)
tlcgaelvdalqfvcgdrgfyfnkptgygsssreapqtgivdeccfrscd
lrrlemycaplkpavqaqqhtdmpktqkevhlknasrgsagnknyrm.

### Example 41

G1, P2, E3 are deleted, amino acid R37 is substituted by glutamic acid (E) and the amino acids K68, S69, A70, R71 and S72 are deleted and amino acids R74, R77 and R104 are mutated to glutamine (Q).

(SEQ ID NO: 41)
tlcgaelvdalqfvcgdrgfyfnkptgygsssreapqtgivdeccfrscd
lrrlemycaplkpavqaqqhtdmpktqkevhlknasrgsagnknyqm.

### Example 42

G1, P2, E3 are deleted, amino acid R37 is substituted by alanine (A) and the amino acids K68, S69, A70, R71 and S72 are deleted and amino acid R74 is mutated to glutamine (Q).

(SEQ ID NO: 42)
tlcgaelvdalqfvcgdrgfyfnkptgygsssraapqtgivdeccfrscd
lrrlemycaplkpavqaqrhtdmpktqkevhlknasrgsagnknyrm.

### Example 43

G1, P2, E3 are deleted, amino acid R37 is substituted by alanine (A) and the amino acids K68, S69, A70, R71 and S72 are deleted and amino acids R74 and R77 are mutated to glutamine (Q).

(SEQ ID NO: 43)
tlcgaelvdalqfvcgdrgfyfnkptgygsssraapqtgivdeccfrscd
lrrlemycaplkpavqaqqhtdmpktqkevhlknasrgsagnknyrm.

### Example 44

G1, P2, E3 are deleted, amino acid R37 is substituted by alanine (A) and the amino acids K68, S69, A70, R71 and S72 are deleted and amino acids R74, R77 and R104 are mutated to glutamine

(SEQ ID NO: 44)
tlcgaelvdalqfvcgdrgfyfnkptgygsssraapqtgivdeccfrscd
lrrlemycaplkpavqaqqhtdmpktqkevhlknasrgsagnknyqm.

### Example 45

G1, P2, E3 are deleted, amino acid R37 is substituted by proline (P) and the amino acids K68, S69, A70, R71 and S72 are deleted and amino acid R74 is mutated to glutamine (Q).

(SEQ ID NO: 45)
tlcgaelvdalqfvcgdrgfyfnkptgygsssrpapqtgivdeccfrscd
lrrlemycaplkpavqaqrhtdmpktqkevhlknasrgsagnknyrm.

### Example 46

G1, P2, E3 are deleted, amino acid R37 is substituted by proline (P) and the amino acids K68, S69, A70, R71 and S72 72 are deleted and amino acids R74 and R77 are mutated to glutamine (Q).

(SEQ ID NO: 46)
tlcgaelvdalqfvcgdrgfyfnkptgygsssrpapqtgivdeccfrscd
lrrlemycaplkpavqaqqhtdmpktqkevhlknasrgsagnknyrm.

### Example 47

G1, P2, E3 are deleted, amino acid R37 is substituted by proline (P) and the amino acids K68, S69, A70, R71 and S72 are deleted and amino acids R74, R77 and R104 are mutated to glutamine (Q).

(SEQ ID NO: 47)
tlcgaelvdalqfvcgdrgfyfnkptgygsssrpapqtgivdeccfrscd
lrrlemycaplkpavqaqqhtdmpktqkevhlknasrgsagnknyqm.

### Example 48

G1, P2, E3 are deleted, amino acid R36 and R37 are both substituted by glutamine (Q) and the amino acids K68, S69, A70, R71 and S72 are deleted and amino acid R74 is mutated to glutamine (Q).

(SEQ ID NO: 48)
tlcgaelvdalqfvcgdrgfyfnkptgygsssqqapqtgivdeccfrscd
lrrlemycaplkpavqaqrhtdmpktqkevhlknasrgsagnknyrm.

### Example 49

G1, P2, E3 are deleted, amino acid R36 and R37 are both substituted by glutamine (Q) and the amino acids K68, S69, A70, R71 and S72 are deleted and amino acids R74 and R77 are mutated to glutamine (Q).

(SEQ ID NO: 49)
tlcgaelvdalqfvcgdrgfyfnkptgygsssqqapqtgivdeccfrscd
lrrlemycaplkpavqaqqhtdmpktqkevhlknasrgsagnknyrm.

### Example 50

G1, P2, E3 are deleted, amino acid R36 is substituted by glutamine (Q), R37 is substituted by alanine and the amino acids K68, S69, A70, R71 and S72 are deleted and amino acids R74 and R77 are mutated to glutamine (Q).

(SEQ ID NO: 50)
tlcgaelvdalqfvcgdrgfyfnkptgygsssqaapqtgivdeccfrscd
lrrlemycaplkpavqaqqhtdmpktqkevhlknasrgsagnknyrm.

### Example 51

G1, P2, E3 are deleted, amino acid R36 and R37 are both substituted by glutamine (Q) and the amino acids K68, S69, A70, R71 and S72 are deleted and amino acids R74, R77 and R104 are mutated to glutamine (Q).

(SEQ ID NO: 51)
tlcgaelvdalqfvcgdrgfyfnkptgygsssqqapqtgivdeccfrscd
lrrlemycaplkpavqaqqhtdmpktqkevhlknasrgsagnknyqm.

### Example 52

G1, P2, E3 are deleted, amino acid R36 is substituted by glutamine (Q), R37 is substituted by alanine and the amino acids K68, S69, A70, R71 and S72 are deleted and amino acids R74, R77 and R104 are mutated to glutamine (Q).

(SEQ ID NO: 52)
tlcgaelvdalqfvcgdrgfyfnkptgygsssqaapqtgivdeccfrscd
lrrlemycaplkpavqaqqhtdmpktqkevhlknasrgsagnknyqm.

### Example 53

E3 is deleted, amino acid R36 is substituted by glutamine (Q) and the amino acids R71 and S72 are deleted.

(SEQ ID NO: 53)
gptlcgaelvdalqfvcgdrgfyfnkptgygsssqrapqtgivdeccfrs
cdlrrlemycaplkpaksavraqrhtdmpktqkevhlknasrgsagnkny
rm.

### Example 54

E3 is deleted, amino acid R37 is substituted by glutamic acid (E) and the amino acids R71 and S72 are deleted.

(SEQ ID NO: 54)
gptlcgaelvdalqfvcgdrgfyfnkptgygsssreapqtgivdeccfrs
cdlrrlemycaplkpaksavraqrhtdmpktqkevhlknasrgsagnkny
rm.

### Example 55

E3 is deleted, amino acid R37 is substituted by alanine and the amino acids R71 and S72 are deleted.

(SEQ ID NO: 55 (variant 63))
gptlcgaelvdalqfvcgdrgfyfnkptgygsssraapqtgivdeccfrs
cdlrrlemycaplkpaksavraqrhtdmpktqkevhlknasrgsagnkny
rm.

### Example 56

E3 is deleted, amino acid R37 is substituted by proline (P) and the amino acids R71 and S72 are deleted.

(SEQ ID NO: 56)
gptlcgaelvdalqfvcgdrgfyfnkptgygsssrpapqtgivdeccfrs
cdlrrlemycaplkpaksavraqrhtdmpktqkevhlknasrgsagnkny
rm.

### Example 57

E3 is deleted, amino acid R36 and R37 are both substituted by glutamine (Q) and the amino acids R71 and S72 are deleted.

(SEQ ID NO: 57)
gptlcgaelvdalqfvcgdrgfyfnkptgygsssqqapqtgivdeccfrs
cdlrrlemycaplkpaksavraqrhtdmpktqkevhlknasrgsagnkny
rm.

### Example 58

E3 is deleted, amino acid R36 is substituted by glutamine (Q), R37 is substituted by alanine and the amino acids R71 and S72 are deleted.

(SEQ ID NO: 58)
gptlcgaelvdalqfvcgdrgfyfnkptgygsssqaapqtgivdeccfrs
cdlrrlemycaplkpaksavraqrhtdmpktqkevhlknasrgsagnkny
rm.

### Example 59

E3 is deleted, amino acid R36 is substituted by glutamine (Q) and the amino acids R71 and S72 are deleted and amino acid R74 is mutated to glutamine (Q).

(SEQ ID NO: 59)
gptlcgaelvdalqfvcgdrgfyfnkptgygsssqrapqtgivdeccfrs
cdlrrlemycaplkpaksavqaqrhtdmpktqkevhlknasrgsagnkny
rm.

### Example 60

E3 is deleted, amino acid R36 is substituted by glutamine (Q) and the amino acids R71 and S72 are deleted and amino acids R74 and R77 are mutated to glutamine (Q).

(SEQ ID NO: 60)
gptlcgaelvdalqfvcgdrgfyfnkptgygsssqrapqtgivdeccfrs
cdlrrlemycaplkpaksavqaqqhtdmpktqkevhlknasrgsagnkny
rm.

### Example 61

E3 is deleted, amino acid R36 is substituted by glutamine (Q) and the amino acids R71 and S72 are deleted and amino acids R74, R77 and R104 are mutated to glutamine (Q).

(SEQ ID NO: 61)
gptlcgaelvdalqfvcgdrgfyfnkptgygsssqrapqtgivdeccfrs
cdlrrlemycaplkpaksavqaqqhtdmpktqkevhlknasrgsagnkny
qm.

### Example 62

E3 is deleted, amino acid R37 is substituted by glutamic acid (E) and the amino acids R71 and S72 are deleted and amino acid R77 is mutated to glutamine (Q).

(SEQ ID NO: 62)
gptlcgaelvdalqfvcgdrgfyfnkptgygsssreapqtgivdeccfrs
cdlrrlemycaplkpaksavraqqhtdmpktqkevhlknasrgsagnkny
rm.

### Example 63

E3 is deleted, amino acid R37 is substituted by glutamic acid (E) and the amino acids R71 and S72 are deleted and amino acids R74 and R77 are mutated to glutamine (Q).

(SEQ ID NO: 63)
gptlcgaelydalqfvcgdrgfyfnkptgygsssreapqtgiydeccfrs
cdlrrlemycaplkpaksavqaqqhtdmpktqkevhlknasrgsagnkny
rm.

### Example 64

E3 is deleted, amino acid R37 is substituted by glutamic acid (E) and the amino acids R71 and S72 are deleted and amino acids R74, R77 and R104 are mutated to glutamine (Q).

(SEQ ID NO: 64)
gptlcgaelvdalqfvcgdrgfyfnkptgygsssreapqtgivdeccfrs
cdlrrlemycaplkpaksavqaqqhtdmpktqkevhlknasrgsagnkny
qm.

### Example 65

E3 is deleted, amino acid R37 is substituted by alanine (A) and the amino acids R71 and S72 are deleted and amino acid R74 is mutated to glutamine (Q).

(SEQ ID NO: 65)
gptlcgaelvdalqfvcgdrgfyfnkptgygsssraapqtgivdeccfrs
cdlrrlemycaplkpaksavqaqrhtdmpktqkevhlknasrgsagnkny
rm.

### Example 66

E3 is deleted, amino acid R37 is substituted by alanine (A) and the amino acids R71 and S72 are deleted and amino acids R74 and R77 are mutated to glutamine (Q).

(SEQ ID NO: 66)
gptlcgaelvdalqfvcgdrgfyfnkptgygsssraapqtgiydeccfrs
cdlrrlemycaplkpaksavqaqqhtdmpktqkevhlknasrgsagnkny
rm.

### Example 67

E3 is deleted, amino acid R37 is substituted by alanine (A) and the amino acids R71 and S72 are deleted and amino acids R74, R77 and R104 are mutated to glutamine (Q).

(SEQ ID NO: 67)
gptlcgaelvdalqfvcgdrgfyfnkptgygsssraapqtgivdeccfrs
cdlrrlemycaplkpaksavqaqqhtdmpktqkevhlknasrgsagnkny
qm.

### Example 68

E3 is deleted, amino acid R37 is substituted by proline (P) and the amino acids R71 and S72 are deleted and amino acid R74 is mutated to glutamine (Q).

(SEQ ID NO: 68)
gptlcgaelvdalqfvcgdrgfyfnkptgygsssrpapqtgivdeccfrs
cdlrrlemycaplkpaksavqaqrhtdmpktqkevhlknasrgsagnkny
rm.

### Example 69

E3 is deleted, amino acid R37 is substituted by proline (P) and the amino acids R71 and S72 are deleted and amino acids R74 and R77 are mutated to glutamine (Q).

(SEQ ID NO: 69)
gptlcgaelvdalqfvcgdrgfyfnkptgygsssrpapqtgivdeccfrs
cdlrrlemycaplkpaksavqaqqhtdmpktqkevhlknasrgsagnkny
rm.

### Example 70

E3 is deleted, amino acid R37 is substituted by proline (P) and the amino acids R71 and S72 are deleted and amino acids R74, R77 and R104 are mutated to glutamine (Q).

(SEQ ID NO: 70)
gptlcgaelvdalqfvcgdrgfyfnkptgygsssrpapqtgivdeccfrs
cdlrrlemycaplkpaksavqaqqhtdmpktqkevhlknasrgsagnkny
qm.

### Example 71

E3 is deleted, amino acid R36 and R37 are both substituted by glutamine (Q) and the amino acids R71 and S72 are deleted and amino acid R74 is mutated to glutamine (Q).

(SEQ ID NO: 71)
gptlcgaelvdalqfvcgdrgfyfnkptgygsssqqapqtgivdeccfrs
cdlrrlemycaplkpaksavqaqrhtdmpktqkevhlknasrgsagnkny
rm.

### Example 72

E3 is deleted, amino acid R36 and R37 are both substituted by glutamine (Q) and the amino acids R71 and S72 are deleted and amino acids R74 and R77 are mutated to glutamine (Q).

(SEQ ID NO: 72)
gptlcgaelvdalqfvcgdrgfyfnkptgygsssqqapqtgivdeccfrs
cdlrrlemycaplkpaksavqaqqhtdmpktqkevhlknasrgsagnkny
rm.

### Example 73

E3 is deleted, amino acid R36 is substituted by glutamine (Q), R37 is substituted by alanine and the amino acids R71 and S72 are deleted and amino acids R74 and R77 are mutated to glutamine (Q).

(SEQ ID NO: 73)
gptlcgaelvdalqfvcgdrgfyfnkptgygsssqaapqtgivdeccfrs
cdlrrlemycaplkpaksavqaqqhtdmpktqkevhlknasrgsagnkny
rm.

### Example 74

E3 is deleted, amino acid R36 and R37 are both substituted by glutamine (Q) and the amino acids R71 and S72 are deleted and amino acids R74, R77 and R104 are mutated to glutamine (Q).

(SEQ ID NO: 74)
gptlcgaelvdalqfvcgdrgfyfnkptgygsssqqapqtgivdeccfrs
cdlrrlemycaplkpaksavqaqqhtdmpktqkevhlknasrgsagnkny
qm.

### Example 75

E3 is deleted, amino acid R36 is substituted by glutamine (Q), R37 is substituted by alanine and the amino acids R71 and S72 are deleted and amino acids R74, R77 and R104 are mutated to glutamine (Q).

(SEQ ID NO: 75)
gptlcgaelvdalqfvcgdrgfyfnkptgygsssqaapqtgivdeccfrs
cdlrrlemycaplkpaksavqaqqhtdmpktqkevhlknasrgsagnkny
qm.

### Example 76

E3 is deleted, amino acid R36 is substituted by glutamine (Q) and the amino acids K68, S69, A70, R71 and S72 are deleted.

(SEQ ID NO: 76)
gptlcgaelvdalqfvcgdrgfyfnkptgygsssqrapqtgivdeccfrs
cdlrrlemycaplkpavraqrhtdmpktqkevhlknasrgsagnknyrm.

### Example 77

E3 is deleted, amino acid R37 is substituted by glutamic acid (E) and the amino acids K68, S69, A70, R71 and S72 are deleted.

(SEQ ID NO: 77)
gptlcgaelvdalqfvcgdrgfyfnkptgygsssreapqtgivdeccfrs
cdlrrlemycaplkpavraqrhtdmpktqkevhlknasrgsagnknyrm.

### Example 78

E3 is deleted, amino acid R37 is substituted by alanine and the amino acids K68, S69, A70, R71 and S72 are deleted.

(SEQ ID NO: 78)
gptlcgaelvdalqfvcgdrgfyfnkptgygsssraapqtgivdeccfrs
cdlrrlemycaplkpavraqrhtdmpktqkevhlknasrgsagnknyrm.

### Example 79

E3 is deleted, amino acid R37 is substituted by proline (P) and the amino acids K68, S69, A70, R71 and S72 are deleted.

(SEQ ID NO: 79)
gptlcgaelvdalqfvcgdrgfyfnkptgygsssrpapqtgivdeccfrs
cdlrrlemycaplkpavraqrhtdmpktqkevhlknasrgsagnknyrm.

### Example 80

E3 is deleted, amino acid R36 and R37 are both substituted by glutamine (Q) and the amino acids K68, S69, A70, R71 and S72 are deleted.

(SEQ ID NO: 80)
gptlcgaelvdalqfvcgdrgfyfnkptgygsssqqapqtgivdeccfrs
cdlrrlemycaplkpaqvraqrhtdmpktqkevhlknasrgsagnknyrm.

### Example 81

E3 is deleted, amino acid R36 is substituted by glutamine (Q), R37 is substituted by alanine and the amino acids K68, S69, A70, R71 and S72 are deleted.

(SEQ ID NO: 81)
gptlcgaelvdalqfvcgdrgfyfnkptgygsssqaapqtgivdeccfrs
cdlrrlemycaplkpavraqrhtdmpktqkevhlknasrgsagnknyrm.

### Example 82

E3 is deleted, amino acid R36 is substituted by glutamine (Q) and the amino acids K68, S69, A70, R71 and S72 are deleted and amino acid R74 is mutated to glutamine (Q).

(SEQ ID NO: 82)
gptlcgaelvdalqfvcgdrgfyfnkptgygsssqrapqtgivdeccfrs
cdlrrlemycaplkpavqaqrhtdmpktqkevhlknasrgsagnknyrm.

### Example 83

E3 is deleted, amino acid R36 is substituted by glutamine (Q) and the amino acids K68, S69, A70, R71 and S72 are deleted and amino acids R74 and R77 are mutated to glutamine (Q).

(SEQ ID NO: 83)
gptlcgaelvdalqfvcgdrgfyfnkptgygsssqrapqtgivdeccfrs
cdlrrlemycaplkpavqaqqhtdmpktqkevhlknasrgsagnknyrm.

### Example 84

E3 is deleted, amino acid R36 is substituted by glutamine (Q) and the amino acids K68, S69, A70, R71 and S72 are deleted and amino acids R74, R77 and R104 are mutated to glutamine (Q).

(SEQ ID NO: 84)
gptlcgaelvdalqfvcgdrgfyfnkptgygsssqrapqtgivdeccfrs
cdlrrlemycaplkpavqaqqhtdmpktqkevhlknasrgsagnknyqm.

### Example 85

E3 is deleted, amino acid R37 is substituted by glutamic acid (E) and the amino acids K68, S69, A70, R71 and S72 are deleted and amino acid R74 is mutated to glutamine (Q).

(SEQ ID NO: 85)
gptlcgaelvdalqfvcgdrgfyfnkptgygsssreapqtgivdeccfrs
cdlrrlemycaplkpavqaqrhtdmpktqkevhlknasrgsagnknyrm.

### Example 86

E3 is deleted, amino acid R37 is substituted by glutamic acid (E) and the amino acids K68, S69, A70, R71 and S72 are deleted and amino acids R74 and R77 are mutated to glutamine (Q).

(SEQ ID NO: 86)
gptlcgaelvdalqfvcgdrgfyfnkptgygsssreapqtgivdeccfrs
cdlrrlemycaplkpavqaqqhtdmpktqkevhlknasrgsagnknyrm.

### Example 87

E3 is deleted, amino acid R37 is substituted by glutamic acid (E) and the amino acids K68, S69, A70, R71 and S72 are deleted and amino acids R74, R77 and R104 are mutated to glutamine (Q).

(SEQ ID NO: 87)
gptlcgaelvdalqfvcgdrgfyfnkptgygsssreapqtgivdeccfrs
cdlrrlemycaplkpavqaqqhtdmpktqkevhlknasrgsagnknyqm.

### Example 88

E3 is deleted, amino acid R37 is substituted by alanine (A) and the amino acids K68, S69, A70, R71 and S72 are deleted and amino acid R74 is mutated to glutamine (Q).

(SEQ ID NO: 88)
gptlcgaelvdalqfvcgdrgfyfnkptgygsssraapqtgivdeccfrs
cdlrrlemycaplkpavqaqrhtdmpktqkevhlknasrgsagnknyrm.

### Example 89

E3 is deleted, amino acid R37 is substituted by alanine (A) and the amino acids K68, S69, A70, R71 and S72 are deleted and amino acids R74 and R77 are mutated to glutamine (Q).

(SEQ ID NO: 89)
gptlcgaelvdalqfvcgdrgfyfnkptgygsssraapqtgivdeccfrs
cdlrrlemycaplkpavqaqqhtdmpktqkevhlknasrgsagnknyrm.

### Example 90

E3 is deleted, amino acid R37 is substituted by alanine (A) and the amino acids K68, S69, A70, R71 and S72 are deleted and amino acids R74, R77 and R104 are mutated to glutamine

(SEQ ID NO: 90)
gptlcgaelvdalqfvcgdrgfyfnkptgygsssraapqtgivdeccfrs
cdlrrlemycaplkpavqaqqhtdmpktqkevhlknasrgsagnknyqm.

### Example 91

E3 is deleted, amino acid R37 is substituted by proline (P) and the amino acids K68, S69, A70, R71 and S72 are deleted and amino acid R74 is mutated to glutamine (Q).

(SEQ ID NO: 91)
gptlcgaelvdalqfvcgdrgfyfnkptgygsssrpapqtgivdeccfrs
cdlrrlemycaplkpavqaqrhtdmpktqkevhlknasrgsagnknyrm.

### Example 92

E3 is deleted, amino acid R37 is substituted by proline (P) and the amino acids K68, S69, A70, R71 and S72 72 are deleted and amino acids R74 and R77 are mutated to glutamine (Q).

(SEQ ID NO: 92)
gptlcgaelvdalqfvcgdrgfyfnkptgygsssrpapqtgivdeccfrs
cdlrrlemycaplkpavqaqqhtdmpktqkevhlknasrgsagnknyrm.

### Example 93

E3 is deleted, amino acid R37 is substituted by proline (P) and the amino acids K68, S69, A70, R71 and S72 are deleted and amino acids R74, R77 and R104 are mutated to glutamine (Q).

(SEQ ID NO: 93)
gptlcgaelvdalqfvcgdrgfyfnkptgygsssrpapqtgivdeccfrs
cdlrrlemycaplkpavqaqqhtdmpktqkevhlknasrgsagnknyqm.

### Example 94

E3 is deleted, amino acid R36 and R37 are both substituted by glutamine (Q) and the amino acids K68, S69, A70, R71 and S72 are deleted and amino acid R74 is mutated to glutamine (Q).

(SEQ ID NO: 94)
gptlcgaelvdalqfvcgdrgfyfnkptgygsssqqapqtgivdeccfrs
cdlrrlemycaplkpavqaqrhtdmpktqkevhlknasrgsagnknyrm.

### Example 95

E3 is deleted, amino acid R36 and R37 are both substituted by glutamine (Q) and the amino acids K68, S69, A70, R71 and S72 are deleted and amino acids R74 and R77 are mutated to glutamine (Q).

(SEQ ID NO: 95)
gptlcgaelvdalqfvcgdrgfyfnkptgygsssqqapqtgivdeccfrs
cdlrrlemycaplkpavqaqqhtdmpktqkevhlknasrgsagnknyrm.

### Example 96

E3 is deleted, amino acid R36 is substituted by glutamine (Q), R37 is substituted by alanine and the amino acids K68, S69, A70, R71 and S72 are deleted and amino acids R74 and R77 are mutated to glutamine (Q).

(SEQ ID NO: 96)
gptlcgaelvdalqfvcgdrgfyfnkptgygsssqaapqtgivdeccfrs
cdlrrlemycaplkpavqaqqhtdmpktqkevhlknasrgsagnknyrm.

### Example 97

E3 is deleted, amino acid R36 and R37 are both substituted by glutamine (Q) and the amino acids K68, S69, A70, R71 and S72 are deleted and amino acids R74, R77 and R104 are mutated to glutamine (Q).

(SEQ ID NO: 97)
gptlcgaelvdalqfvcgdrgfyfnkptgygsssqqapqtgivdeccfrs
cdlrrlemycaplkpavqaqqhtdmpktqkevhlknasrgsagnknyqm.

### Example 98

E3 is deleted, amino acid R36 is substituted by glutamine (Q), R37 is substituted by alanine and the amino acids K68, S69, A70, R71 and S72 are deleted and amino acids R74, R77 and R104 are mutated to glutamine (Q).

(SEQ ID NO: 98)
gptlcgaelvdalqfvcgdrgfyfnkptgygsssqaapqtgivdeccfrs
cdlrrlemycaplkpavqaqqhtdmpktqkevhlknasrgsagnknyqm.

1. A process for preparing a composition of a pegylated therapeutic protein, wherein in said composition at least 61 percent of the mono pegylated therapeutic protein fraction comprised in said composition is N-terminally mono-pegylated therapeutic protein, comprising the step of:
reacting in an aqueous medium a therapeutic protein with a water-soluble polyethylene glycol under conditions of a reductive alkylation, characterized in that the reaction is performed within a pH range of about 6.5 to 7.5, to provide the composition of the pegylated therapeutic protein.

2. A process for preparing a composition according to claim 1, wherein the therapeutic protein is a human IGF-1 precursor protein or a variant thereof.

3. The process according to claim 2, wherein the IGF1 precursor protein is a human IGF-1Ea peptide precursor protein wherein amino acid E3 is deleted, amino acid R37 is substituted by alanine and the amino acids R71 and S72 are deleted, and wherein the numbering of the amino acids corresponds to SEQ ID NO: 5.

4. The process according to claim 2, wherein the IGF1 precursor protein is a human IGF-1Ea peptide precursor protein consisting of SEQ ID NO: 55.

5. A process according to claim 1, characterized in that the pegylation reaction is performed in the presence of α-cyclodextrine (α-CD).

6. A process according to claim 1, characterized in that the PEG has an overall molecular weight of from 20 to 100 kDa.

7. A process according to claim 6, characterized in that the PEG has an overall molecular weight of about 30 kDa.

8. A process for preparing a composition of a mono pegylated therapeutic protein, wherein in said composition at least 65 percent of the mono pegylated protein fraction comprised in said composition is N-terminally mono-pegylated therapeutic protein, comprising the steps of:
(a) reacting in an aqueous medium the therapeutic proteins with a water-soluble polyethylene glycol under conditions of a reductive alkylation, characterized in that the coupling reaction is performed within a pH range of about 6.5 to 7.5,
(b) performing a chromatography step with the compositions obtained in step (a), to provide the composition of the mono pegylated therapeutic protein.

9. A process for preparing a composition of according to claim 8, wherein the therapeutic protein is a human IGF-1 precursor protein or a variant thereof.

10. The process according to claim 9, wherein the IGF1 precursor protein is a human IGF-1Ea peptide precursor protein wherein amino acid E3 is deleted, amino acid R37 is substituted by alanine and the amino acids R71 and S72 are deleted, and wherein the numbering of the amino acids corresponds to SEQ ID NO: 5.

11. The process according to claim 9, wherein the IGF1 precursor protein is a human IGF-1Ea peptide precursor protein consisting of SEQ ID NO: 55.

12. A process according to claim 8, characterized in that the pegylation reaction is performed in the presence of α-cyclodextrine (α-CD).

13. A process according to claim 8, characterized in that the PEG has an overall molecular weight of from 20 to 100 kDa.

14. A process according to claim 13, characterized in that the PEG has an overall molecular weight of about 30 kDa.

15. The process according to claim 8, wherein the chromatography step is a cation exchange chromatography (CEX), comprising pooling those fractions containing the N-terminally mono-pegylated therapeutic protein, to provide the composition of the mono pegylated therapeutic protein.

16. A process according to claim 8, further comprising the additional steps of
b) Ultrafiltration (UF)/diafiltration (DF) concentration and buffer exchange, and
c) final filtration.

17. A composition produced according to the process of claim 1.

18. A composition produced according to the process of claim 8.

19. A composition produced according to the process of claim 8, wherein in said composition at least 70% of the pegylated IGF-1 precursor protein fraction comprised in said composition is a mixture of N-terminally and lysine residue mono-pegylated IGF-1 precursor protein.

20. A composition produced according to the process of claim 1, wherein the IGF1 precursor protein is a human IGF-1Ea peptide precursor protein wherein amino acid E3 is deleted, amino acid R37 is substituted by alanine and the amino acids R71 and S72 are deleted, and wherein the numbering of the amino acids corresponds to SEQ ID NO: 5.

21. A composition produced according to the process of claim 1, wherein the IGF1 precursor protein is a human IGF-1Ea peptide precursor protein comprising the amino acid sequence of SEQ ID NO: 55.

22. A composition produced according to the process of claim 8, wherein the IGF1 precursor protein is a human IGF-1Ea peptide precursor protein wherein amino acid E3 is deleted, amino acid R37 is substituted by alanine and the amino acids R71 and S72 are deleted, and wherein the numbering of the amino acids corresponds to SEQ ID NO: 5.

23. A composition produced according to the process of claim 8, wherein the IGF1 precursor protein is a human IGF-1Ea peptide precursor protein comprising the amino acid sequence of SEQ ID NO: 55.

24. A composition according to claim 17, in a pharmaceutically acceptable form for use as a medicament.

25. The composition of claim 24 for use in the treatment of a muscle disorder in a patient in need thereof.

26. The composition of claim 24 for use in the treatment of burn patients suffering from loss of lean body mass and/or muscle wasting and/or muscle atrophy.

27. The composition of claim 24 for use in the treatment of muscle atrophy selected from the group consisting of obesity-associated sarcopenia, sarcopenia, and diabetes-associated muscle atrophy.

28. The composition of claim 24 for use in the treatment of chronic obstructive pulmonary disease (COPD) patients.

29. The composition of claim 24 for use in the treatment of Spinal and Bulbar Muscular Atrophy (SBMA or Kennedy disease) or chronic kidney disease patients.

30. A composition according to claim 18, in a pharmaceutically acceptable form for use as a medicament.

31. The composition of claim 30 for use in the treatment of a muscle disorder in a patient in need thereof.

32. The composition of claim 30 for use in the treatment of burn patients suffering from loss of lean body mass and/or muscle wasting and/or muscle atrophy.

33. The composition of claim 30 for use in the treatment of muscle atrophy selected from the group consisting of obesity-associated sarcopenia, sarcopenia, and diabetes-associated muscle atrophy.

34. The composition of claim 30 for use in the treatment of chronic obstructive pulmonary disease (COPD) patients.

35. The composition of claim 30 for use in the treatment of Spinal and Bulbar Muscular Atrophy (SBMA or Kennedy disease) or chronic kidney disease patients.

36. A method of treating a muscle disorder in a patient in need thereof, the method comprising administering a therapeutically effective amount of the composition of claim 17 to a subject.

37. A method according to claim 36, wherein the muscle disorder is a muscle atrophy selected from the group consisting of obesity-associated sarcopenia, sarcopenia, and diabetes-associated muscle atrophy.

38. A method of treating burn patients suffering from loss of lean body mass and/or muscle wasting, the method comprising administering a therapeutically effective amount of the composition of claim 17 to a subject.

39. A method of treating chronic obstructive pulmonary disease (COPD) patients, the method comprising administering a therapeutically effective amount of the composition of claim 17 to a subject.

40. A method of treating Spinal and Bulbar Muscular Atrophy (SBMA or Kennedy disease) patients, the method comprising administering a therapeutically effective amount of the composition of claim 17 to a subject.

41. A method of treating chronic kidney disease patients, the method comprising administering a therapeutically effective amount of the composition of claim 17 to a subject.

42. A method of treating a muscle disorder in a patient in need thereof, the method comprising administering a therapeutically effective amount of the composition of claim 18 to a subject.

43. A method according to claim 42, wherein the muscle disorder is a muscle atrophy selected from the group consisting of obesity-associated sarcopenia, sarcopenia, and diabetes-associated muscle atrophy.

44. A method of treating burn patients suffering from loss of lean body mass and/or muscle wasting, the method comprising administering a therapeutically effective amount of the composition of claim 18 to a subject.

45. A method of treating chronic obstructive pulmonary disease (COPD) patients, the method comprising administering a therapeutically effective amount of the composition of claim 18 to a subject.

46. A method of treating Spinal and Bulbar Muscular Atrophy (SBMA or Kennedy disease) patients, the method comprising administering a therapeutically effective amount of the composition of claim 18 to a subject.

47. A method of treating chronic kidney disease patients, the method comprising administering a therapeutically effective amount of the composition of claim 18 to a subject.


SEQUENCE LISTING (101 sequences)

SEQ ID NO: 1 - protein, 70 aa, Homo sapiens
GPETLCGAELVDALQFVCGDRGFYFNKPTGYGSSSRRAPQTGIVDECCFRSCDLRRLEMYCAPLKPAKSA

SEQ ID NO: 2 - protein, 35 aa, Homo sapiens
RSVRAQRHTDMPKTQKEVHLKNASRGSAGNKNYRM

SEQ ID NO: 3 - protein, 77 aa, Homo sapiens
RSVRAQRHTDMPKTQKYQPPSTNKNTKSQRRKGWPKTHPGGEQKEGTEASLQIRGKKKEQRREIGSRNAECRGKKGK

SEQ ID NO: 4 - protein, 40 aa, Homo sapiens
RSVRAQRHTDMPKTQKYQPPSTNKNTKSQRRKGSTFEERK

SEQ ID NO: 5 - protein, 105 aa, Homo sapiens
GPETLCGAELVDALQFVCGDRGFYFNKPTGYGSSSRRAPQTGIVDECCFRSCDLRRLEMYCAPLKPAKSARSVRAQRHTDMPKTQKEVHLKNASRGSAGNKNYRM

SEQ ID NO: 6 - protein, 100 aa, Artificial Sequence
  Description of Artificial Sequence Synthetic Mutated human IGF-1 Protein
TLCGAELVDALQFVCGDRGFYFNKPTGYGSSSQRAPQTGIVDECCFRSCDLRRLEMYCAPLKPAKSAVRAQRHTDMPKTQKEVHLKNASRGSAGNKNYRM

SEQ ID NO: 7 - protein, 100 aa, Artificial Sequence
  Description of Artificial Sequence Synthetic Mutated human IGF-1 Protein
TLCGAELVDALQFVCGDRGFYFNKPTGYGSSSREAPQTGIVDECCFRSCDLRRLEMYCAPLKPAKSAVRAQRHTDMPKTQKEVHLKNASRGSAGNKNYRM

SEQ ID NO: 8 - protein, 100 aa, Artificial Sequence
  Description of Artificial Sequence Synthetic Mutated human IGF-1 Protein
TLCGAELVDALQFVCGDRGFYFNKPTGYGSSSRAAPQTGIVDECCFRSCDLRRLEMYCAPLKPAKSAVRAQRHTDMPKTQKEVHLKNASRGSAGNKNYRM

SEQ ID NO: 9 - protein, 100 aa, Artificial Sequence
  Description of Artificial Sequence Synthetic Mutated human IGF-1 Protein
TLCGAELVDALQFVCGDRGFYFNKPTGYGSSSRPAPQTGIVDECCFRSCDLRRLEMYCAPLKPAKSAVRAQRHTDMPKTQKEVHLKNASRGSAGNKNYRM

SEQ ID NO: 10 - protein, 100 aa, Artificial Sequence
  Description of Artificial Sequence Synthetic Mutated human IGF-1 Protein
TLCGAELVDALQFVCGDRGFYFNKPTGYGSSSQQAPQTGIVDECCFRSCDLRRLEMYCAPLKPAKSAVRAQRHTDMPKTQKEVHLKNASRGSAGNKNYRM

SEQ ID NO: 11 - protein, 100 aa, Artificial Sequence
  Description of Artificial Sequence Synthetic Mutated human IGF-1 Protein
TLCGAELVDALQFVCGDRGFYFNKPTGYGSSSQAAPQTSIVDECCFRSCDLRRLEMYCAPLKPAKSAVRAQRHTDMPKTQKEVHLKNASRGSAGNKNYRM

SEQ ID NO: 12 - protein, 100 aa, Artificial Sequence
  Description of Artificial Sequence Synthetic Mutated human IGF-1 Protein
TLCGAELVDALQFVCGDRGFYFNKPTGYGSSSQRAPQTSIVDECCFRSCDLRRLEMYCAPLKPAKSAVQAQRHTDMPKTQKEVHLKNASRGSAGNKNYRM

SEQ ID NO: 13 - protein, 100 aa, Artificial Sequence
  Description of Artificial Sequence Synthetic Mutated human IGF-1 Protein
TLCGAELVDALQFVCGDRGFYFNKPTGYGSSSQRAPQTSIVDECCFRSCDLRRLEMYCAPLKPAKSAVQAQQHTDMPKTQKEVHLKNASRGSAGNKNYRM

SEQ ID NO: 14 - protein, 100 aa, Artificial Sequence
  Description of Artificial Sequence Synthetic Mutated human IGF-1 Protein
TLCGAELVDALQFVCGDRGFYFNKPTGYGSSSQRAPQTSIVDECCFRSCDLRRLEMYCAPLKPAKSAVQAQQHTDMPKTQKEVHLKNASRGSAGNKNYQM

SEQ ID NO: 15 - protein, 100 aa, Artificial Sequence
  Description of Artificial Sequence Synthetic Mutated human IGF-1 Protein
TLCGAELVDALQFVCGDRGFYFNKPTGYGSSSREAPQTSIVDECCFRSCDLRRLEMYCAPLKPAKSAVRAQQHTDMPKTQKEVHLKNASRGSAGNKNYRM

SEQ ID NO: 16 - protein, 100 aa, Artificial Sequence
  Description of Artificial Sequence Synthetic Mutated human IGF-1 Protein
TLCGAELVDALQFVCGDRGFYFNKPTGYGSSSREAPQTSIVDECCFRSCDLRRLEMYCAPLKPAKSAVQAQQHTDMPKTQKEVHLKNASRGSAGNKNYRM

SEQ ID NO: 17 - protein, 100 aa, Artificial Sequence
  Description of Artificial Sequence Synthetic Mutated human IGF-1 Protein
TLCGAELVDALQFVCGDRGFYFNKPTGYGSSSREAPQTSIVDECCFRSCDLRRLEMYCAPLKPAKSAVQAQQHTDMPKTQKEVHLKNASRGSAGNKNYQM

SEQ ID NO: 18 - protein, 100 aa, Artificial Sequence; identical to SEQ ID NO: 8
  Description of Artificial Sequence Synthetic Mutated human IGF-1 Protein

SEQ ID NO: 19 - protein, 100 aa, Artificial Sequence
  Description of Artificial Sequence Synthetic Mutated human IGF-1 Protein
TLCGAELVDALQFVCGDRGFYFNKPTGYGSSSRAAPQTGIVDECCFRSCDLRRLEMYCAPLKPAKSAVQAQRHTDMPKTQKEVHLKNASRGSAGNKNYRM

SEQ ID NO: 20 - protein, 100 aa, Artificial Sequence
  Description of Artificial Sequence Synthetic Mutated human IGF-1 Protein
TLCGAELVDALQFVCGDRGFYFNKPTGYGSSSRAAPQTGIVDECCFRSCDLRRLEMYCAPLKPAKSAVQAQQHTDMPKTQKEVHLKNASRGSAGNKNYRM

SEQ ID NO: 21 - protein, 100 aa, Artificial Sequence
  Description of Artificial Sequence Synthetic Mutated human IGF-1 Protein
TLCGAELVDALQFVCGDRGFYFNKPTGYGSSSRAAPQTGIVDECCFRSCDLRRLEMYCAPLKPAKSAVQAQQHTDMPKTQKEVHLKNASRGSAGNKNYQM

SEQ ID NO: 22 - protein, 100 aa, Artificial Sequence
  Description of Artificial Sequence Synthetic Mutated human IGF-1 Protein
TLCGAELVDALQFVCGDRGFYFNKPTGYGSSSRPAPQTGIVDECCFRSCDLRRLEMYCAPLKPAKSAVQAQRHTDMPKTQKEVHLKNASRGSAGNKNYRM

SEQ ID NO: 23 - protein, 100 aa, Artificial Sequence
  Description of Artificial Sequence Synthetic Mutated human IGF-1 Protein
TLCGAELVDALQFVCGDRGFYFNKPTGYGSSSRPAPQTGIVDECCFRSCDLRRLEMYCAPLKPAKSAVQAQQHTDMPKTQKEVHLKNASRGSAGNKNYRM

SEQ ID NO: 24 - protein, 100 aa, Artificial Sequence
  Description of Artificial Sequence Synthetic Mutated human IGF-1 Protein
TLCGAELVDALQFVCGDRGFYFNKPTGYGSSSRPAPQTGIVDECCFRSCDLRRLEMYCAPLKPAKSAVQAQQHTDMPKTQKEVHLKNASRGSAGNKNYQM

SEQ ID NO: 25 - protein, 100 aa, Artificial Sequence
  Description of Artificial Sequence Synthetic Mutated human IGF-1 Protein
TLCGAELVDALQFVCGDRGFYFNKPTGYGSSSQQAPQTGIVDECCFRSCDLRRLEMYCAPLKPAKSAVQAQRHTDMPKTQKEVHLKNASRGSAGNKNYRM

SEQ ID NO: 26 - protein, 100 aa, Artificial Sequence
  Description of Artificial Sequence Synthetic Mutated human IGF-1 E Protein
TLCGAELVDALQFVCGDRGFYFNKPTGYGSSSQQAPQTGIVDECCFRSCDLRRLEMYCAPLKPAKSAVQAQQHTDMPKTQKEVHLKNASRGSAGNKNYRM

SEQ ID NO: 27 - protein, 100 aa, Artificial Sequence
  Description of Artificial Sequence Synthetic Mutated human IGF-1 Protein
TLCGAELVDALQFVCGDRGFYFNKPTGYGSSSQAAPQTGIVDECCFRSCDLRRLEMYCAPLKPAKSAVQAQQHTDMPKTQKEVHLKNASRGSAGNKNYRM

SEQ ID NO: 28 - protein, 100 aa, Artificial Sequence
  Description of Artificial Sequence Synthetic Mutated human IGF-1 Protein
TLCGAELVDALQFVCGDRGFYFNKPTGYGSSSQQAPQTGIVDECCFRSCDLRRLEMYCAPLKPAKSAVQAQQHTDMPKTQKEVHLKNASRGSAGNKNYQM

SEQ ID NO: 29 - protein, 100 aa, Artificial Sequence
  Description of Artificial Sequence Synthetic Mutated human IGF-1 Protein
TLCGAELVDALQFVCGDRGFYFNKPTGYGSSSQAAPQTGIVDECCFRSCDLRRLEMYCAPLKPAKSAVQAQQHTDMPKTQKEVHLKNASRGSAGNKNYQM

SEQ ID NO: 30 - protein, 97 aa, Artificial Sequence
  Description of Artificial Sequence Synthetic Mutated human IGF-1 Protein
TLCGAELVDALQFVCGDRGFYFNKPTGYGSSSQRAPQTGIVDECCFRSCDLRRLEMYCAPLKPAVRAQRHTDMPKTQKEVHLKNASRGSAGNKNYRM

SEQ ID NO: 31 - protein, 97 aa, Artificial Sequence
  Description of Artificial Sequence Synthetic Mutated human IGF-1 Protein
TLCGAELVDALQFVCGDRGFYFNKPTGYGSSSREAPQTGIVDECCFRSCDLRRLEMYCAPLKPAVRAQRHTDMPKTQKEVHLKNASRGSAGNKNYRM

SEQ ID NO: 32 - protein, 97 aa, Artificial Sequence
  Description of Artificial Sequence Synthetic Mutated human IGF-1 Protein
TLCGAELVDALQFVCGDRGFYFNKPTGYGSSSRAAPQTGIVDECCFRSCDLRRLEMYCAPLKPAVRAQRHTDMPKTQKEVHLKNASRGSAGNKNYRM

SEQ ID NO: 33 - protein, 97 aa, Artificial Sequence
  Description of Artificial Sequence Synthetic Mutated human IGF-1 Protein
TLCGAELVDALQFVCGDRGFYFNKPTGYGSSSRPAPQTGIVDECCFRSCDLRRLEMYCAPLKPAVRAQRHTDMPKTQKEVHLKNASRGSAGNKNYRM

SEQ ID NO: 34 - protein, 98 aa, Artificial Sequence
  Description of Artificial Sequence Synthetic Mutated human IGF-1 Protein
TLCGAELVDALQFVCGDRGFYFNKPTGYGSSSQQAPQTGIVDECCFRSCDLRRLEMYCAPLKPAQVRAQRHTDMPKTQKEVHLKNASRGSAGNKNYRM

SEQ ID NO: 35 - protein, 97 aa, Artificial Sequence
  Description of Artificial Sequence Synthetic Mutated human IGF-1 Protein
TLCGAELVDALQFVCGDRGFYFNKPTGYGSSSQAAPQTGIVDECCFRSCDLRRLEMYCAPLKPAVRAQRHTDMPKTQKEVHLKNASRGSAGNKNYRM

SEQ ID NO: 36 - protein, 97 aa, Artificial Sequence
  Description of Artificial Sequence Synthetic Mutated human IGF-1 Protein
TLCGAELVDALQFVCGDRGFYFNKPTGYGSSSQRAPQTGIVDECCFRSCDLRRLEMYCAPLKPAVQAQRHTDMPKTQKEVHLKNASRGSAGNKNYRM

SEQ ID NO: 37 - protein, 97 aa, Artificial Sequence
  Description of Artificial Sequence Synthetic Mutated human IGF-1 Protein
TLCGAELVDALQFVCGDRGFYFNKPTGYGSSSQRAPQTGIVDECCFRSCDLRRLEMYCAPLKPAVQAQQHTDMPKTQKEVHLKNASRGSAGNKNYRM

SEQ ID NO: 38 - protein, 97 aa, Artificial Sequence
  Description of Artificial Sequence Synthetic Mutated human IGF-1 Protein
TLCGAELVDALQFVCGDRGFYFNKPTGYGSSSQRAPQTGIVDECCFRSCDLRRLEMYCAPLKPAVQAQQHTDMPKTQKEVHLKNASRGSAGNKNYQM

SEQ ID NO: 39 - protein, 97 aa, Artificial Sequence
  Description of Artificial Sequence Synthetic Mutated human IGF-1 Protein
TLCGAELVDALQFVCGDRGFYFNKPTGYGSSSREAPQTGIVDECCFRSCDLRRLEMYCAPLKPAVQAQRHTDMPKTQKEVHLKNASRGSAGNKNYRM

SEQ ID NO: 40 - protein, 97 aa, Artificial Sequence
  Description of Artificial Sequence Synthetic Mutated human IGF-1 Protein
TLCGAELVDALQFVCGDRGFYFNKPTGYGSSSREAPQTGIVDECCFRSCDLRRLEMYCAPLKPAVQAQQHTDMPKTQKEVHLKNASRGSAGNKNYRM

SEQ ID NO: 41 - protein, 97 aa, Artificial Sequence
  Description of Artificial Sequence Synthetic Mutated human IGF-1 Protein
TLCGAELVDALQFVCGDRGFYFNKPTGYGSSSREAPQTGIVDECCFRSCDLRRLEMYCAPLKPAVQAQQHTDMPKTQKEVHLKNASRGSAGNKNYQM

SEQ ID NO: 42 - protein, 97 aa, Artificial Sequence
  Description of Artificial Sequence Synthetic Mutated human IGF-1 Protein
TLCGAELVDALQFVCGDRGFYFNKPTGYGSSSRAAPQTGIVDECCFRSCDLRRLEMYCAPLKPAVQAQRHTDMPKTQKEVHLKNASRGSAGNKNYRM

SEQ ID NO: 43 - protein, 97 aa, Artificial Sequence
  Description of Artificial Sequence Synthetic Mutated human IGF-1 Protein
TLCGAELVDALQFVCGDRGFYFNKPTGYGSSSRAAPQTGIVDECCFRSCDLRRLEMYCAPLKPAVQAQQHTDMPKTQKEVHLKNASRGSAGNKNYRM

SEQ ID NO: 44 - protein, 97 aa, Artificial Sequence
  Description of Artificial Sequence Synthetic Mutated human IGF-1 Protein
TLCGAELVDALQFVCGDRGFYFNKPTGYGSSSRAAPQTGIVDECCFRSCDLRRLEMYCAPLKPAVQAQQHTDMPKTQKEVHLKNASRGSAGNKNYQM

SEQ ID NO: 45 - protein, 97 aa, Artificial Sequence
  Description of Artificial Sequence Synthetic Mutated human IGF-1 Protein
TLCGAELVDALQFVCGDRGFYFNKPTGYGSSSRPAPQTGIVDECCFRSCDLRRLEMYCAPLKPAVQAQRHTDMPKTQKEVHLKNASRGSAGNKNYRM

SEQ ID NO: 46 - protein, 97 aa, Artificial Sequence
  Description of Artificial Sequence Synthetic Mutated human IGF-1 Protein
TLCGAELVDALQFVCGDRGFYFNKPTGYGSSSRPAPQTGIVDECCFRSCDLRRLEMYCAPLKPAVQAQQHTDMPKTQKEVHLKNASRGSAGNKNYRM

SEQ ID NO: 47 - protein, 97 aa, Artificial Sequence
  Description of Artificial Sequence Synthetic Mutated human IGF-1 Protein
TLCGAELVDALQFVCGDRGFYFNKPTGYGSSSRPAPQTGIVDECCFRSCDLRRLEMYCAPLKPAVQAQQHTDMPKTQKEVHLKNASRGSAGNKNYQM

SEQ ID NO: 48 - protein, 97 aa, Artificial Sequence
  Description of Artificial Sequence Synthetic Mutated human IGF-1 Protein
TLCGAELVDALQFVCGDRGFYFNKPTGYGSSSQQAPQTGIVDECCFRSCDLRRLEMYCAPLKPAVQAQRHTDMPKTQKEVHLKNASRGSAGNKNYRM

SEQ ID NO: 49 - protein, 97 aa, Artificial Sequence
  Description of Artificial Sequence Synthetic Mutated human IGF-1 Protein
TLCGAELVDALQFVCGDRGFYFNKPTGYGSSSQQAPQTGIVDECCFRSCDLRRLEMYCAPLKPAVQAQQHTDMPKTQKEVHLKNASRGSAGNKNYRM

SEQ ID NO: 50 - protein, 97 aa, Artificial Sequence
  Description of Artificial Sequence Synthetic Mutated human IGF-1 Protein
TLCGAELVDALQFVCGDRGFYFNKPTGYGSSSQAAPQTGIVDECCFRSCDLRRLEMYCAPLKPAVQAQQHTDMPKTQKEVHLKNASRGSAGNKNYRM

SEQ ID NO: 51 - protein, 97 aa, Artificial Sequence
  Description of Artificial Sequence Synthetic Mutated human IGF-1 Protein
TLCGAELVDALQFVCGDRGFYFNKPTGYGSSSQQAPQTGIVDECCFRSCDLRRLEMYCAPLKPAVQAQQHTDMPKTQKEVHLKNASRGSAGNKNYQM

SEQ ID NO: 52 - protein, 97 aa, Artificial Sequence
  Description of Artificial Sequence Synthetic Mutated human IGF-1 Protein
TLCGAELVDALQFVCGDRGFYFNKPTGYGSSSQAAPQTGIVDECCFRSCDLRRLEMYCAPLKPAVQAQQHTDMPKTQKEVHLKNASRGSAGNKNYQM

SEQ ID NO: 53 - protein, 102 aa, Artificial Sequence
  Description of Artificial Sequence Synthetic Mutated human IGF-1 Protein
GPTLCGAELVDALQFVCGDRGFYFNKPTGYGSSSQRAPQTGIVDECCFRSCDLRRLEMYCAPLKPAKSAVRAQRHTDMPKTQKEVHLKNASRGSAGNKNYRM

SEQ ID NO: 54 - protein, 102 aa, Artificial Sequence
  Description of Artificial Sequence Synthetic Mutated human IGF-1 Protein
GPTLCGAELVDALQFVCGDRGFYFNKPTGYGSSSREAPQTGIVDECCFRSCDLRRLEMYCAPLKPAKSAVRAQRHTDMPKTQKEVHLKNASRGSAGNKNYRM

SEQ ID NO: 55 - protein, 102 aa, Artificial Sequence
  Description of Artificial Sequence Synthetic Mutated human IGF-1 Protein
GPTLCGAELVDALQFVCGDRGFYFNKPTGYGSSSRAAPQTGIVDECCFRSCDLRRLEMYCAPLKPAKSAVRAQRHTDMPKTQKEVHLKNASRGSAGNKNYRM

SEQ ID NO: 56 - protein, 102 aa, Artificial Sequence
  Description of Artificial Sequence Synthetic Mutated human IGF-1 Protein
GPTLCGAELVDALQFVCGDRGFYFNKPTGYGSSSRPAPQTGIVDECCFRSCDLRRLEMYCAPLKPAKSAVRAQRHTDMPKTQKEVHLKNASRGSAGNKNYRM

SEQ ID NO: 57 - protein, 102 aa, Artificial Sequence
  Description of Artificial Sequence Synthetic Mutated human IGF-1 Protein
GPTLCGAELVDALQFVCGDRGFYFNKPTGYGSSSQQAPQTGIVDECCFRSCDLRRLEMYCAPLKPAKSAVRAQRHTDMPKTQKEVHLKNASRGSAGNKNYRM

SEQ ID NO: 58 - protein, 102 aa, Artificial Sequence
  Description of Artificial Sequence Synthetic Mutated human IGF-1 Protein
GPTLCGAELVDALQFVCGDRGFYFNKPTGYGSSSQAAPQTGIVDECCFRSCDLRRLEMYCAPLKPAKSAVRAQRHTDMPKTQKEVHLKNASRGSAGNKNYRM

SEQ ID NO: 59 - protein, 102 aa, Artificial Sequence
  Description of Artificial Sequence Synthetic Mutated human IGF-1 Protein
GPTLCGAELVDALQFVCGDRGFYFNKPTGYGSSSQRAPQTGIVDECCFRSCDLRRLEMYCAPLKPAKSAVQAQRHTDMPKTQKEVHLKNASRGSAGNKNYRM

SEQ ID NO: 60 - protein, 102 aa, Artificial Sequence
  Description of Artificial Sequence Synthetic Mutated human IGF-1 Protein
GPTLCGAELVDALQFVCGDRGFYFNKPTGYGSSSQRAPQTGIVDECCFRSCDLRRLEMYCAPLKPAKSAVQAQQHTDMPKTQKEVHLKNASRGSAGNKNYRM

SEQ ID NO: 61 - protein, 102 aa, Artificial Sequence
  Description of Artificial Sequence Synthetic Mutated human IGF-1 Protein
GPTLCGAELVDALQFVCGDRGFYFNKPTGYGSSSQRAPQTGIVDECCFRSCDLRRLEMYCAPLKPAKSAVQAQQHTDMPKTQKEVHLKNASRGSAGNKNYQM

SEQ ID NO: 62 - protein, 102 aa, Artificial Sequence
  Description of Artificial Sequence Synthetic Mutated human IGF-1 Protein
GPTLCGAELVDALQFVCGDRGFYFNKPTGYGSSSREAPQTGIVDECCFRSCDLRRLEMYCAPLKPAKSAVRAQQHTDMPKTQKEVHLKNASRGSAGNKNYRM

SEQ ID NO: 63 - protein, 102 aa, Artificial Sequence
  Description of Artificial Sequence Synthetic Mutated human IGF-1 Protein
GPTLCGAELVDALQFVCGDRGFYFNKPTGYGSSSREAPQTGIVDECCFRSCDLRRLEMYCAPLKPAKSAVQAQQHTDMPKTQKEVHLKNASRGSAGNKNYRM

SEQ ID NO: 64 - protein, 102 aa, Artificial Sequence
  Description of Artificial Sequence Synthetic Mutated human IGF-1 Protein
GPTLCGAELVDALQFVCGDRGFYFNKPTGYGSSSREAPQTGIVDECCFRSCDLRRLEMYCAPLKPAKSAVQAQQHTDMPKTQKEVHLKNASRGSAGNKNYQM

SEQ ID NO: 65 - protein, 102 aa, Artificial Sequence
  Description of Artificial Sequence Synthetic Mutated human IGF-1 Protein
GPTLCGAELVDALQFVCGDRGFYFNKPTGYGSSSRAAPQTGIVDECCFRSCDLRRLEMYCAPLKPAKSAVQAQRHTDMPKTQKEVHLKNASRGSAGNKNYRM

SEQ ID NO: 66 - protein, 102 aa, Artificial Sequence
  Description of Artificial Sequence Synthetic Mutated human IGF-1 Protein
GPTLCGAELVDALQFVCGDRGFYFNKPTGYGSSSRAAPQTGIVDECCFRSCDLRRLEMYCAPLKPAKSAVQAQQHTDMPKTQKEVHLKNASRGSAGNKNYRM

SEQ ID NO: 67 - protein, 102 aa, Artificial Sequence
  Description of Artificial Sequence Synthetic Mutated human IGF-1 Protein
GPTLCGAELVDALQFVCGDRGFYFNKPTGYGSSSRAAPQTGIVDECCFRSCDLRRLEMYCAPLKPAKSAVQAQQHTDMPKTQKEVHLKNASRGSAGNKNYQM

SEQ ID NO: 68 - protein, 102 aa, Artificial Sequence
  Description of Artificial Sequence Synthetic Mutated human IGF-1 Protein
GPTLCGAELVDALQFVCGDRGFYFNKPTGYGSSSRPAPQTGIVDECCFRSCDLRRLEMYCAPLKPAKSAVQAQRHTDMPKTQKEVHLKNASRGSAGNKNYRM

SEQ ID NO: 69 - protein, 102 aa, Artificial Sequence
  Description of Artificial Sequence Synthetic Mutated human IGF-1 Protein
GPTLCGAELVDALQFVCGDRGFYFNKPTGYGSSSRPAPQTGIVDECCFRSCDLRRLEMYCAPLKPAKSAVQAQQHTDMPKTQKEVHLKNASRGSAGNKNYRM

SEQ ID NO: 70 - protein, 102 aa, Artificial Sequence
  Description of Artificial Sequence Synthetic Mutated human IGF-1 Protein
GPTLCGAELVDALQFVCGDRGFYFNKPTGYGSSSRPAPQTGIVDECCFRSCDLRRLEMYCAPLKPAKSAVQAQQHTDMPKTQKEVHLKNASRGSAGNKNYQM

SEQ ID NO: 71 - protein, 102 aa, Artificial Sequence
  Description of Artificial Sequence Synthetic Mutated human IGF-1 Protein
GPTLCGAELVDALQFVCGDRGFYFNKPTGYGSSSQQAPQTGIVDECCFRSCDLRRLEMYCAPLKPAKSAVQAQRHTDMPKTQKEVHLKNASRGSAGNKNYRM

SEQ ID NO: 72 - protein, 102 aa, Artificial Sequence
  Description of Artificial Sequence Synthetic Mutated human IGF-1 Protein
GPTLCGAELVDALQFVCGDRGFYFNKPTGYGSSSQQAPQTGIVDECCFRSCDLRRLEMYCAPLKPAKSAVQAQQHTDMPKTQKEVHLKNASRGSAGNKNYRM

SEQ ID NO: 73 - protein, 102 aa, Artificial Sequence
  Description of Artificial Sequence Synthetic Mutated human IGF-1 Protein
GPTLCGAELVDALQFVCGDRGFYFNKPTGYGSSSQAAPQTGIVDECCFRSCDLRRLEMYCAPLKPAKSAVQAQQHTDMPKTQKEVHLKNASRGSAGNKNYRM

SEQ ID NO: 74 - protein, 102 aa, Artificial Sequence
  Description of Artificial Sequence Synthetic Mutated human IGF-1 Protein
GPTLCGAELVDALQFVCGDRGFYFNKPTGYGSSSQQAPQTGIVDECCFRSCDLRRLEMYCAPLKPAKSAVQAQQHTDMPKTQKEVHLKNASRGSAGNKNYQM

SEQ ID NO: 75 - protein, 102 aa, Artificial Sequence
  Description of Artificial Sequence Synthetic Mutated human IGF-1 Protein
GPTLCGAELVDALQFVCGDRGFYFNKPTGYGSSSQAAPQTGIVDECCFRSCDLRRLEMYCAPLKPAKSAVQAQQHTDMPKTQKEVHLKNASRGSAGNKNYQM

SEQ ID NO: 76 - protein, 99 aa, Artificial Sequence
  Description of Artificial Sequence Synthetic Mutated human IGF-1 Protein
GPTLCGAELVDALQFVCGDRGFYFNKPTGYGSSSQRAPQTGIVDECCFRSCDLRRLEMYCAPLKPAVRAQRHTDMPKTQKEVHLKNASRGSAGNKNYRM

SEQ ID NO: 77 - protein, 99 aa, Artificial Sequence
  Description of Artificial Sequence Synthetic Mutated human IGF-1 Protein
GPTLCGAELVDALQFVCGDRGFYFNKPTGYGSSSREAPQTGIVDECCFRSCDLRRLEMYCAPLKPAVRAQRHTDMPKTQKEVHLKNASRGSAGNKNYRM

SEQ ID NO: 78 - protein, 99 aa, Artificial Sequence
  Description of Artificial Sequence Synthetic Mutated human IGF-1 Protein
GPTLCGAELVDALQFVCGDRGFYFNKPTGYGSSSRAAPQTGIVDECCFRSCDLRRLEMYCAPLKPAVRAQRHTDMPKTQKEVHLKNASRGSAGNKNYRM

SEQ ID NO: 79 - protein, 99 aa, Artificial Sequence
  Description of Artificial Sequence Synthetic Mutated human IGF-1 Protein
GPTLCGAELVDALQFVCGDRGFYFNKPTGYGSSSRPAPQTGIVDECCFRSCDLRRLEMYCAPLKPAVRAQRHTDMPKTQKEVHLKNASRGSAGNKNYRM

SEQ ID NO: 80 - protein, 100 aa, Artificial Sequence
  Description of Artificial Sequence Synthetic Mutated human IGF-1 Protein
GPTLCGAELVDALQFVCGDRGFYFNKPTGYGSSSQQAPQTGIVDECCFRSCDLRRLEMYCAPLKPAQVRAQRHTDMPKTQKEVHLKNASRGSAGNKNYRM

SEQ ID NO: 81 - protein, 99 aa, Artificial Sequence
  Description of Artificial Sequence Synthetic Mutated human IGF-1 Protein
GPTLCGAELVDALQFVCGDRGFYFNKPTGYGSSSQAAPQTGIVDECCFRSCDLRRLEMYCAPLKPAVRAQRHTDMPKTQKEVHLKNASRGSAGNKNYRM

SEQ ID NO: 82 - protein, 99 aa, Artificial Sequence
  Description of Artificial Sequence Synthetic Mutated human IGF-1 Protein
GPTLCGAELVDALQFVCGDRGFYFNKPTGYGSSSQRAPQTGIVDECCFRSCDLRRLEMYCAPLKPAVQAQRHTDMPKTQKEVHLKNASRGSAGNKNYRM

SEQ ID NO: 83 - protein, 99 aa, Artificial Sequence
  Description of Artificial Sequence Synthetic Mutated human IGF-1 Protein hIGF1-Ea-D1-3, R36Q, G42E, D68-72, R74Q, R77Q, R104Q -fc domain
GPTLCGAELVDALQFVCGDRGFYFNKPTGYGSSSQRAPQTGIVDECCFRSCDLRRLEMYCAPLKPAVQAQQHTDMPKTQKEVHLKNASRGSAGNKNYRM

SEQ ID NO: 84 - protein, 99 aa, Artificial Sequence
  Description of Artificial Sequence Synthetic Mutated human IGF-1 Protein
GPTLCGAELVDALQFVCGDRGFYFNKPTGYGSSSQRAPQTGIVDECCFRSCDLRRLEMYCAPLKPAVQAQQHTDMPKTQKEVHLKNASRGSAGNKNYQM

SEQ ID NO: 85 - protein, 99 aa, Artificial Sequence
  Description of Artificial Sequence Synthetic Mutated human IGF-1 Protein
GPTLCGAELVDALQFVCGDRGFYFNKPTGYGSSSREAPQTGIVDECCFRSCDLRRLEMYCAPLKPAVQAQRHTDMPKTQKEVHLKNASRGSAGNKNYRM

SEQ ID NO: 86 - protein, 99 aa, Artificial Sequence
  Description of Artificial Sequence Synthetic Mutated human IGF-1 Protein
GPTLCGAELVDALQFVCGDRGFYFNKPTGYGSSSREAPQTGIVDECCFRSCDLRRLEMYCAPLKPAVQAQQHTDMPKTQKEVHLKNASRGSAGNKNYRM

SEQ ID NO: 87 - protein, 99 aa, Artificial Sequence
  Description of Artificial Sequence Synthetic Mutated human IGF-1 Protein
GPTLCGAELVDALQFVCGDRGFYFNKPTGYGSSSREAPQTGIVDECCFRSCDLRRLEMYCAPLKPAVQAQQHTDMPKTQKEVHLKNASRGSAGNKNYQM

SEQ ID NO: 88 - protein, 99 aa, Artificial Sequence
  Description of Artificial Sequence Synthetic Mutated human IGF-1 Protein
GPTLCGAELVDALQFVCGDRGFYFNKPTGYGSSSRAAPQTGIVDECCFRSCDLRRLEMYCAPLKPAVQAQRHTDMPKTQKEVHLKNASRGSAGNKNYRM

SEQ ID NO: 89 - protein, 99 aa, Artificial Sequence
  Description of Artificial Sequence Synthetic Mutated human IGF-1 Protein
GPTLCGAELVDALQFVCGDRGFYFNKPTGYGSSSRAAPQTGIVDECCFRSCDLRRLEMYCAPLKPAVQAQQHTDMPKTQKEVHLKNASRGSAGNKNYRM

SEQ ID NO: 90 - protein, 99 aa, Artificial Sequence
  Description of Artificial Sequence Synthetic Mutated human IGF-1 Protein
GPTLCGAELVDALQFVCGDRGFYFNKPTGYGSSSRAAPQTGIVDECCFRSCDLRRLEMYCAPLKPAVQAQQHTDMPKTQKEVHLKNASRGSAGNKNYQM

SEQ ID NO: 91 - protein, 99 aa, Artificial Sequence
  Description of Artificial Sequence Synthetic Mutated human IGF-1 Protein
GPTLCGAELVDALQFVCGDRGFYFNKPTGYGSSSRPAPQTGIVDECCFRSCDLRRLEMYCAPLKPAVQAQRHTDMPKTQKEVHLKNASRGSAGNKNYRM

SEQ ID NO: 92 - protein, 99 aa, Artificial Sequence
  Description of Artificial Sequence Synthetic Mutated human IGF-1 Protein
GPTLCGAELVDALQFVCGDRGFYFNKPTGYGSSSRPAPQTGIVDECCFRSCDLRRLEMYCAPLKPAVQAQQHTDMPKTQKEVHLKNASRGSAGNKNYRM

SEQ ID NO: 93 - protein, 99 aa, Artificial Sequence
  Description of Artificial Sequence Synthetic Mutated human IGF-1 Protein
GPTLCGAELVDALQFVCGDRGFYFNKPTGYGSSSRPAPQTGIVDECCFRSCDLRRLEMYCAPLKPAVQAQQHTDMPKTQKEVHLKNASRGSAGNKNYQM

SEQ ID NO: 94 - protein, 99 aa, Artificial Sequence
  Description of Artificial Sequence Synthetic Mutated human IGF-1 Protein
GPTLCGAELVDALQFVCGDRGFYFNKPTGYGSSSQQAPQTGIVDECCFRSCDLRRLEMYCAPLKPAVQAQRHTDMPKTQKEVHLKNASRGSAGNKNYRM

SEQ ID NO: 95 - protein, 99 aa, Artificial Sequence
  Description of Artificial Sequence Synthetic Mutated human IGF-1 Protein
GPTLCGAELVDALQFVCGDRGFYFNKPTGYGSSSQQAPQTGIVDECCFRSCDLRRLEMYCAPLKPAVQAQQHTDMPKTQKEVHLKNASRGSAGNKNYRM

SEQ ID NO: 96 - protein, 99 aa, Artificial Sequence
  Description of Artificial Sequence Synthetic Mutated human IGF-1 Protein
GPTLCGAELVDALQFVCGDRGFYFNKPTGYGSSSQAAPQTGIVDECCFRSCDLRRLEMYCAPLKPAVQAQQHTDMPKTQKEVHLKNASRGSAGNKNYRM

SEQ ID NO: 97 - protein, 99 aa, Artificial Sequence
  Description of Artificial Sequence Synthetic Mutated human IGF-1 Protein
GPTLCGAELVDALQFVCGDRGFYFNKPTGYGSSSQQAPQTGIVDECCFRSCDLRRLEMYCAPLKPAVQAQQHTDMPKTQKEVHLKNASRGSAGNKNYQM

SEQ ID NO: 98 - protein, 99 aa, Artificial Sequence
  Description of Artificial Sequence Synthetic Mutated human IGF-1 Protein
GPTLCGAELVDALQFVCGDRGFYFNKPTGYGSSSQAAPQTGIVDECCFRSCDLRRLEMYCAPLKPAVQAQQHTDMPKTQKEVHLKNASRGSAGNKNYQM

SEQ ID NO: 99 - protein, 23 aa, Artificial Sequence
  Description of Artificial Sequence Synthetic mutated Human IGF1 E-peptide
VQAQQHTDMPKTQKEVHLKNASG

SEQ ID NO: 100 - protein, 32 aa, Artificial Sequence
  Description of Artificial Sequence Synthetic mutated Human IGF1 E-peptide
VQAQQHTDMPKTQKYQPPATNKNTKSQRRKGS

SEQ ID NO: 101 - protein, 33 aa, Artificial Sequence
  Description of Artificial Sequence Synthetic mutated Human IGF1 E-peptide
VQAQQHTDMPKTQKEVHLKNASRGSAGNKNYQM